SEQUENCE LISTING (11 sequences)

SEQ ID NO: 1 - protein, 15 aa, Artificial sequence
  Synthetic peptide
VVSEDFLQDVSASTK

SEQ ID NO: 2 - protein, 14 aa, Artificial sequence
  Synthetic peptide
HQSFVLVGETGSGK

SEQ ID NO: 3 - DNA, 20 bp, Artificial sequence
  Synthetic primer
gagtcaacggatttggtcgt

SEQ ID NO: 4 - DNA, 20 bp, Artificial sequence
  Synthetic primer
gacaagcttcccgttctcag

SEQ ID NO: 5 - DNA, 21 bp, Artificial sequence
  Synthetic primer
tggaaaagtcccacactggta

SEQ ID NO: 6 - DNA, 21 bp, Artificial sequence
  Synthetic primer
aagctcagagaacccatccac

SEQ ID NO: 7 - DNA, 21 bp, Artificial sequence
  Synthetic primer
ggcacaaatcaaggcaggtta

SEQ ID NO: 8 - DNA, 21 bp, Artificial sequence
  Synthetic primer
aagtcatgcggaatcctggtg

SEQ ID NO: 9 - DNA, 23 bp, Artificial sequence
  Synthetic primer
gaccaacatcgagaacaacaaca

SEQ ID NO: 10 - DNA, 21 bp, Artificial sequence
  Synthetic primer
gccttgtgaagtggttgatct

SEQ ID NO: 11 - protein, 10 aa, Artificial sequence
  Synthetic peptide
AEPVEVVAPR

(19) United States
(12) Patent Application Publication
(10) Pub. No.: US 2022/0098196 A1
(43) Pub. Date: Mar. 31, 2022

(54) TRAPPING-FREE PARP INHIBITORS

(71) Applicant: The Board of Regents of the University of Texas System, Austin, TX (US)

(72) Inventors: Yonghao YU, Dallas, TX (US); Chuo CHEN, Dallas, TX (US); Shuai WANG, Dallas, TX (US); Lei HAN, Dallas, TX (US); Lin YOU, Dallas, TX (US)

(73) Assignee: The Board of Regents of the University of Texas System, Austin, TX (US)

(21) Appl. No.: 17/428,605

(22) PCT Filed: Jan. 31, 2020
(86) PCT No.: PCT/US2020/016129

§ 371 Date: Aug. 4, 2021

Related U.S. Application Data
(60) Provisional application No. 62/801,453, filed on Feb. 5, 2019.

(51) Int. Cl. C07D 471/06; C07D 401/14; C07D 417/14; C07D 487/06; A61K 45/06

(52) U.S. Cl. CPC C07D 471/06; C07D 401/14; A61K 47/55; C07D 487/06; A61K 45/06; C07D 417/14

(57) ABSTRACT

The disclosure provides PARP PROTAC molecules comprising a PARP inhibitor linked to an E3 ligase binder, and their use in treating diseases caused by aberrant PARP activation. Such PROTAC molecules produce selective degradation of one or a specific set of PARP proteins with limited effects on the protein level of other PARPs.

### PRIORITY CLAIM

This application claims benefit of priority to U.S. Provisional Application Ser. No. 62/801,453, filed Feb. 5, 2019, the entire contents of which are hereby incorporated by reference.

### GOVERNMENT SUPPORT

This invention was made with government support under contract number GM122932 awarded by the National Institutes of Health. The government has certain rights in the invention.

### BACKGROUND

### I. Field

The field of the disclosure relates generally to molecular biology, pharmacology, medicine, oncology, and chemotherapy. More specifically, the disclosure relates to PARP1-targeting PROTACs comprising a PARP1 inhibitor and a binding agent directed to either CRBN of VHL.

### II. Related Art

PARP1 is an abundant nuclear protein that is critically involved in a number of biological processes linked to cellular stress responses (D'Amours et al. 1999; Gibson & Kraus, 2012; Hottiger, 2015). The enzymatic function of PARP1 is to catalyze a protein posttranslational modification called Poly-ADP-ribosylation (PARylation) (Chambon et al. 1963). PARylation is composed of linear and/or branched repeats of ADP-ribose, with lengths reaching up to 200 units (D'Amours et al., 1999). Compared to other common PTMs, a rather unique feature about ADP-ribosylation is that this modification can occur on amino acids with diverse side chain chemistries, including Asp, Glu, Lys, Arg, Cys, Ser and Tyr (Daniels et al., 2014; Leidecker et al., 2016; Pedrioli et al., 2018; Leutert et al., 2018; Martello et al., 2016; Zhang et al., 2013). Besides PARP1, numerous efforts have now identified 16 additional PARP enzymes (Wahlberg et al., 2012).

PARP1 and PARP1-medaited PARylation events are tightly connected to DNA damage response (DDR) (Liu et al., 2017). The PARylation level in a quiescent cell is usually very low. In response to genotoxic stress, PARP1 binds to nicked DNA and is rapidly activated. This leads to the generation of a large number of PARylated proteins and subsequently, the initiation of the DNA damage repair program (Krishnakumar & Kraus, 2010). From a structural perspective, PAR resembles DNA, both of which are bulky, charged and flexible. The interaction between DNA and a DNA-binding protein could therefore be disrupted as a result of its covalent modification by PAR (Miyamoto et al., 1999). In addition, PAR polymers may also act as a scaffold for recruiting other proteins that bear PAR-binding motifs (PBMs), including OB, WWE, PBZ, BRCT and macrodomain. These PBMs bind to different structural units within PAR (e.g., ADP-ribose and iso-ADP-ribose), and they are present in many proteins involved in DDR (Liu et al., 2017).

The critical role of PARP1 in mediating DDR provides the rationale for developing PARP1 inhibitors to treat human cancer (Lord & Ashworth, 2017). In particular, BRCA1/2 are tumor suppressor proteins that play a critical role in mediating DNA double-strand break (DSB) repair. They are mutated in about 10% breast cancers and 15% ovarian cancers (Campeau et al., 2008; Pal et al., 2005). It has been shown that BRCA1/2-mutated cancer cells are deficient for homologous recombination. As a result, they rely on PARP1 for genome integrity, and can be selectively targeted by PARP1 inhibitors through the concept of “synthetic lethality” (Bryant et al., 2005; Farmer et al., 2005). Besides blocking the formation of PAR polymers, a number of recent studies suggest that PARPi (PARP1 inhibitors) may also kill tumor cells via a “trapping” mechanism (Murai et al., 2012). Specifically, upon sensing nicked DNA, PARP1 is activated and automodified. Auto-PARylation of PARP1 results in its dissociation from DNA, due to steric hindrance and charge repulsion. Upon the treatment of PARPi, PARP1 is unable to modify itself, and thus is trapped at the DNA lesion. It has been suggested that this PARP1-PARPi-DNA complex interferes with subsequent DNA replication and therefore is highly cytotoxic (Lord & Ashworth, 2017). Although this model suggests that catalytic inhibition of PARP1 is functionally inequivalent to PARP1 deletion, there lacks a pharmacological approach to dissecting these two pathways (i.e., PARP1 catalytic inhibition vs PARP1 trapping), in order to assess their relative contribution to the cytotoxicity of PARPi.

Besides the role as an oncology target, PARP1 is hyperactivated upon sensing genotoxic stresses associated with various pathological conditions (e.g., ischemia-reperfusion (IR) injury (Berger et al., 2018) and neurodegeneration (Brochier et al., 2015). Over-stimulation of PARP1 could account for more than 90% of the PAR synthesized under these conditions, representing the primary mechanism for NAD⁺ catabolism (Shieh et al., (1998). Depletion of the cellular NAD⁺ pool leads to forced NAD⁺ synthesis through the salvage pathway. This, in turn, lowers the cellular ATP level and eventually, causes cell death (i.e., necrosis) (D'Amours et al., 1999). Recent studies have shown that the accumulation of PAR could also lead to the translocation of apoptosis-inducing factor (AIF) from mitochondria to the nucleus, and cause another form of cell death known as parthanatos (Yu et al., 2002; Wang et al., 2009). PARP1 thus is also being evaluated as a therapeutic target for IR injury. However, because of the significant cytotoxicity associated with PARP1 trapping, PARP1 inhibition with current inhibitors may not be useful in this context. Towards this, the development of selective PARP1 inhibitors that mimic PARP1 genetic depletion (i.e., inhibit PARP1 without causing PARP1 trapping) will likely have substantial therapeutic potential.

Recently, a number of groups reported a strategy in which a small molecule ligand of a target protein is linked to a binder of a ubiquitin E3 ligase. In doing so, this bivalent agent brings the target protein and the E3 ligase into proximity where this protein is ubiquitinated and subsequently degraded through the proteasome pathway. This strategy, termed proteolysis targeting chimera (PROTAC), has been designed utilizing small molecule binders of various E3 ligases (e.g., cereblon/CRBN, VHL, MDM2, etc.) (Burslem & Crews, 2017). By conjugating these binders to a ligand for a protein of interest, it demonstrated induced protein degradation of a multitude of targets, including RIPK2, BCR-ABL, BRD4 and CDK9, etc. (Bondeson et al., 2015; Lai et al., 2016; Winter et al., 2015; Lu et al., 2015, Olson et al., 2018). PROTACs offer a range of unique advantages over catalytic inhibition, including great versatility (a plug-and-play mode of action), a prolonged effect, and the ability to inhibit the activity of “undruggable” proteins. Importantly, by pharmacological depletion of a protein of interest, PROTACs are able to abrogate the non-enzyme-dependent (e.g., scaffolding) function of this protein (Burslem & Crews, 2017).

### SUMMARY

Thus, in accordance with the present disclosure, there is provided A compound of Formula (I), or a pharmaceutically acceptable salt thereof,

A-L-B (I)

wherein:
A is or comprises a binding moiety that binds to a PARP protein;
B is or comprises a binding moiety that binds to a E3 ligase for ubiquitin or a ubiquitin-like protein; and
L is a linker moiety that connects A and B in a manner that allows A and B to establish appropriate interactions with a PARP protein and an E3 ligase, respectively.
   A may make one or more interactions with PARP1, PARP2, PARP3, PARP5A or PARP5B. B may bind to cereblon (CRBN), von Hippel Lindau disease tumor suppressor (pVHL), mouse double minute 2 homolog (MDM2), cellular inhibitor of apoptosis protein-1 (cIAP1), or beta-transducin repeat containing protein (β-TrCP).

A may make one or more interactions with human PARP1 at one or more sites selected from the group consisting of Glu763, Asp766, His862, Gly863, Arg878, Ala880, Gly888, Tyr889, Tyr896, Ser904, and Glu988. A may make one or more interactions with human PARP2 at one or more sites selected from the group consisting of Gly429, Arg444, Tyr462, Ser470, and Tyr473. A may make one or more interactions with human PARP3 at one or more sites selected from the group consisting of Leu287, Asp291, His384, Gly385, Thr386, Arg400, Tyr414, and Ser422. A may make one or more interactions with human PARP5A at one or more sites selected from the group consisting of Gly1185, Asp1198, His1201, Tyr1203, Tyr1213, and Ser1221. A may make one or more interactions with human PARP5B at one or more sites selected from the group consisting of Gly1032, Asp1045, His1048, Tyr1050, Tyr4060, and Ser1068. A may be rucaparib, veliparib, niraparib, olaparib, talazoparib, AG-14361, GPI 15427, GPI 16539, GPI 21016, CEP-3499, CEP-8983, PJ34, PD128763, NU1025, NU1085, PA-10, OL-1, STO1168, STO1131, STO1542, ME0327, ME0328, ME0352, ME0354, ME0355, ME0359, ME0368, ME0398, ME0400, isoindolin-1-one, 4-benzylphthalazin-1(2H)-one, 2,7,8,9-tetrahydro-3H-pyrido[4,3,2-de]phthalazin-3-one, 5-oxo-6,11-dihydro-5H-indeno[1,2-c]isoquinoline-9-sulfonamide, pyrazolo[1,5-a]quinazolin-5(4H)-one, or 3-oxo-2,3-dihydrobenzofuran-7-carboxamide. A may also be IWR-1, IWR-3, IWR-6, IWR-8, XAV939, GNF-1331, GNF-6231, AZ-6102, TNKSi49, AZ-1366, AZ-6102, CMP4, CMP4b, CMP24, CMP40, JW-55, JW-74, G007-LK, K-756, NVP-TNKS656, UPF-1854, E7449, K-756, or WIKI-4. B may be thalidomide, pomalidomide, or lenalidomide, or may comprise a 4-hydroxyprolyl derivative. L may comprise a linear chain with a formula of —[(CH₂)ₘ₁—X₁]ₙ₁—[(CH₂)ₘ₂—X₂]ₙ₂—[(CH₂)ₘ₃—X₃]ₙ₃—[(CH₂)ₘ₄—X₄]ₙ₄—, wherein —[(CH₂)ₘ₁—X₁]ₙ₁ is covalently bound to A, [(CH₂)ₘ₄—X₄]ₙ₄— is covalently bound to B, each m1, m2, m3, and m4 is independently 0, 1, 2, 3, 4, 5, 5, 7, 8, 9, or 10, each n1, n2, n3, and n4 is independently 0, 1, 2, 3, 4, 5, 5, 7, 8, 9, or 10, and each X1, X2, X3, and X4 is independently absent (a bond), O, S, NH, NR, C(O), C(O)O, OC(O), C(O)NH, NHC(O), C(O)NR, N(R)C(O),

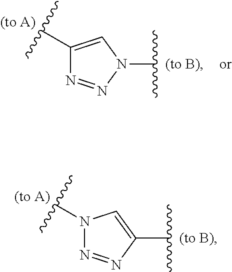

wherein R is C₁₋₆alkyl, C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups, C₃₋₅alkenyl, C₃₋₅alkynyl, oligo(ethylene glycol), or poly(ethylene glycol), wherein R is optionally conjugated to an antibody. L may alternatively comprise an oligo(ethylene glycol) chain.

The compound may be of formula (IIa), (IIb), (IIc), (IId), (IIe), (IIf), (IIg), (IIh), (IIi) or (IIj), or a pharmaceutically acceptable salt thereof:

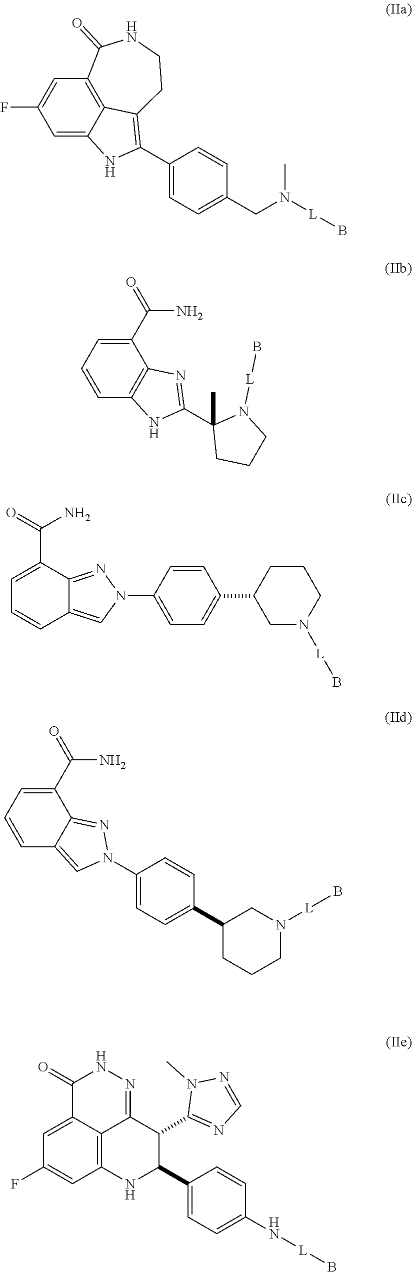

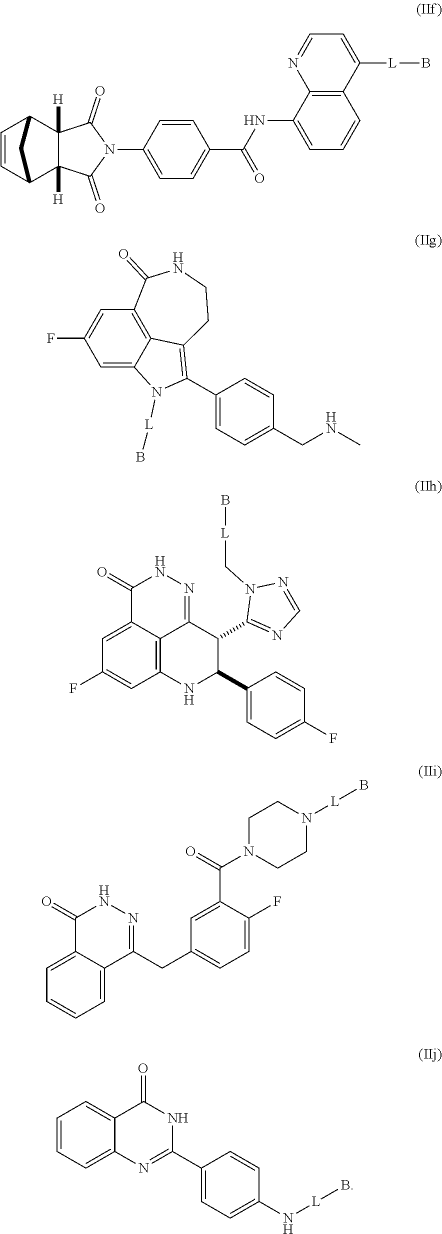

Alternatively, the compound may e of formula (IIa), (IIIb), (IIIc), (IIId), (IIIe), (IIIf), (IIIg), (IIIh), (IIIl) or (IIIj), or a pharmaceutically acceptable salt thereof:

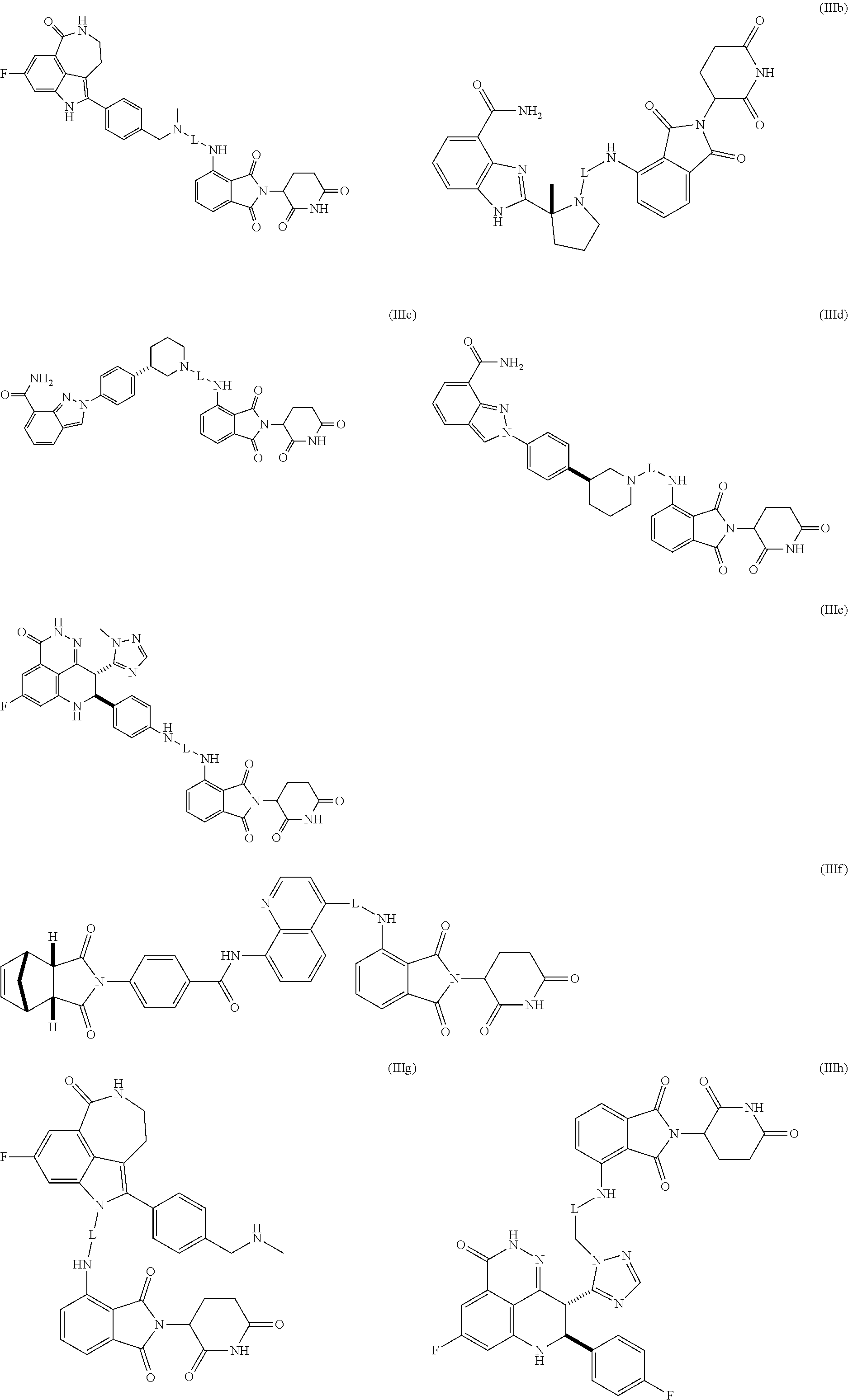

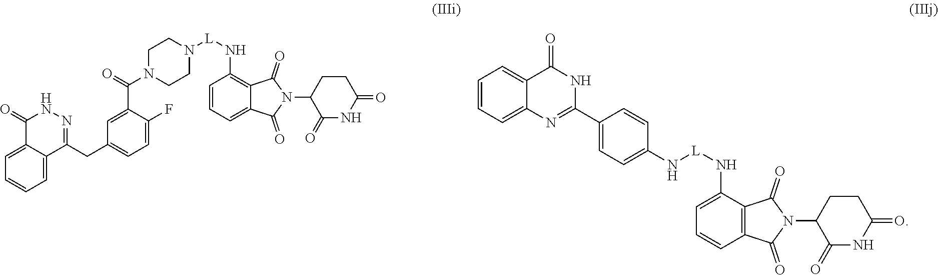

Alternatively, the compound may be of formula (IVa), (IVb), (IVc), (IVd), (IVe), (IVf), (IVg), (IVh), (IVi) or (IVj), or a pharmaceutically acceptable salt thereof:

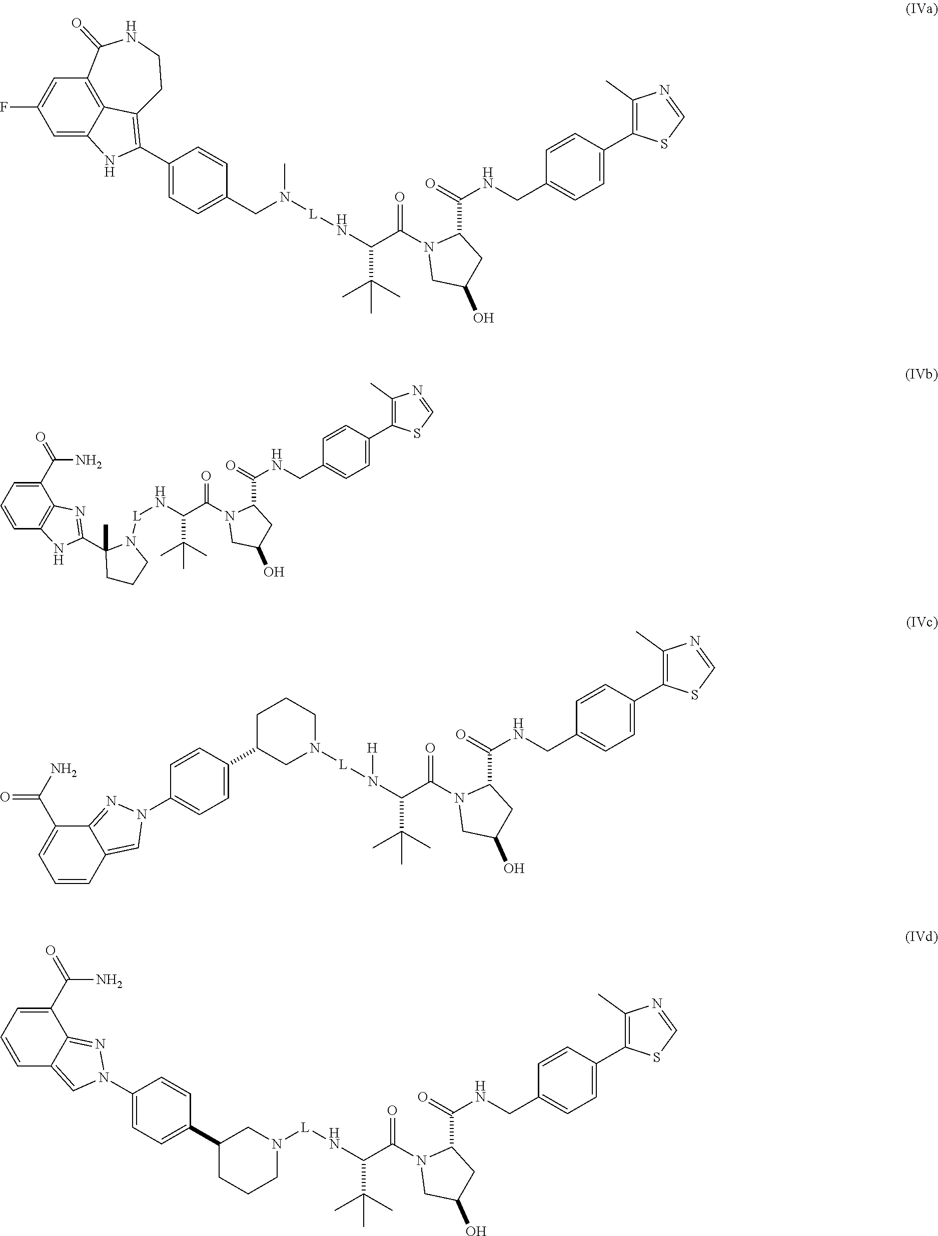

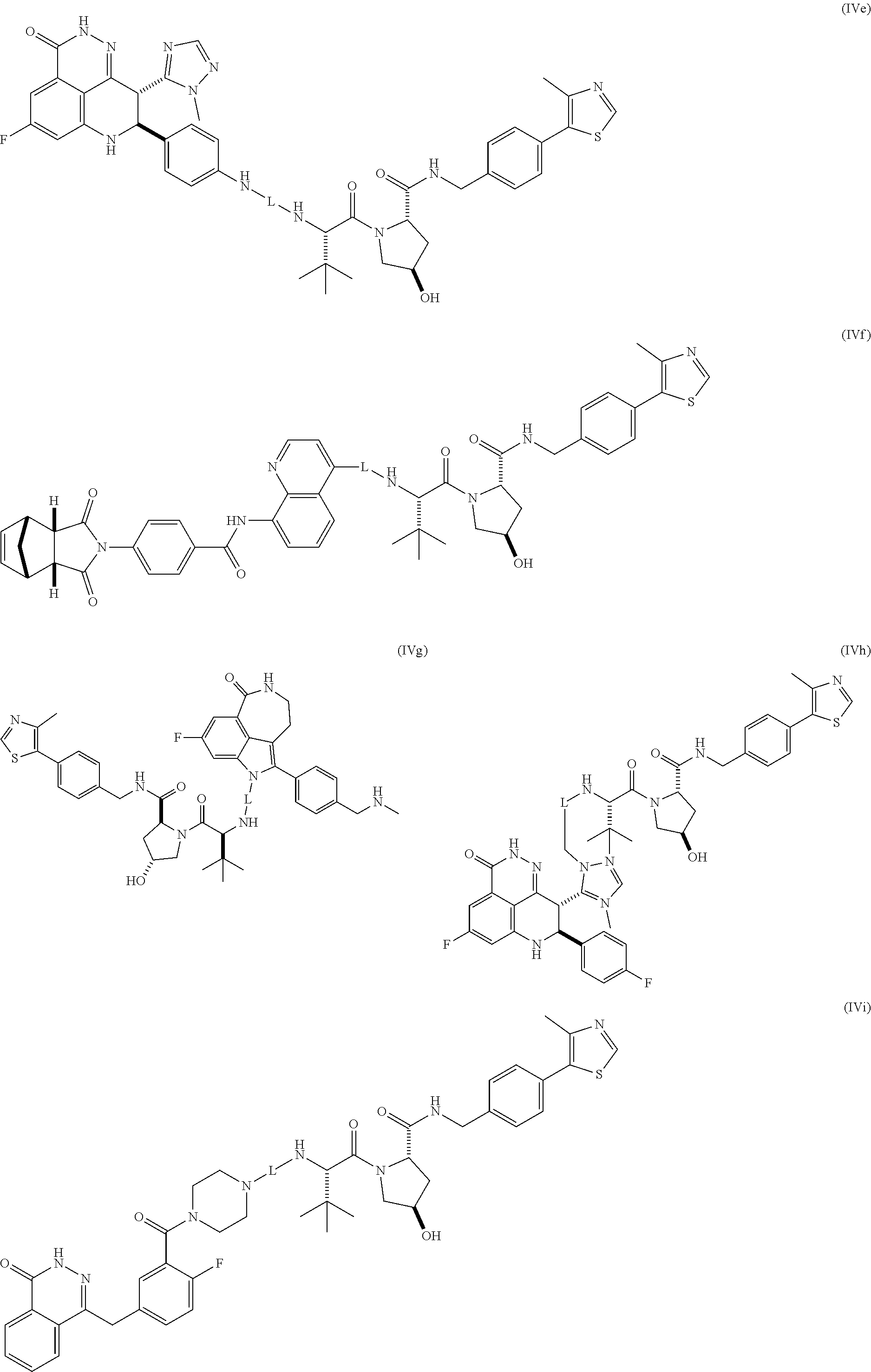

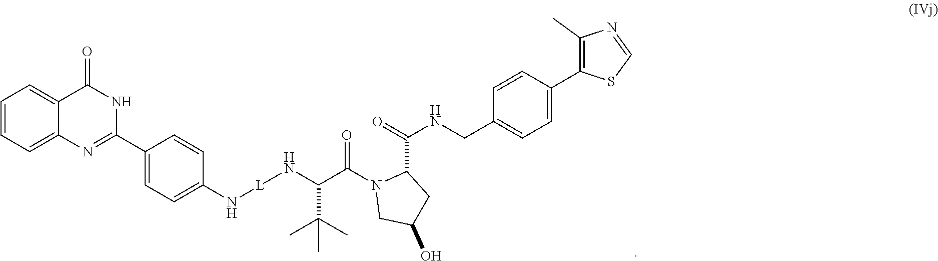

Alternatively, the compound may be of formula (Va), (Vb), (Vc), (Vd), (Ve), (Vf), (Vg), (Vh), (Vi), (Vj), (Vk), (Vl), (Vm) or (Vn), or a pharmaceutically acceptable salt thereof, wherein n is 0, 1, 2, 3, 4, 5, 5, 7, 8, 9, or 10:

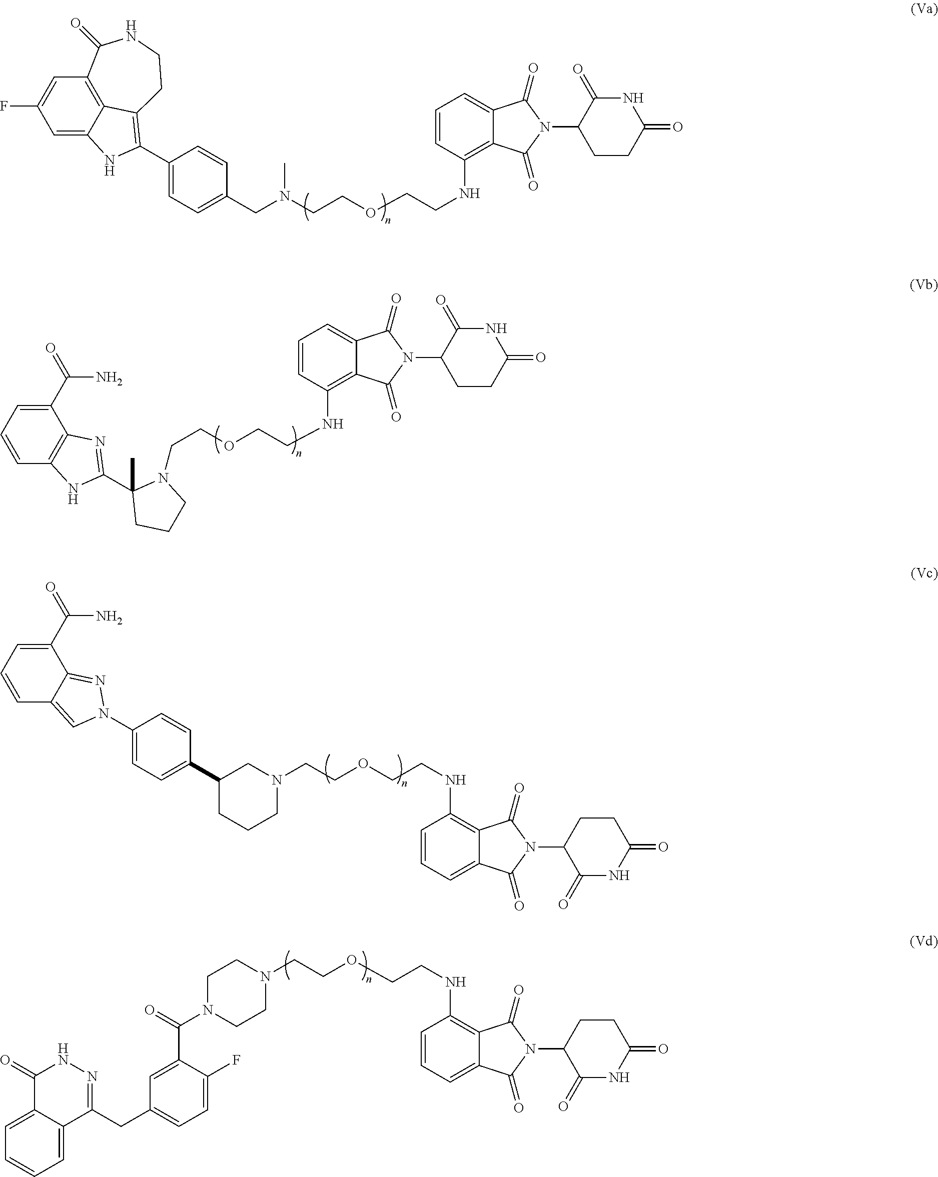

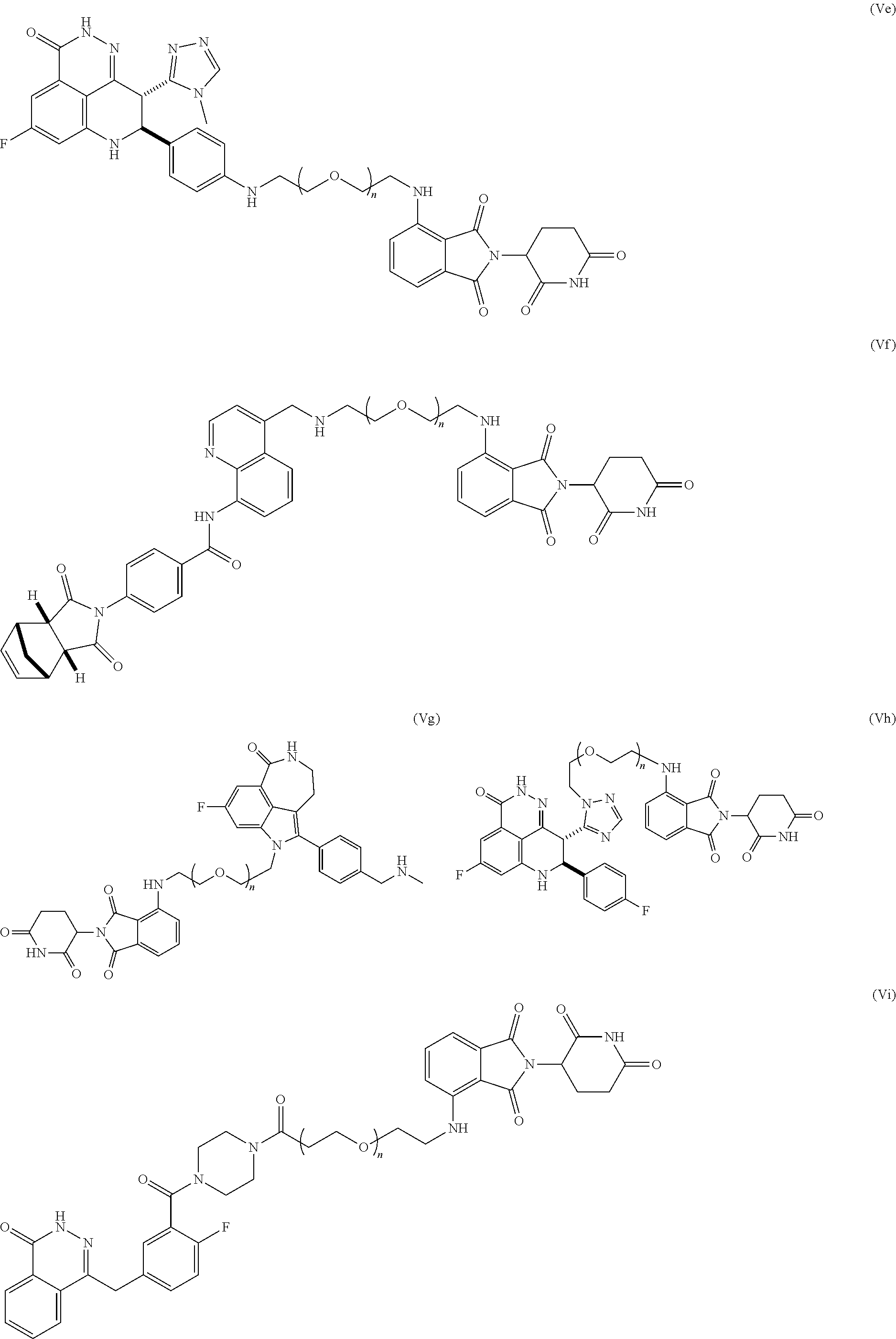

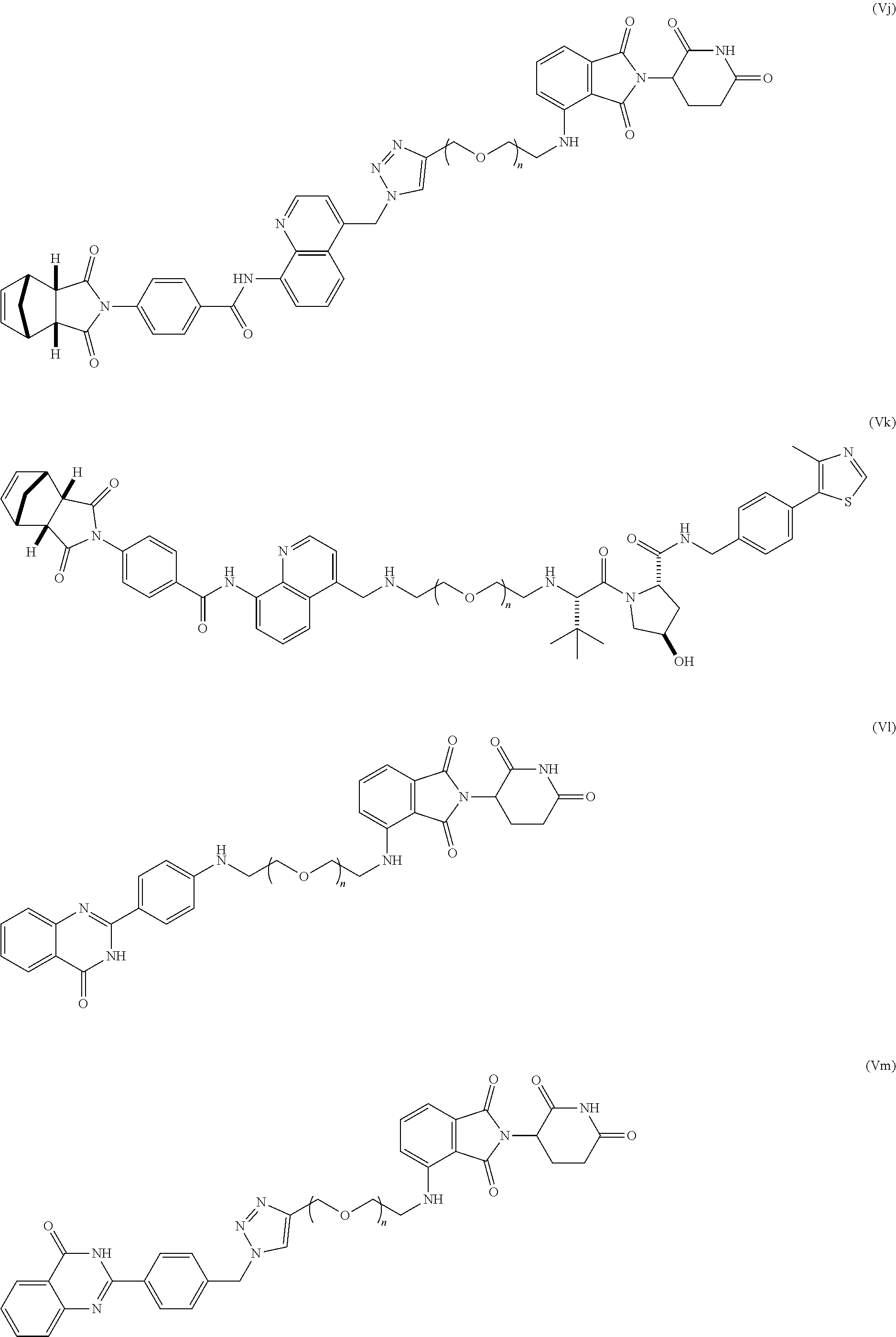

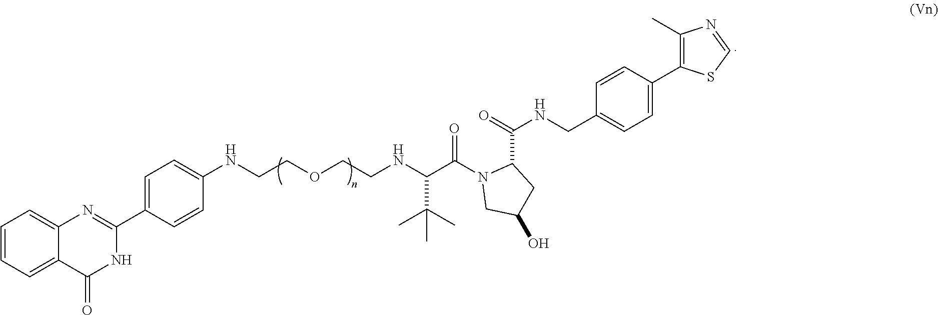

The compound may be selected from the group consisting of:

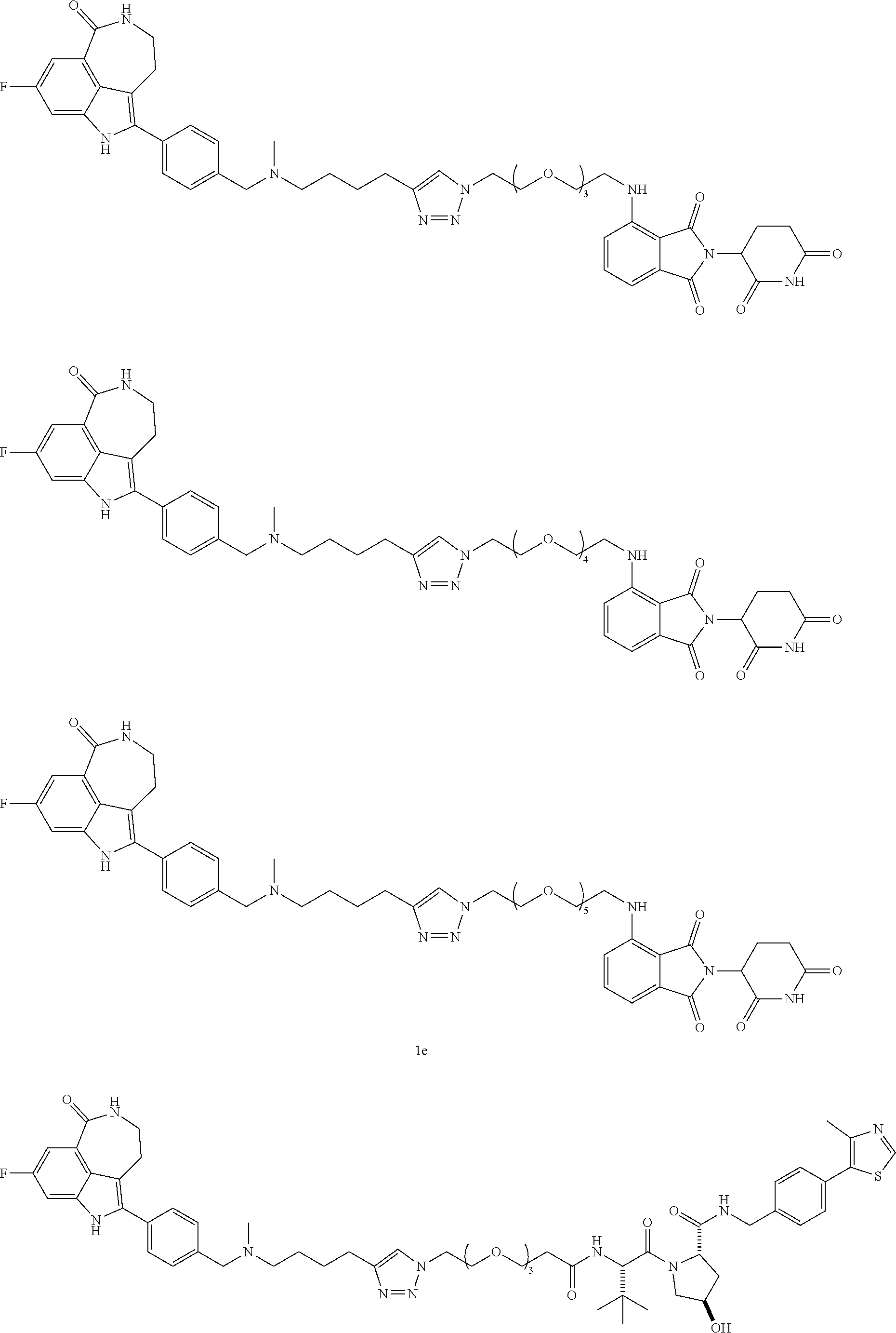

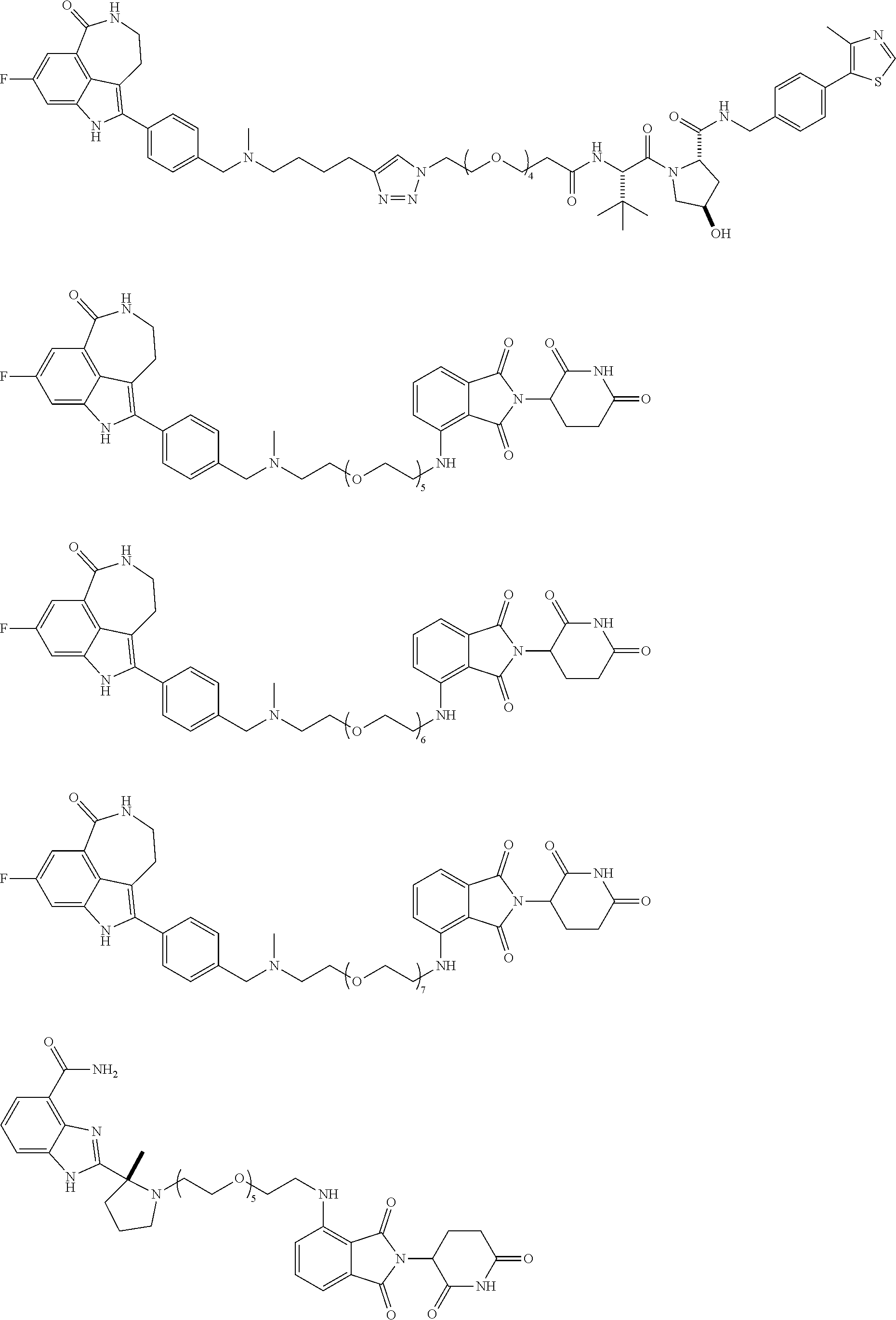

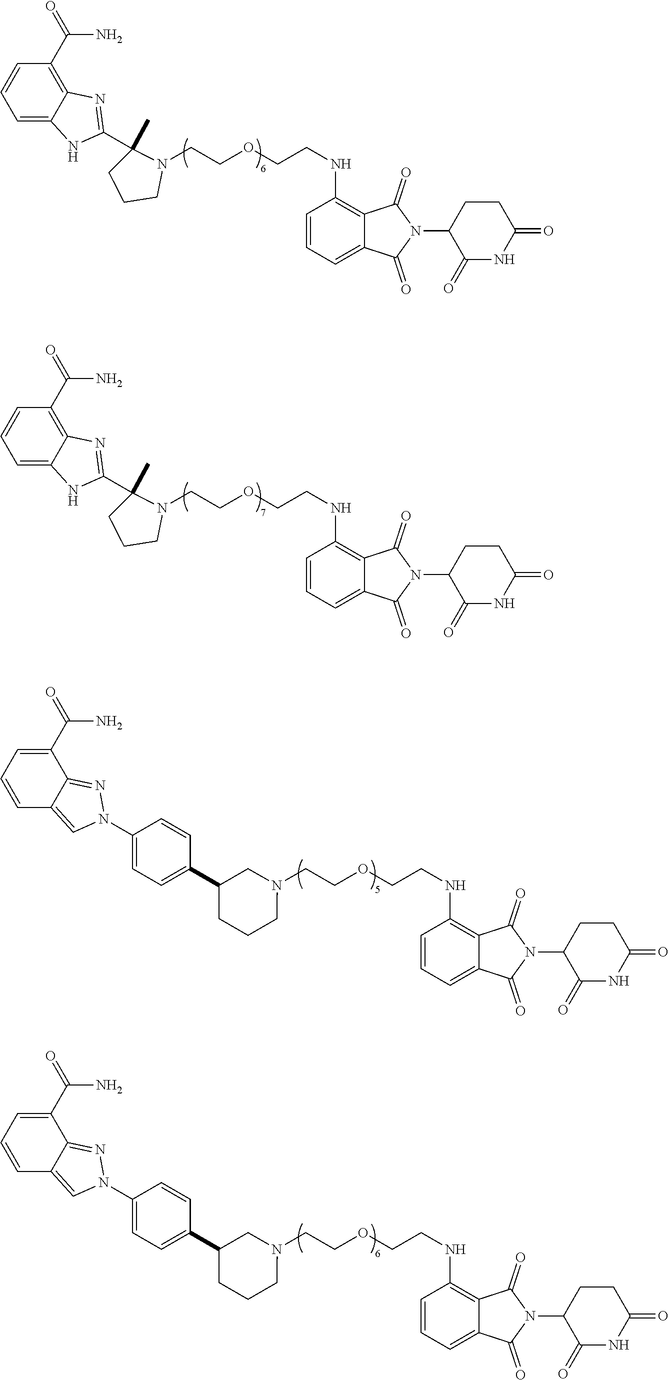

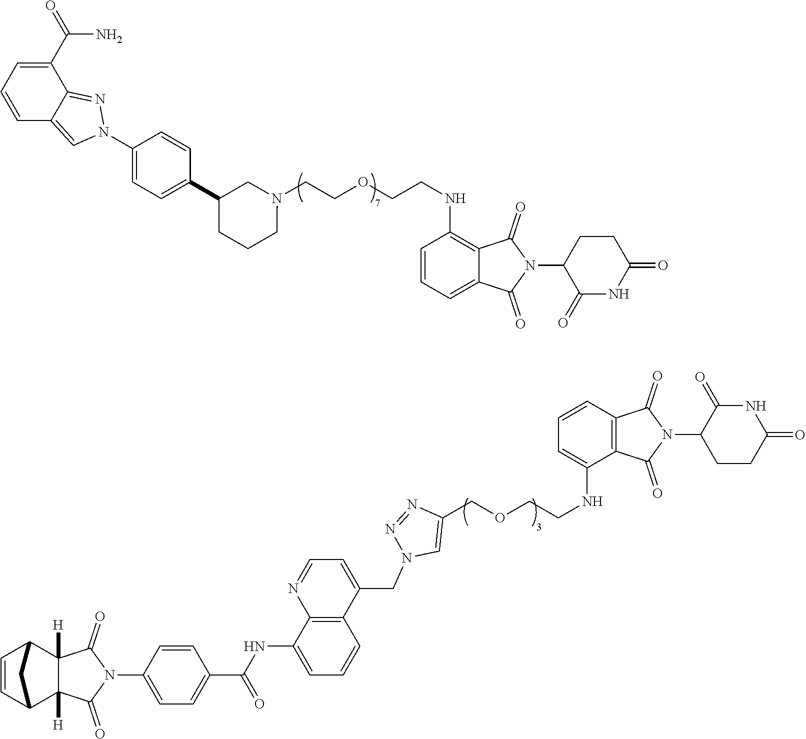

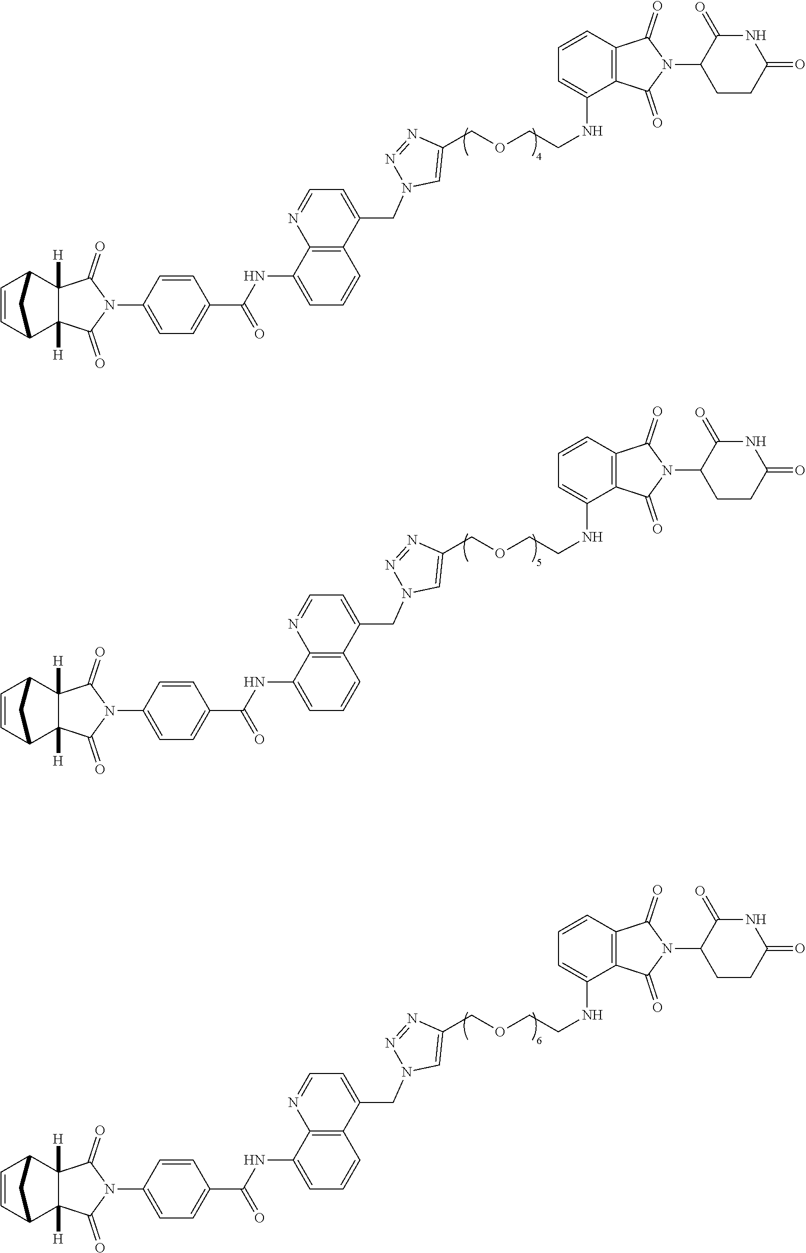

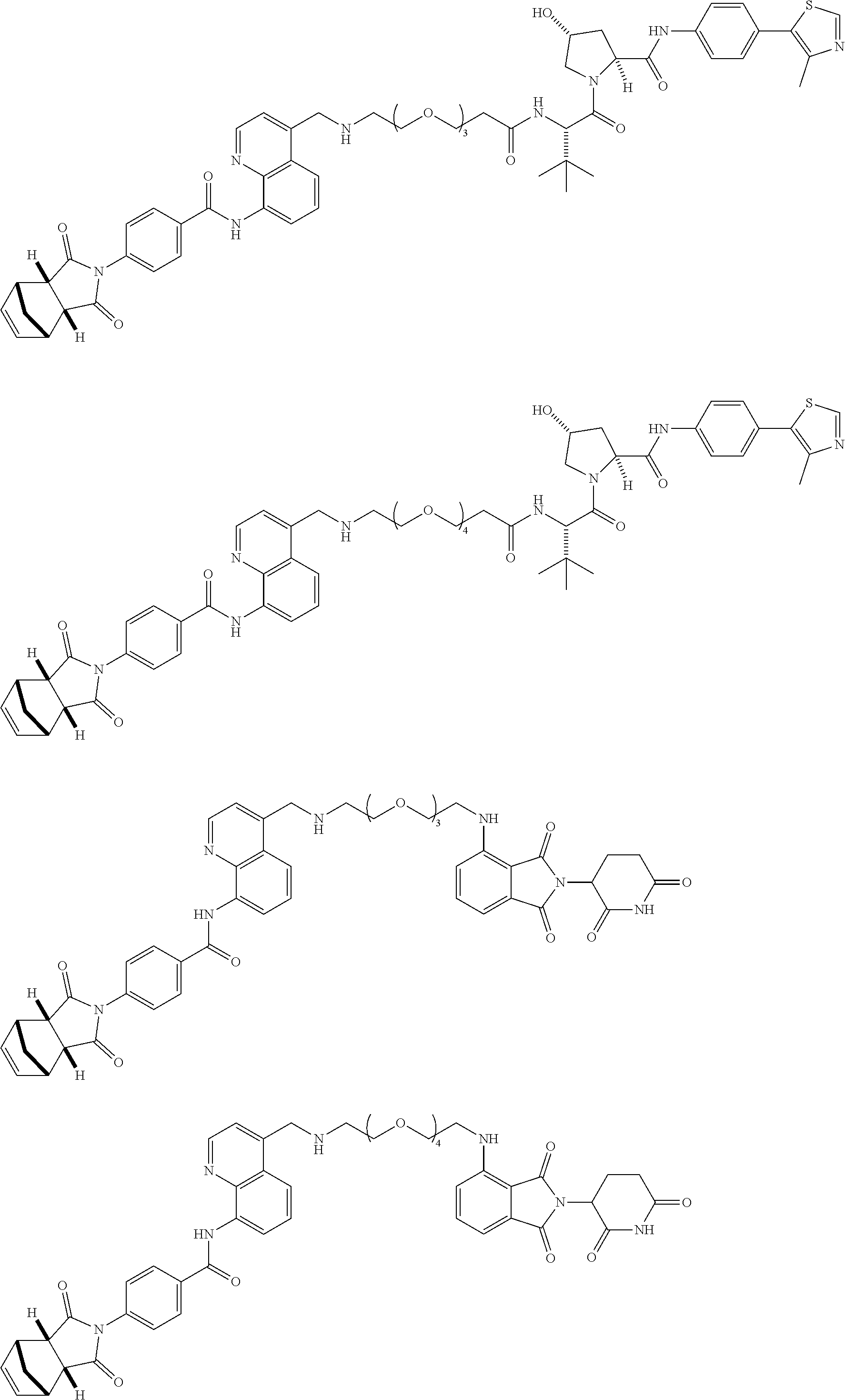

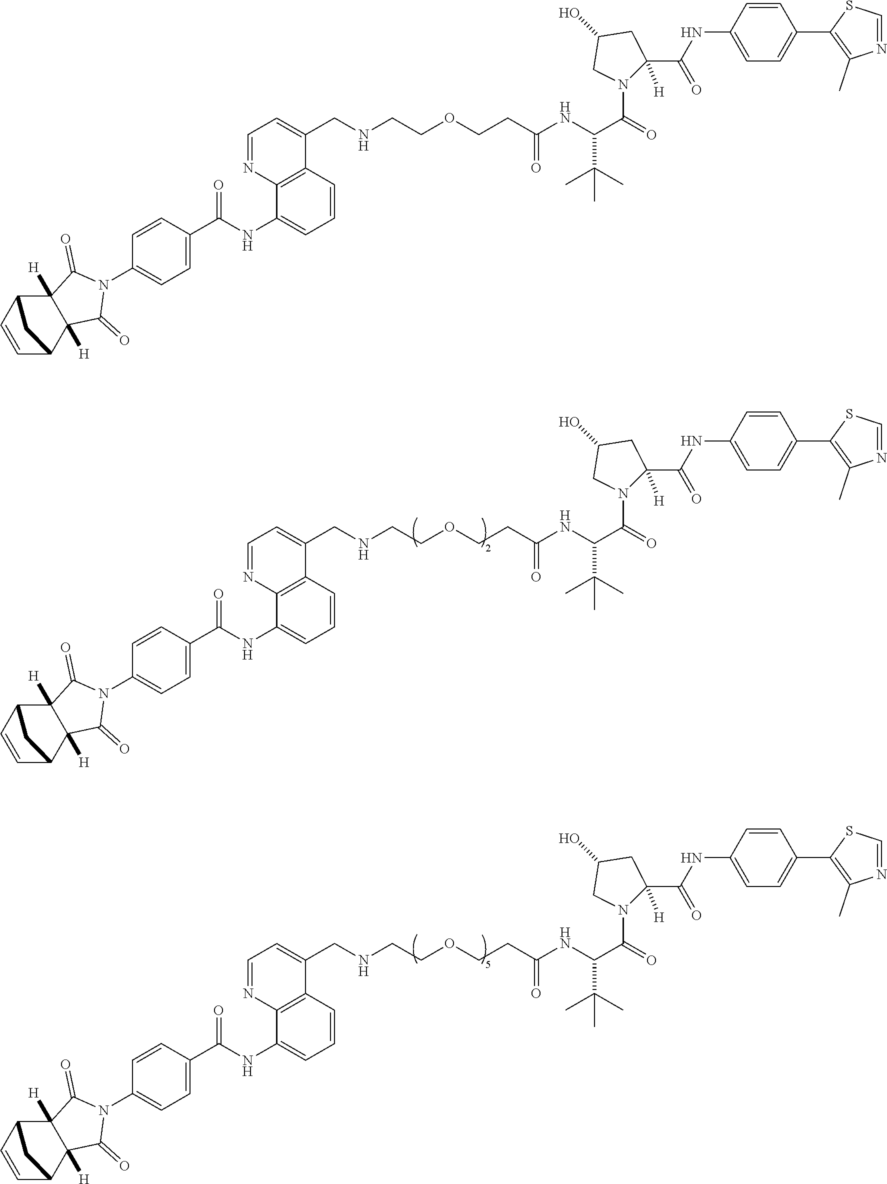

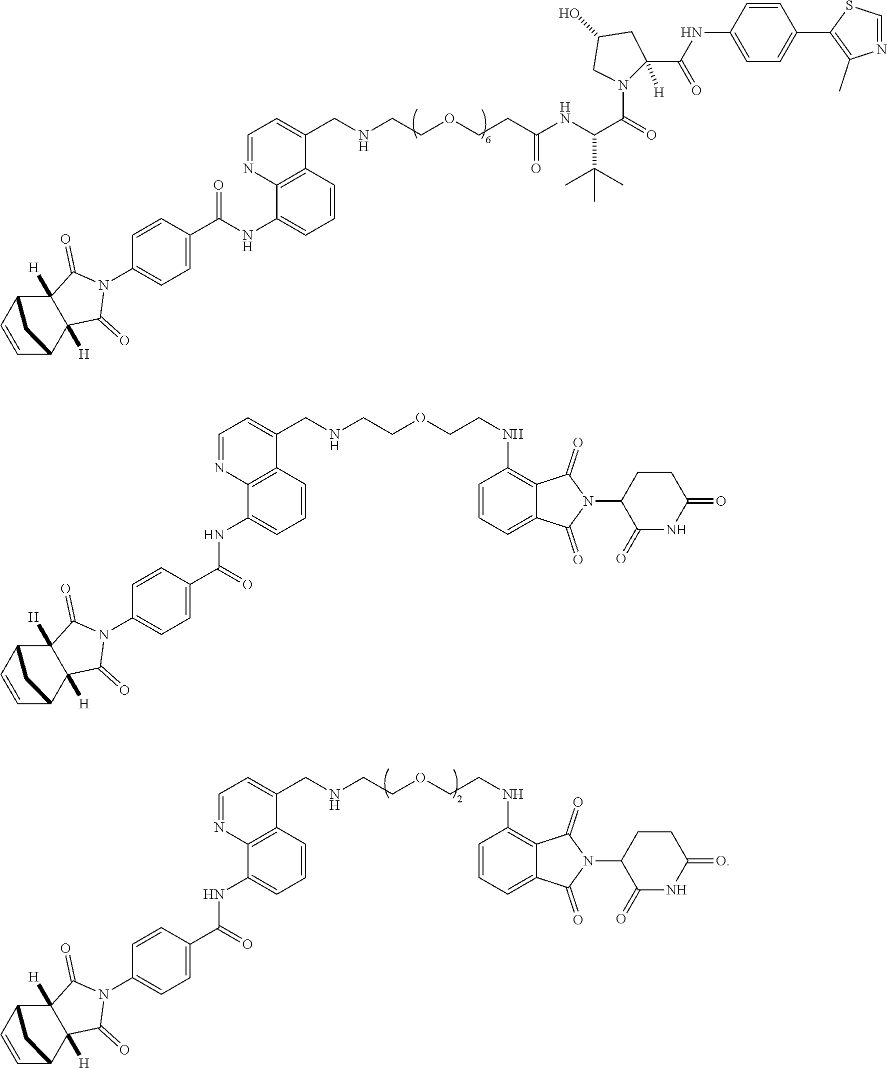

The compound may be selected from the group consisting of:

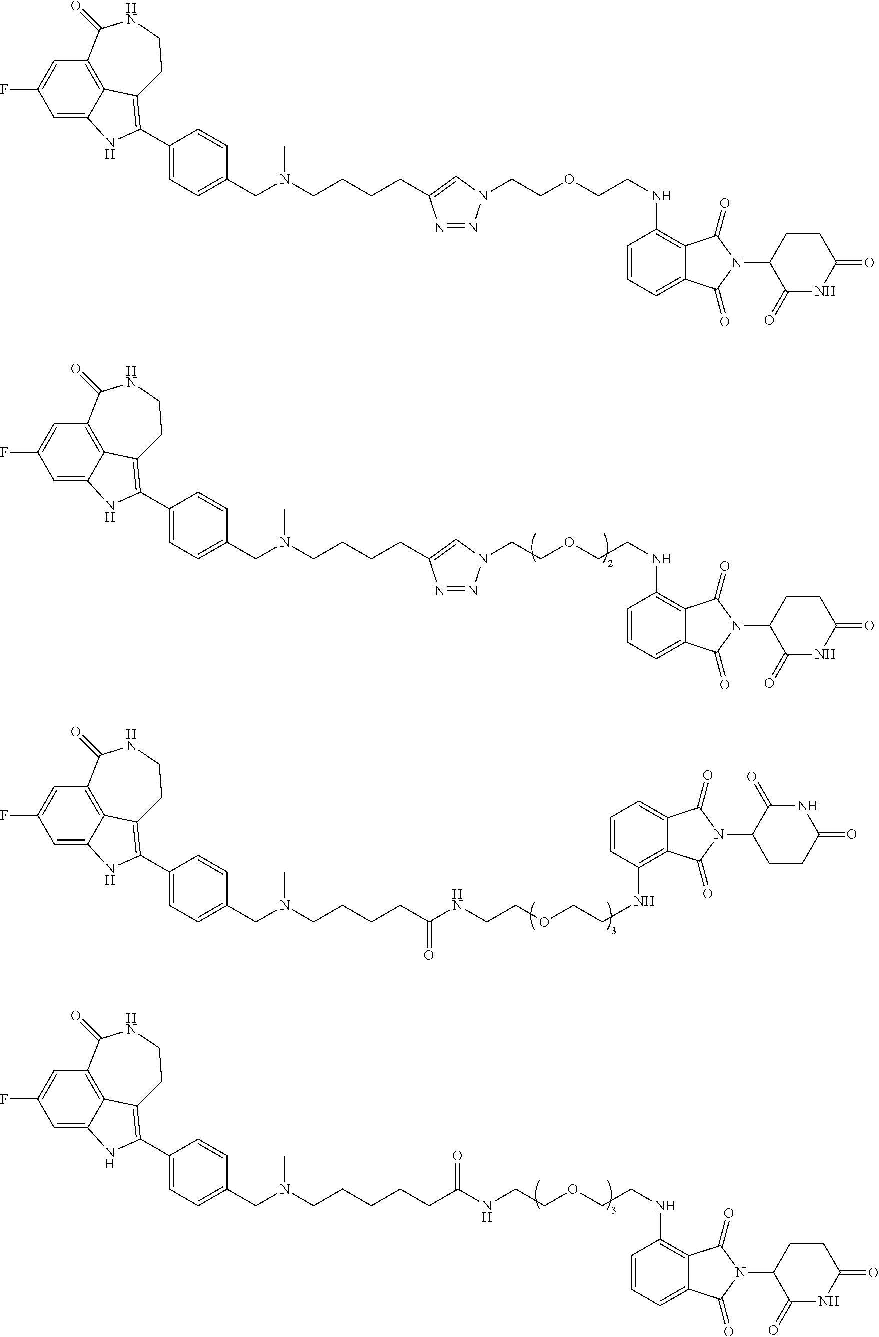

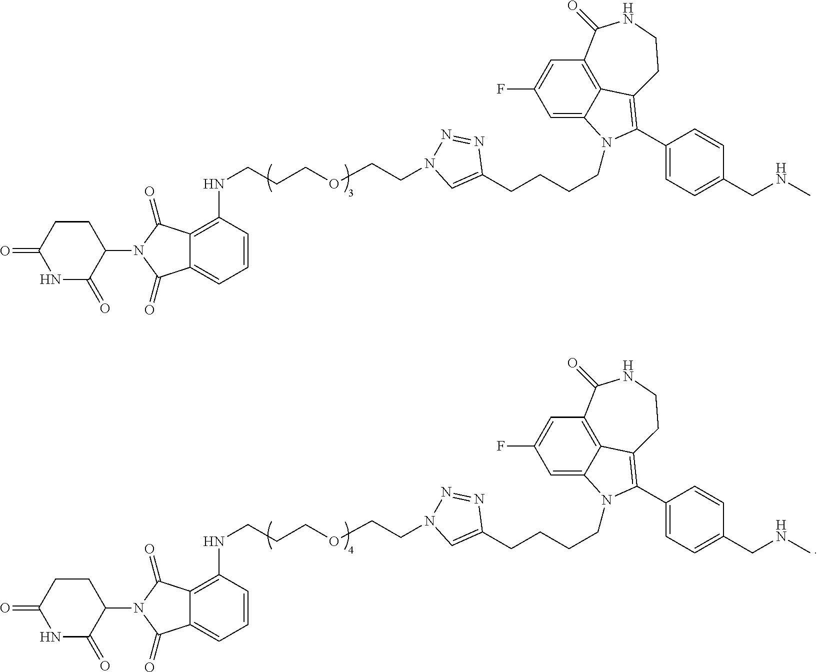

The compound may be selected from the group consisting of:

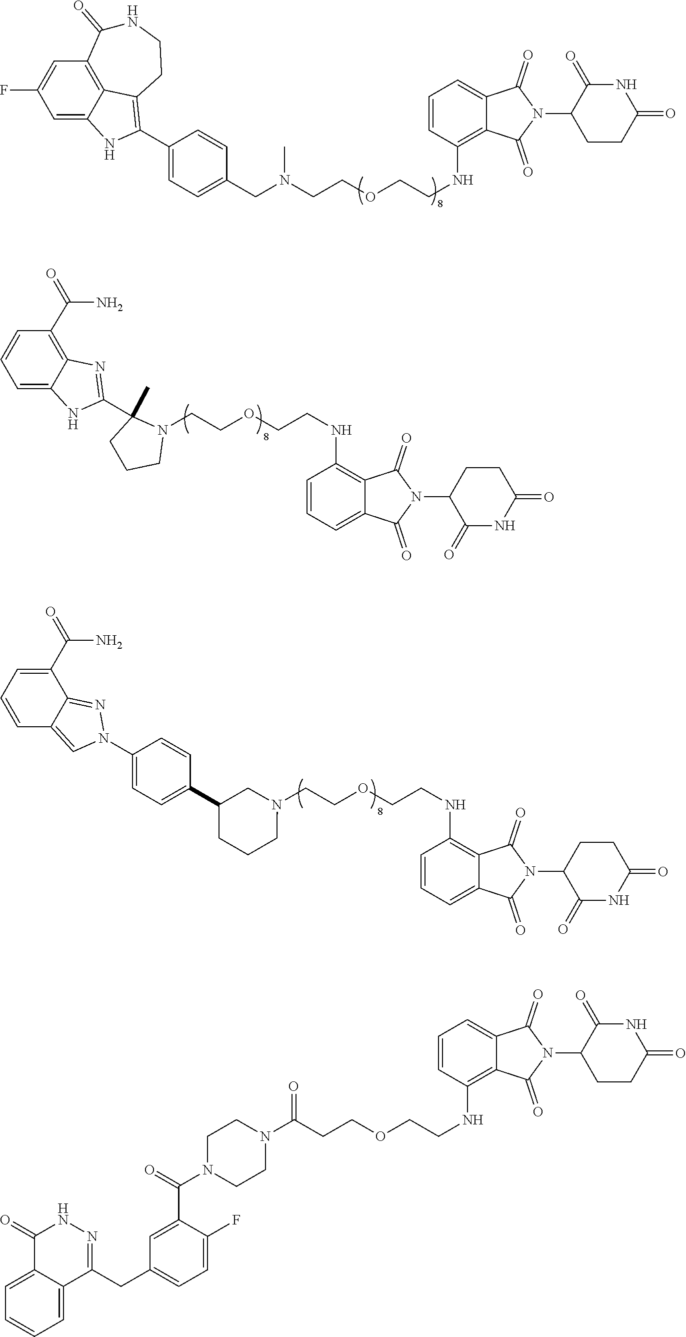

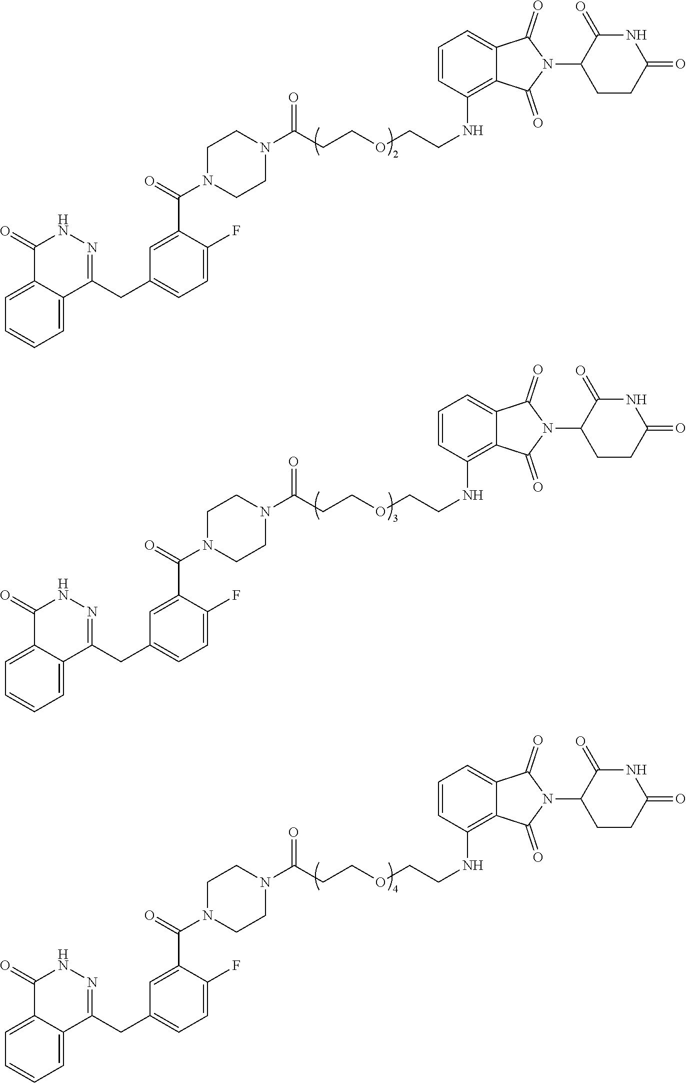

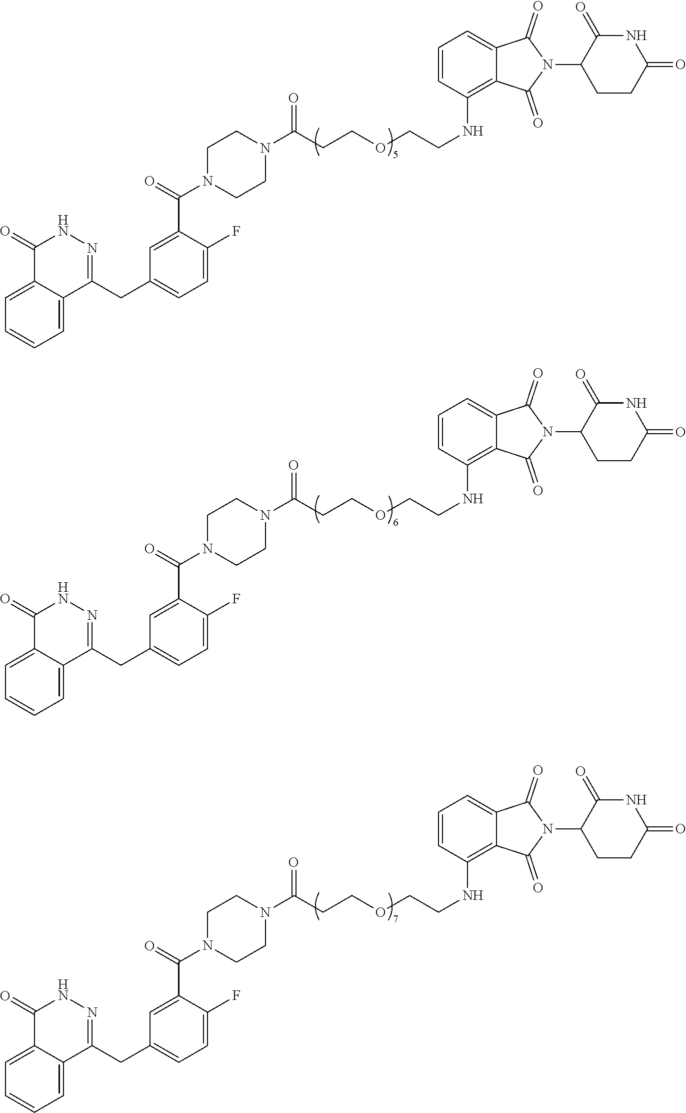

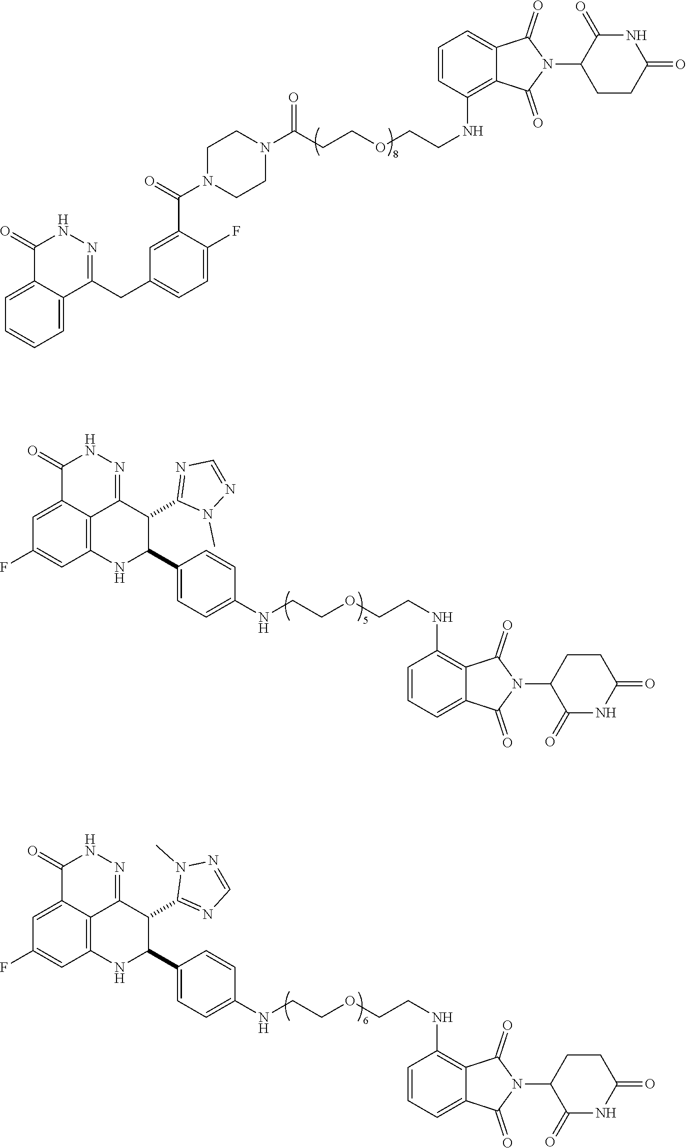

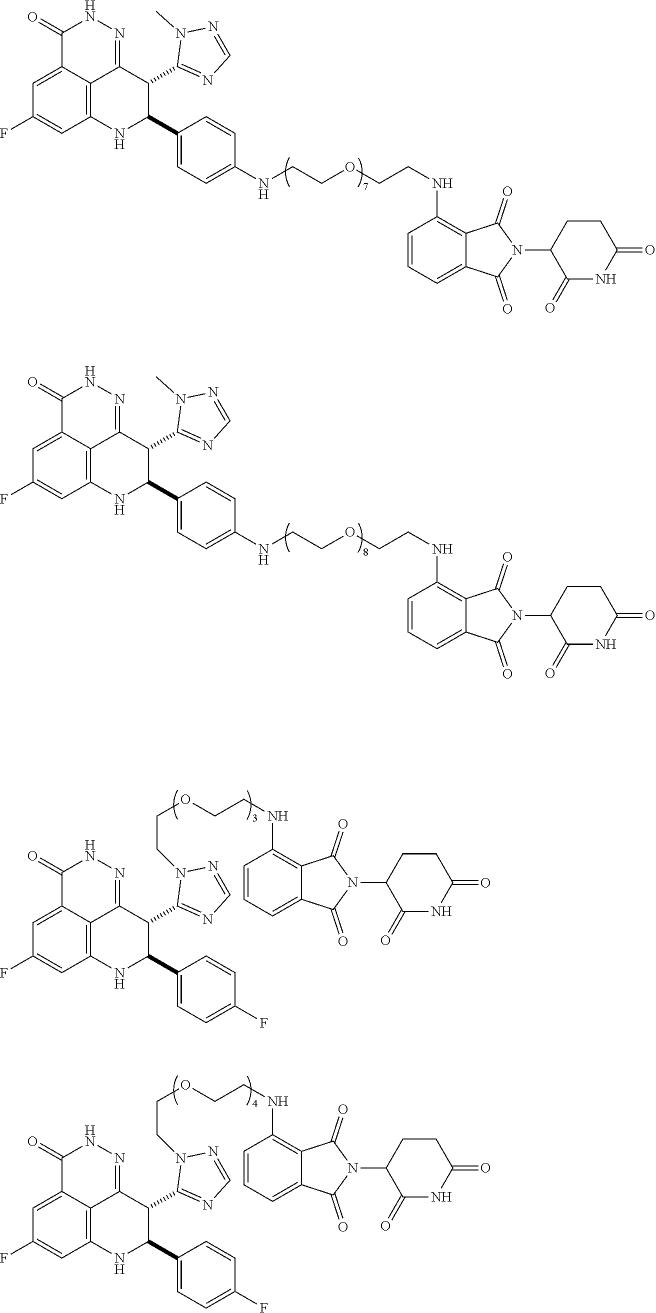

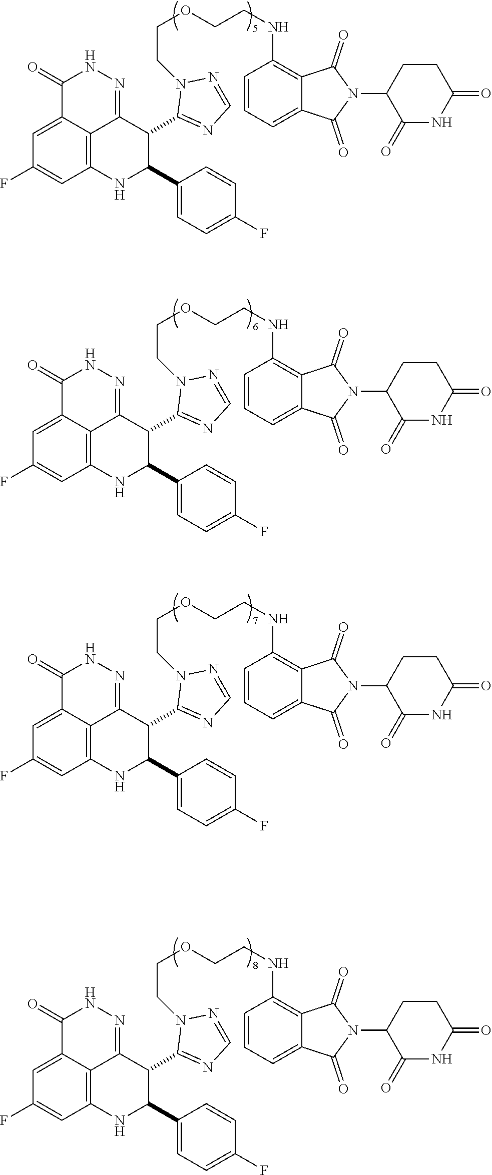

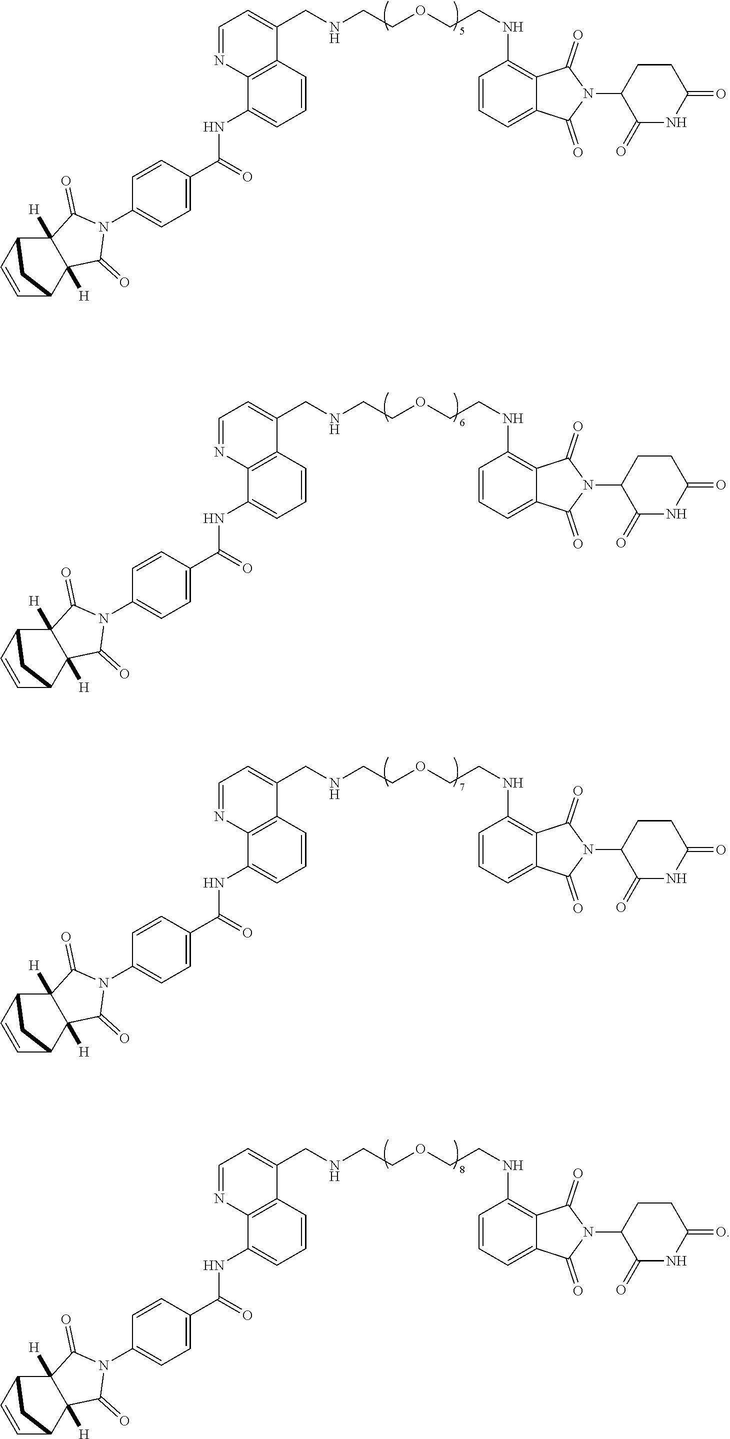

In another embodiment, there is provided a pharmaceutical composition comprising a compound as defined above and one or more pharmaceutically acceptable excipients.

In yet another embodiment, there is provided a pharmaceutical composition comprising a compound as defined above and at least one further therapeutic agent and one or more pharmaceutically acceptable excipients.

In still yet another embodiment, there is provided a method of inducing covalent modification of one or more surface-accessible lysine residues of a PARP protein comprising administering to a patient in need thereof, an effective amount of a compound as defined above, or a pharmaceutically acceptable salt thereof. The covalent modification may be is attachment of one or more ubiquitin molecules, of one or more SUMO molecules, of one or more ISG15 molecules, of one or more NEDD8 molecules, of one or more ATG8 molecules, of one or more ATG12 molecules, of one or more FAT10 molecules, of one or more HUB1 molecules, of one or more MNSFB molecules, of one or more UFM1 molecules, or of one or more URM1 molecules.

In a further embodiment, there is method of reducing the amount of one or more PARP proteins comprising administering to a patient in need thereof, an effective amount of a compound as defined above, or a pharmaceutically acceptable salt thereof.

In still a further embodiment, there is provided a method of modulating protein PARylation comprising administering to a patient in need thereof, an effective amount of a compound as defined above, or a pharmaceutically acceptable salt thereof.

In still yet a further embodiment, there is provided a method of treating a heart disease comprising administering to a patient in need thereof an effective amount of a compound as fined thereof, or a pharmaceutically acceptable salt thereof.

In an additional embodiment there is provided a method of treating ischemia-reperfusion injury comprising administering to a patient in need thereof an effective amount of a compound as defined above, or a pharmaceutically acceptable salt thereof. The ischemia-reperfusion injury may be due myocardial infarction, stroke, heart surgery, or trauma. The ischemia-reperfusion injury may be in the brain, heart, muscle, lung, kidney, liver, pancreas, or intestine. e

In yet an additional embodiment, there is provided a method of treating cancer comprising administering to a patient in need thereof an effective amount of a compound as defined above, or a pharmaceutically acceptable salt thereof. The cancer may be a solid cancer, such as lung cancer, prostate cancer, pancreatic cancer, liver cancer, brain cancer, ovarian cancer, uterine cancer, testicular cancer, breast cancer, endometrial cancer, skin cancer, head and neck cancer, stomach cancer, or colon cancer. The method may further comprise administering a second cancer therapy to said patient, such as a chemotherapy, a radiotherapy, an immunotherapy, a toxin therapy or surgery.

Still yet additional embodiments include a method of treating fibrosis comprising administering to a patient in need thereof an effective amount of a compound as defined above, or a pharmaceutically acceptable salt thereof, a method of treating metabolic syndrome comprising administering to a patient in need thereof an effective amount of a compound as defined above, or a pharmaceutically acceptable salt thereof, a method of treating diabetes comprising administering to a patient in need thereof an effective amount of a compound as defined above, or a pharmaceutically acceptable salt thereof, a method of treating a developmental disorder comprising administering to a patient in need thereof an effective amount of a compound as defined above, or a pharmaceutically acceptable salt thereof, a method of treating an inflammatory disease comprising administering to a patient in need thereof an effective amount of a compound as defined above, or a pharmaceutically acceptable salt thereof, a method of treating a neurological disorder comprising administering to a patient in need thereof an effective amount of a compound as defined above, or a pharmaceutically acceptable salt thereof, a method of treating septic shock comprising administering to a patient in need thereof an effective amount of a compound as defined above, or a pharmaceutically acceptable salt thereof, a method of treating acute pancreatitis comprising administering to a patient in need thereof an effective amount of a compound as defined above, or a pharmaceutically acceptable salt thereof, a method of treating acute lung injury comprising administering to a patient in need thereof an effective amount of a compound as defined above, or a pharmaceutically acceptable salt thereof, or a method of treating non-alcoholic fatty liver disease (NASH) comprising administering to a patient in need thereof an effective amount of a compound as defined above, or a pharmaceutically acceptable salt thereof.

The compound may be administered more than once for any of the foregoing methods, such as continuously over at least an hour or on a chronic basis.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by particular embodiments and optional features, modification and variation of the concepts herein disclosed may be resorted to by those skilled in the art, and that such modifications and variations are considered to be within the scope of this disclosure as defined by the appended claims.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIGS. 1A-K. iRucaparib induces PARP1 degradation. (FIG. 1A) The chemical structure of iRucaparib. The Rucaparib and pomalidomide moiety are highlighted in red and blue, respectively. (FIG. 1B) The human PARP1 catalytic domain in complex with Rucaparib (PDB: 4RV6). (FIGS. 1C-I) Degradation of PARP1 by iRucaparib in HeLa cells. (FIG. 1C) Cells were treated with increasing concentrations of iRucaparib for 24 hours. iRucap, iRucaparib. (FIG. 1D) Cells were treated with Rucaparib (5 μM), pomalidomide (5 μM), Rucaparib (5 μM) plus pomalidomide (5 μM), iRucaparib (5 μM), or iRucaparib (5 μM) plus Rucaparib (1 μM) or MG132 (1 μM) for 24 hours. Rucap/Ru, Rucaparib; Poma/Po, Pomalidomide; MG, MG-132. (FIG. 1E) Cells were treated with iRucaparib (5 μM) in the presence of increasing concentrations of pomalidomide for 24 hours. (FIG. 1F) Cells were pre-treated with iRucaparib (5 μM) for 12 hours followed by cycloheximide (10 μg/ml) treatment for the indicated times. CHX, Cycloheximide. (FIG. 1G) mRNA abundances of PARP1, PARP2 and PARP3 as determined by quantitative RT-PCR assays. Cells were treated with Rucaparib or iRucaparib (5 μM) for 24 hours (n=3, values represent mean±SEM). (FIG. 1H) Cells were treated with iRucaparib (5 μM) for the indicated times. (FIG. 1I) Cells were pre-treated with iRucaparib (5 μM) for 24 hours. iRucaparib was washed out for the indicated times. (FIG. 1J) PC-3 cells were treated with VHL-Rucaparib (10 μM) for the indicated times, or VHL-Rucaparib (10 μM) plus MG-132 (1 μM) for 24 hours. VHL-Rucap, VHL-Rucaparib. (FIG. 1K) BT-549 cells were treated with iRucaparib (5 μM) or VHL-Rucaparib (20 μM) for 24 hours. In these experiments, whole cell lysates were analyzed by immunoblotting assays using the indicated antibodies.

FIGS. 2A-I. iRucaparib selectively targets PARP1 for degradation. (FIGS. 2A-C) Reproducibility of the HeLa TMT experiments. The S/N values (signal-to-noise ratios) of the corresponding TMT channels for each protein were extracted and were Log 10-transformed for (FIG. 2A) control group (DMSO), (FIG. 2B) iRucaparib treatment group and (FIG. 2C) iRucaparib plus Rucaparib treatment group. (FIG. 2D) Identification of a PARP1 peptide (VVSEDFLQDVSASTK; SEQ ID NO: 1). (FIG. 2E) Comparison of protein expression between the iRucaparib treatment vs. DMSO control. The S/N values of each protein in the two biological replicate samples were summed, and the ratio was log 2-transformed. PARP1 (red dot) and ZFP91 (black dot) are indicated by the corresponding arrows. (FIG. 2F) Comparison of protein expression between the iRucaparib treatment vs. iRucaparib+Rucaparib treatment. The S/N values of each protein in the two biological replicate samples were summed, and the ratio was log 2-transformed. PARP1 (red dot) is indicated by an arrow. (FIG. 2G) Log-Log plot comparing protein expression in iRucaparib treatment vs. VHL-Rucaparib treatment in BT-549 cells. The corresponding ratio (compared to DMSO) was log 2-transformed. PARP1 (red dot) and ZFP91 (black dot) are indicated by the corresponding arrows. (FIG. 2H) Heatmap presentation of the protein expression changes in the HeLa TMT experiment. All data was normalized to the first control sample, which was then log 2-transformed. (FIG. 2I) The expression level of selected proteins as measured in the HeLa TMT experiments.

FIGS. 3A-D. iRucaparib inhibits the ADP-ribosylation-mediated signaling events downstream of PARP1. (FIG. 3A) Quantitative analyses of the D/E-ADP-ribosylated proteome. Both SILAC-labeled HeLa cells were pretreated with a PARG inhibitor (PDD 00017273, 2 μM) for one hour. Light cells and heavy cells were then treated with DMSO and Rucaparib (10 μM), respectively, for another hour. Both cells were then challenged with H₂O₂ (2 mM) for 5 min. Whole cells lysates were combined at a 1:1 ratio, and the PARylated peptides were enriched and analyzed by quantitative mass spectrometry. This experiment was then repeated for iRucaparib (10 μM). (FIG. 3B) Immunoblotting analysis of the PARylation level in HeLa cells treated with either Rucaparib or iRucaparib. Cells were treated as shown in FIG. 3A. (FIG. 3C) Identification of a Rucaparib-sensitive (upper panel) and iRucaparib-sensitive (lower panel) PARP1 auto-modified peptide (AEPVE*VVAPR; SEQ ID NO: 11). The site of modification is indicated by an asterisk. The inset shows α ˜1:1 ratio (heavy/light) of a non-PARylated peptide (HQSFVLVGETGSGK; SEQ ID NO: 2) from DHX15. The upper panel shows the peptides extracted from the Rucaparib-SILAC experiment, and the lower panel shows the peptides extracted from the iRucaparib-SILAC experiment. (FIG. 3D) Correlation analysis for the ADP-ribosylated peptides identified in the Rucaparib and iRucaparib SILAC experiments. Log 2(compound/control) values are shown (median values if identified multiple times).

FIGS. 4A-G. iRucaparib abrogates PARPi-induced PARP1 trapping. (FIG. 4A) PARP1 trapping in HeLa cells treated with Rucaparib or iRucaparib. Cells were pretreated with Rucaparib or iRucaparib (5 μM) for 24 hours followed by a 2-hour MMS (0.01%) treatment. Nuclear soluble and chromatin-bound proteins were extracted and analyzed. (FIG. 4B) Comparison of PARP1 trapping by BMN673, Niraparib, Olaparib, Rucaparib, Veliparib and iRucaparib in HeLa cells. The assay was performed as in FIG. 4A with a concentration of 5 μM for all compounds. (FIG. 4C) Cell cycle analysis of HeLa cells after Rucaparib or iRucaparib treatment. HeLa cells were treated with Rucaparib or iRucaparib (10 μM) for 48 hours, and then were analyzed by flow cytometry. The left and right peaks indicate G1 and G2/M populations, respectively, with the corresponding quantification results shown in FIG. 4D. (FIG. 4E) γH2A.X levels in HeLa cells after 48 hours of treatment with Rucaparib or iRucaparib (both at 10 μM). (FIG. 4F) Cell proliferation analyses of HeLa cells after the treatment of Rucaparib or iRucaparib. Cells were treated with Rucaparib or iRucaparib (both at 10 μM) for 72 hours. (FIG. 4G) Survival curve of HeLa cells treated with MMS alone or in combination with Rucaparib or iRucaparib (10 μM). Values represent mean±SEM (n=3).

FIGS. 5A-I. iRucaparib protects cells from genotoxicity-induced cell death. (FIG. 5A) iRucaparib blocks PARP1-induced PARylation. C2C12 myotubes were pretreated with Rucaparib or iRucaparib (5 μM) for 24 hours and were then treated with the PARG inhibitor PDD 00017273 (2 μM) for 1 hour. The cells were treated with MMS (0.01%) for 1 hr and were analyzed by immunoblotting assays. The asterisk indicates a non-specific band. (FIG. 5B) iRucaparib protects cells from MMS-induced NAD⁺ depletion. Cells were pretreated with Rucaparib or iRucaparib (5 μM) for 24 hours, which was followed by a 4-hour MMS (0.01%) treatment. The left and right panel shows the results for C2C12 myotubes and primary rat cardiomyocytes, respectively. (FIG. 5C) iRucaparib protects cells from MMS-induced ATP depletion. Cells were pretreated with Rucaparib or iRucaparib (5 μM) for 24 hours, which was followed by a 9-hour MMS (0.01%) treatment. ATP levels were determined using the CellTiter-Glo assay. The left and right panel shows the results for C2C12 myotubes and primary rat cardiomyocytes, respectively. (FIGS. 5D-E) PARP1 trapping as a result of the treatment of Rucaparib and iRucaparib in (FIG. 5D) C2C12 myotubes and (FIG. 5E) primary rat cardiomyocytes. Cells were pretreated with Rucaparib or iRucaparib (5 μM) for 24 hours, which was followed by a 2-hour treatment of MMS (0.01%). Chromatin-bound proteins were extracted and analyzed by immunoblotting experiments. (FIGS. 5F-G) DNA damage response as a result of the treatment of Rucaparib or iRucaparib in (FIG. 5F) C2C12 myoblasts and (FIG. 5G) primary rat cardiomyocytes. Cells were treated with Rucaparib or iRucaparib (10 μM) for 48 hours and were then analyzed by immunoblotting experiments. (FIG. 5H) DNA damage response (γH2A.X immunofluorescence) as a result of the treatment of Rucaparib or iRucaparib (10 μM) in C2C12 myoblasts. Scale bar=50 μm. (FIG. 5I) Cell growth analysis of C2C12 myoblasts treated with Rucaparib or iRucaparib. Cells were treated with Rucaparib or iRucaparib (5 μM) for 72 hours. Values represent mean±SEM (n=3).

FIG. 6. IWR-PROTAC compound induces PARP5A/B degradation in HEK293 cells whereas IWR-1 induces accumulation of PARP5A/B.

FIG. 7. Veliparib-PEG6-pomalidomide compound selectively induces PARP2 degradation in HeLa cells.

FIGS. 8A-E. The chemical structure of the PARP1 PROTACs (FIG. 8A) CRBN-based PARP1 PROTAC with a PEG1 linker. (FIG. 8B) CRBN-based PARP1 PROTAC with a PEG2 linker. (FIG. 8C) CRBN-based PARP1 PROTAC with a PEG3 linker. (FIG. 8D) CRBN-based PARP1 PROTAC with a PEG4 linker. (FIG. 8E) VHL-based PARP1 PROTAC with a PEG4 linker.

FIGS. 9A-G. PARP1 PROTAC compounds induce PARP1 degradation in various cell lines. (FIG. 9A) iRucaparib induces PARP1 degradation in MDA-MB468 cells. Cells were treated with increasing concentrations of iRucaparib. (FIGS. 9B-D) PARP1 degradation is affected by the linker lengths in the PROTAC compound. HeLa cells were treated with increasing concentrations of the CRBN-based PROTACs bearing a (FIG. 9B) PEG1 linker, (FIG. 9C) PEG2 linker or (FIG. 9D) PEG4 linker for 24 hours. (FIG. 9E) Expression of VHL and PARP1 in different cells. (FIGS. 9F-G) PARP1 degradation is regulated by the presence of the E3 ligase relevant to the PROTAC compound. Cells were treated with VHL-Rucaparib (10 μM) for the indicated times, or VHL-Rucaparib plus MG-132 (1 μM) for 24 hours in (FIG. 9F) BT-549 cells and (FIG. 9G) 786-O cells. VHL-Rucap, VHL-Rucaparib.

FIGS. 10A-B. iRucaparib specifically targets PARP1 for degradation. (FIG. 10A) iRucaparib induces PARP1 degradation, which is recused by Rucaparib treatment. HeLa cells were treated with iRucaparib (5 μM) or iRucaparib (5 μM) plus Rucaparib (1 μM) for 24 hours. Samples were subject to TMT-based quantitative proteomic analyses as shown in FIGS. 2A-i. (FIG. 10B) Comparison of protein expression between the iRucaparib+Rucaparib treatment vs. DMSO control. The S/N values of each protein in the two biological replicate samples were summed, and the ratio was log 2-transformed. ZFP91 (black dot) is indicated by the corresponding arrow.

FIG. 11. The catalytic inhibitory activity of Rucaparib and the CRBN-based PARP1 PROTACs. IC₅₀ values represent mean±SEM (n=3).

FIGS. 12A-E. iRucaparib prevents PARP1 trapping-induced cell toxicity. (FIGS. 12A-C) iRucaparib induces PARP1 degradation in (FIG. S5A) C2C12 myoblasts, (FIG. 12B) C2C12 myotubes and (FIG. 12C) Primary rat cardiomyocytes. Cells were treated with increasing concentrations of iRucaparib for 24 hours. (FIGS. S5D-E) iRucaparib treatment does not induce DNA damage response in cells. γH2A.X immunofluorescence levels in (FIG. 12D) C2C12 myotubes and (FIG. 12E) primary rat cardiomyocytes after the treatment with Rucaparib or iRucaparib (10 μM for 48 hours). Scale bar=50 μm.

FIG. 13. HEK293 cells treated with Wnt3A conditioned media for 24 h followed by IWR-1 or IWR-triazole-PEG4-pomalidomide for 24 h.

FIG. 14. HEK293 cells treated with Wnt3A conditioned media for 24 h followed by IWR-1 or IWR-PEG4-pomalidomide for 24 h.

FIG. 15. HEK293 cells treated with Wnt3A conditioned media for 24 h followed by IWR-1 or IWR-PEG4-VHL-ligand for 24 h.

FIG. 16. DLD-1 cells treated with IWR-1 or IWR-triazole-PEG4-pomalidomide for 24 h.

FIG. 17. DLD-1 cells treated with IWR-1 or IWR-PEG4-pomalidomide for 24 h.

FIG. 18. DLD-1 cells treated with IWR-1 or IWR-PEG4-VHL-ligand for 24 h.

### DETAILED DESCRIPTION

The inventors designed a series of PARP1 PROTACs in which they linked an FDA-approved PARP1 inhibitor (Rucaparib) to either CRBN or VHL binders. Although PARP1 is a highly abundant protein, one of such compounds, iRucaparib (a bivalent compound of Rucaparib conjugated to Pomalidomide via a PEG3 linker), induced potent and reversible degradation of PARP1 in a CRBN-dependent fashion. Treatment with iRucaparib resulted in selective degradation of PARP1 with limited effects on the protein level of other PARPs. This strategy allows the removal of PARP1, which enables, for the first time, pharmacological dissection of PARP1 inhibition vs PARP1 trapping in mediating cellular stress responses. Finally, iRucaparib is able to protect muscle cells and primary rat cardiomyocytes from DNA damage-induced PARP1 activation and energy depletion, without eliciting the deleterious effects associated with PARP1 trapping. Besides offering mechanistic insights into PARP1 trapping, these results suggest that PARP1 PROTACs represent a promising approach for the amelioration of the various pathological conditions caused by PARP1 hyperactivation. These and other aspects of the disclosure are set out in detail below.

### I. DEFINITIONS

In general, the terms and phrases used herein have their art-recognized meaning, which can be found by reference to standard texts, journal references and contexts known to those skilled in the art. Such art-recognized meanings may be obtained by reference to technical dictionaries, such as Hawley's Condensed Chemical Dictionary 14^{th} Edition, by R. J. Lewis, John Wiley & Sons, New York, N.Y., 2001.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

While the present invention can take many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications of the described embodiments and any further applications of the principles of the invention as described herein are contemplated as would normally occur to one skilled in the art to which the invention relates.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Whenever a range is given in the specification, for example, a temperature range, a time range, a carbon chain range, or a composition or concentration range, all intermediate ranges and subranges, as well as all individual values included in the ranges given are intended to be individually included in the disclosure. It will be understood that any subranges or individual values in a range or subrange that are included in the description can be optionally excluded from embodiments of the invention.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein, any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

A “chemotherapeutic agent” refers to any substance capable of reducing or preventing the growth, proliferation, or spread of a cancer cell, a population of cancer cells, tumor, or other malignant tissue. The term is intended also to encompass any antitumor or anticancer agent.

A “therapeutically effective amount” of a compound with respect to the subject method of treatment refers to an amount of the compound(s) in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate. The term “treating” or “treatment” can include reversing, reducing, or arresting the symptoms, clinical signs, and underlying pathology of a condition in manner to improve or stabilize a subject's condition.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

The term “exposing” is intended to encompass definitions as broadly understood in the art. In an embodiment, the term means to subject or allow to be subjected to an action, influence, or condition. For example, and by way of example only, a cell can be subjected to the action, influence, or condition of a therapeutically effective amount of a pharmaceutically acceptable form of a chemotherapeutic agent.

The term “cancer cell” is intended to encompass definitions as broadly understood in the art. In an embodiment, the term refers to an abnormally regulated cell that can contribute to a clinical condition of cancer in a human or animal. In an embodiment, the term can refer to a cultured cell line or a cell within or derived from a human or animal body. A cancer cell can be of a wide variety of differentiated cell, tissue, or organ types as is understood in the art.

The term “tumor” refers to a neoplasm, typically a mass that includes a plurality of aggregated malignant cells.

### II. PROTACs

A proteolysis targeting chimera (PROTAC) is a two-headed molecule capable of removing specific unwanted proteins. Rather than acting as a conventional enzyme inhibitor, a PROTAC works by inducing selective intracellular proteolysis. PROTACs consist of two covalently linked protein-binding molecules: one capable of engaging an E3 ubiquitin ligase, and another that binds to a target protein meant for degradation. Recruitment of the E3 ligase to the target protein results in ubiquitination and subsequent degradation of the target protein by the proteasome. Because PROTACs need only to bind their targets with high selectivity (rather than inhibit the target protein's enzymatic activity), there are currently many efforts to retool previously ineffective inhibitor molecules as PROTACs for next-generation drugs.

PROTAC technology is described in detail in U.S. Patent Publications 20180228907 and 20160272639, both of which are incorporated herein by reference.

### III. PARP PROTEINS AND PARP INHIBITION

A. PARP Proteins

Poly [ADP-ribose] polymerase 1 (PARP-1) also known as NAD⁺ ADP-ribosyltransferase 1 or poly[ADP-ribose] synthase 1 is an enzyme that in humans is encoded by the PARP1 gene. It is one of the PARP family of enzymes. PARP1 works by modifying nuclear proteins by poly ADP-ribosylation, and in conjunction with BRCA, which acts on double strands. Members of the PARP family act on single strands; or, when BRCA fails, PARP takes over those jobs as well (in a DNA repair context). PARP1 is involved in differentiation, proliferation, and tumor transformation, normal or abnormal recovery from DNA damage, the pathophysiology of type I diabetes, and it may be the site of mutation in Fanconi anemia. PARP1 is activated by Helicobacter pylori in the development and proliferation of gastric cancer.

PARP1 has a role in repair of single-stranded DNA (ssDNA) breaks. Knocking down intracellular PARP1 levels with siRNA or inhibiting PARP1 activity with small molecules reduces repair of ssDNA breaks. In the absence of PARP1, when these breaks are encountered during DNA replication, the replication fork stalls, and double-strand DNA (dsDNA) breaks accumulate. These dsDNA breaks are repaired via homologous recombination (HR) repair, a potentially error-free repair mechanism. For this reason, cells lacking PARP1 show a hyper-recombinagenic phenotype (e.g., an increased frequency of HR), which has also been observed in vivo in mice using the pun assay. Thus, if the HR pathway is functioning, PARP1 null mutants (cells without functioning PARP1) do not show an unhealthy phenotype, and in fact, PARP1 knockout mice show no negative phenotype and no increased incidence of tumor formation.

PARP1 is one of six enzymes required for the highly error-prone DNA repair pathway microhomology-mediated end joining (MMEJ). MMEJ is associated with frequent chromosome abnormalities such as deletions, translocations, inversions and other complex rearrangements. When PARP1 is up-regulated, MMEJ is increased, causing genome instability. PARP1 is up-regulated and MMEJ is increased in tyrosine kinase-activated leukemias.

PARP1 is also over-expressed when its promoter region ETS site is epigenetically hypomethylated, and this contributes to progression to endometrial cancer, BRCA-mutated ovarian cancer, and BRCA-mutated serous ovarian cancer.

PARP1 is also over-expressed in a number of other cancers, including neuroblastoma, HPV infected oropharyngeal carcinoma, testicular and other germ cell tumors, Ewing's sarcoma, malignant lymphoma, breast cancer, and colon cancer.

PARP5A/B (also known as tankyrase 1/2 or TNKS/TNKS2) control Wnt, YAP and AKT signaling, telomere maintenance, DNA repair, mitosis, and glucose metabolism (Haikrainen et al., 2014; Kim, 2018; Lüscher et al., 2018). Pharmacological inhibition of PARP5A/B is useful in a wide range of disease areas, including cancer, fibrosis, developmental disorders, and metabolic syndrome such as diabetes and fatty liver (Riffell et al., 2012; Lupo and Trusolino, 2014; Lum and Chen, 2015). However, despite intense efforts, currently there is no drug targeting PARP5A/B in clinical use. We have previously identified IWR-1 as the first-in-class small-molecule Wnt inhibitor (Chen et al., 2009) that works by antagonizing the PARP5A/B (Huang et al., 2009). In addition, inhibition of PARP5A/B induces telomeric damage and suppresses cancer cell immortalization (Kulak et al., 2015). However, pharmacological inhibition of PARP5A/B by small-molecules also prevents the autoregulation of PARP5A/B, leading to their accumulation and drug resistance (Rycker and Price, 2004). Additionally, PARP5A/B has scaffolding functions independent of their catalytic activities (Mariotti et al., 2017), making pharmaceutical inhibition of PARP5A/B insufficient to suppress their functions completely. Chemical knockdown of PARP5A/B by PROTAC represents a novel approach to modulate the activities of pathways controlled by PARP5A/B.

B. PARP Inhibitors

PARP inhibitors are a group of pharmacological inhibitors of the enzyme poly ADP ribose polymerase (PARP). They are developed for multiple indications; the most important is the treatment of cancer. Several forms of cancer are more dependent on PARP than regular cells, making PARP an attractive target for cancer therapy. PARP inhibitors appear to improve progression-free survival in women with recurrent platinum-sensitive ovarian cancer, as evidenced mainly by Olaparib added to conventional treatment.

In addition to their use in cancer therapy, PARP inhibitors are considered a potential treatment for acute life-threatening diseases, such as stroke and myocardial infarction, as well as for long-term neurodegenerative diseases.

DNA is damaged thousands of times during each cell cycle, and that damage must be repaired, including in cancer cells. Otherwise the cells may die due to this damage. BRCA1, BRCA2 and PALB2 are proteins that are important for the repair of double-strand DNA breaks by the error-free homologous recombinational repair, or HRR, pathway. When the gene for one of these proteins is mutated, the change can lead to errors in DNA repair that can eventually cause breast cancer. When subjected to enough damage at one time, the altered gene can cause the death of the cells.

PARP1 is a protein that is important for repairing single-strand breaks. If such nicks persist unrepaired until DNA is replicated (which must precede cell division), then the replication itself can cause double strand breaks to form. Drugs that inhibit PARP1 cause multiple double strand breaks to form in this way, and in tumors with BRCA1, BRCA2 or PALB2 mutations, these double strand breaks cannot be efficiently repaired, leading to the death of the cells. Normal cells that do not replicate their DNA as often as cancer cells, and that lack any mutated BRCA1 or BRCA2 still have homologous repair operating, which allows them to survive the inhibition of PARP1.

PARP1 inhibitors lead to trapping of PARP proteins on DNA in addition to blocking their catalytic action. This interferes with replication, causing cell death preferentially in cancer cells, which grow faster than non-cancerous cells. Some cancer cells that lack the tumor suppressor PTEN may be sensitive to PARP1 inhibitors because of downregulation of Rad51, a critical homologous recombination component, although other data suggest PTEN may not regulate Rad51. Hence PARP inhibitors may be effective against many PTEN-defective tumours (e.g., some aggressive prostate cancers). Cancer cells that are low in oxygen (e.g., in fast growing tumors) are sensitive to PARP inhibitors.

PARP1 inhibitors that are approved for marketing include Olaparib, Rucaparib, Niraparib and Talazoparib. Others in clinical trials are Veliparib, CEP 9722, E7016, BMN673 and BGB-290. See also U.S. Pat. No. 6,495,541, which is incorporated by reference.

IWR-1 and IWR-8 are highly selective PARP5A/B inhibitors that show little effects on other PARPs (Kulak et al., 2015). Structural studies indicate that IWR-1 exploits a histidine residue in the ADP-binding site unique to PARP5A/B and induces a conformation change in the D-loop region (Narwal et al., 2012; Gunaydin et al., 2012; Qiu et al., 2014). In contrast, XAV939 developed by Novartis and IWR-6 binds to the nicotinamide-binding site (Karlberg et al., 2010; Thorsell et al., 2017; Kirby et al., 2012) common to all PARPs and shows low inhibition selectivity. IWR-3 is a dual-pocket PARP5A/B inhibitor (Kulak et al., 2015). Currently, there are no PARP5A/B inhibitors in clinical use.

### IV. CEREBLON

Cereblon is a protein that in humans is encoded by the CRBN gene. The gene that encodes the cereblon protein is found on the human chromosome 3, on the short arm at position p26.3 from base pair 3,190,676 to base pair 3,221,394. CRBN orthologs are highly conserved from plants to humans.

It was believed that the drug thalidomide binds and inactivates cereblon, which leads to an antiproliferative effect on myeloma cells and a teratogenic effect on fetal development. Thalidomide was used as a treatment for morning sickness from 1957 until 1961 but was withdrawn from the market after it was discovered that it caused birth defects. It is estimated that 10,000 to 20,000 children were affected. However, the finding that cereblon inhibition is responsible for the teratogenic activity of thalidomide in the chick and zebrafish was cast into doubt due to a 2013 report that pomalidomide (a more potent thalidomide analog) does not cause teratogenic effects in these same model systems even though it is a stronger cereblon inhibitor than thalidomide is. Also, mutations in the CRBN gene are associated with autosomal recessive nonsyndromic intellectual disability, possibly as a result of dysregulation of calcium-activated potassium channels in the brain during development.

Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, this ubiquitination results in reduced levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein. Furthermore, cereblon and DDB2 bind to DDB1 in a competitive manner. Cereblon also binds to the large-conductance calcium-activated potassium channel (KCNMA1) and regulates its activity. Moreover, mice lacking this channel develop neurological disorders.

One particular inhibitor of cereblon is Pomalidomide (marketed as Pomalyst in the U.S. and Imnovid in the EU and Russia). This compound is a derivative of thalidomide marketed by Celgene. It is anti-angiogenic and also acts as an immunomodulator.

Pomalidomide was approved in February 2013 by the U.S. Food and Drug Administration (FDA) as a treatment for relapsed and refractory multiple myeloma. It has been approved for use in people who have received at least two prior therapies including lenalidomide and bortezomib and have demonstrated disease progression on or within 60 days of completion of the last therapy. It received a similar approval from the European Commission in August 2013.

The parent compound of pomalidomide, thalidomide, was originally discovered to inhibit angiogenesis in 1994. Based upon this discovery, thalidomide was taken into clinical trials for cancer, leading to its ultimate FDA approval for multiple myeloma. Structure-activity studies revealed that amino substituted thalidomide had improved antitumor activity, which was due to its ability to directly inhibit both the tumor cell and vascular compartments of myeloma cancers. This dual activity of pomalidomide makes it more efficacious than thalidomide in vitro and in vivo.

Clinical trials have been promising. Phase I trial results showed tolerable side effects. Phase II clinical trials for multiple myeloma and myelofibrosis reported ‘promising results’. Phase III results showed significant extension of progression-free survival, and overall survival (median 11.9 months vs. 7.8 months; p=0.0002) in patients taking pomalidomide and dexamethasone vs. dexamethasone alone.

Pomalidomide directly inhibits angiogenesis and myeloma cell growth. This dual effect is central to its activity in myeloma, rather than other pathways such as TNF alpha inhibition, since potent TNF inhibitors including rolipram and pentoxifylline do not inhibit myeloma cell growth or angiogenesis. Upregulation of interferon γ, IL-2 and IL-10 as well as downregulation of IL-6 have been reported for pomalidomide. These changes may contribute to pomalidomide's anti-angiogenic and anti-myeloma activities.

Like thalidomide, pomalidomide can cause harm to unborn babies when administered during pregnancy, so women taking pomalidomide must not become pregnant. To avoid embryo-fetal exposure, a “Risk Evaluation and Mitigation Strategy” (REMS) program was developed to ensure pregnancy prevention or distribution of the drug to those who are or might become pregnant. Women must produce two negative pregnancy tests and use contraception methods before beginning pomalidomide. Women must commit either to abstain continuously from heterosexual sexual intercourse or to use two methods of reliable birth control, beginning 4 weeks prior to initiating treatment with pomalidomide, during therapy, during dose interruptions and continuing for 4 weeks following discontinuation of pomalidomide therapy.

Pomalidomide is present in the semen of patients receiving the drug. Therefore, males must always use a latex or synthetic condom during any sexual contact with females of reproductive potential while taking pomalidomide and for up to 28 days after discontinuing pomalidomide, even if they have undergone a successful vasectomy. Male patients taking pomalidomide must not donate sperm.

See U.S. Pat. No. 5,635,517, which is incorporated by reference, for more disclosure relevant to pomalidomide.

### V. VON HIPPEL-LINDAU TUMOR SUPPRESSOR

The von Hippel-Lindau tumor suppressor also known as pVHL is a protein that in humans is encoded by the VHL gene. Mutations of the VHL gene are associated with von Hippel-Lindau disease. The VHL protein is a component of the protein complex that includes elongin B, elongin C, and cullin-2, and possesses ubiquitin ligase E3 activity. This complex is involved in the ubiquitination and degradation of a hypoxia-inducible factor (HIF), which is a transcription factor that plays a central role in the regulation of gene expression by oxygen. RNA polymerase II subunit POLR2G/RPB7 is also reported to be a target of this protein. Alternatively, spliced transcript variants encoding distinct isoforms have been observed.

Von Hippel-Lindau syndrome (VHL) is a dominantly inherited hereditary cancer syndrome predisposing to a variety of malignant and benign tumors of the eye, brain, spinal cord, kidney, pancreas, and adrenal glands. A germline mutation of this gene is the basis of familial inheritance of VHL syndrome. Individuals with VHL syndrome inherit one mutation in the VHL protein that causes the protein's normal function to be lost or altered. Over time, sporadic mutation in the second copy of the VHL protein can lead to carcinomas, in particular hemangioblastomas affecting the liver and kidneys, renal (and vaginal) clear cell adenocarcinomas. The disease is caused by mutations of the VHL gene on the short arm of the third chromosome (3p26-p25).

Under normal oxygen levels, HIF1α binds pVHL through 2 hydroxylated proline residues and is polyubiquitinated by pVHL. This leads to its degradation via the proteasome. During hypoxia, the proline residues are not hydroxylated and pVHL cannot bind. HIF1α causes the transcription of genes that contain the hypoxia response element. In VHL disease, genetic mutations cause alterations to the pVHL protein, usually to the HIF1α binding site.

The resultant protein is produced in two forms, an 18 kDa and a 30 kDa protein that functions as a tumor suppressor. The main action of the VHL protein is thought to be its E3 ubiquitin ligase activity that results in specific target proteins being ‘marked’ for degradation.

The most researched of these targets is hypoxia inducible factor 1a (HIF1a), a transcription factor that induces the expression of a number of angiogenesis related factors.

HIF is necessary for tumor growth because most cancers demand high metabolic activity and are only supplied by structurally or functionally inadequate vasculature.

Activation of HIF allows for enhanced angiogenesis, which in turn allows for increased glucose uptake. While HIF is mostly active in hypoxic conditions, VHL-defective renal carcinoma cells show constitutive activation of HIF even in oxygenated environments.

It is clear that VHL and HIF interact closely. Firstly, all renal cell carcinoma mutations in VHL that have been tested affect the protein's ability to modify HIF. Additionally, HIF activation can be detected in the earliest events in tumorigenesis in patients with VHL syndrome. In normal cells in hypoxic conditions, HIF1A is activated with little activation of HIF2A. However, in tumors the balance of HIF1A and HIF2A is tipped towards HIF2A. While HIF1A serves as a pro-apoptotic factor, HIF2A interacts with cyclin D1. This leads to increased survival due to lower rates of apoptosis and increased proliferation due to the activation of cyclin D1. Recent genome wide analysis of HIF binding in kidney cancer showed that HIF1A binds upstream of majorly good prognosis genes, while HIF2A binds upstream to majorly poor prognosis genes. This indicates that the HIF transcription factor distribution in kidney cancer is of major importance in determining the outcome of the patients.

In the normal cell with active VHL protein, HIF alpha is regulated by hydroxylation in the presence of oxygen. When iron, 2-oxoglutarate and oxygen are present, HIF is inactivated by HIF hydroxylases. Hydroxylation of HIF creates a binding site for pVHL (the protein product of the VHL gene). pVHL directs the polyubiquitylation of HIF1A, ensuring that this protein will be degraded by the proteasome. In hypoxic conditions, HIF1A subunits accumulate and bind to HIFB. This heterodimer of HIF is a transcription factor that activates genes that encode for proteins such as vascular endothelial growth factor (VEGF) and erythropoietin, proteins that are both involved in angiogenesis. Cells with abnormal pVHL are unable to disrupt the formation of these dimers, and therefore behave like they are hypoxic even in oxygenated environments.

HIF has also been linked to mTOR, a central controller of growth decisions. It has recently been shown that HIF activation can inactivate mTOR.

HIF can help explain the organ specific nature of VHL syndrome. It has been theorized that constitutively activating HIF in any cell could lead to cancer, but that there are redundant regulators of HIF in organs not affected by VHL syndrome. This theory has been disproved multiple times since in all cell types loss of VHL function leads to constitutive activation of HIF and its downstream effects. Another theory holds that although loss of VHL leads to activation of HIF in all cells, in most cells this leads to no advantage in proliferation or survival. Additionally, the nature of the mutation in the VHL protein leads to phenotypic manifestations in the pattern of cancer that develops. Nonsense or deletion mutations of VHL protein have been linked to type 1 VHL with a low risk of pheochromocytoma (adrenal gland tumors). Type 2 VHL has been linked to missense mutations and is linked to a high risk of pheochromocytoma. Type 2 has also been further subdivided based on risks of renal cell carcinoma. In types 1, 2A and 2B the mutant pVHL is defective in HIF regulation, while type 2C mutant are defective in protein kinase C regulation. These genotype-phenotype correlations suggest that missense mutations of pVHL lead to a ‘gain of function’ protein.

The involvement in VHL in renal cell cancer can be rationalized via multiple characteristics of renal cells. First, they are more sensitive to the effects of growth factors created downstream of HIF activation than other cells. Secondly, the link to Cyclin D1 (as mentioned above) is only seen in renal cells. Finally, many cells in the kidney normally operate under hypoxic conditions. This may give them a proliferative advantage over other cells while in hypoxic environments.

In addition to its interaction with HIF, the VHL protein can also associate with tubulin. It is then capable to stabilize and thus elongate microtubules. This function plays a key role in the stabilisation of the spindle during mitosis. Deletion of VHL causes a drastic increase of misorientated and rotating spindles during mitosis. Through a not yet known mechanism, VHL also increases the concentration of MAD2, an important protein of the spindle checkpoint. Thus VHL-loss leads to a weakened checkpoint and subsequently chromosome missegregation and aneuploidy.

The loss of VHL protein activity results in an increased amount of HIF1a, and thus increased levels of angiogenic factors, including VEGF and PDGF. In turn, this leads to unregulated blood vessel growth, one of the prerequisites of a tumor. Additionally, VHL has been implicated in maintaining the differentiated phenotype in renal cells. Furthermore, cell culture experiments with VHL −/− cells have shown that the addition of pVHL can induce a mesenchymal to epithelial transition. This evidence suggests that VHL has a central role in maintaining a differentiated phenotype in the cell.

Additionally, pVHL is important for extracellular matrix formation. This protein may also be important in inhibition of matrix metalloproteinases. These ideas are extremely important in the metastasis of VHL-deficient cells. In classical VHL disease a single wild-type allele in VHL appears to be sufficient to maintain normal cardiopulmonary function.

Suggested targets for VHL-related cancers include targets of the HIF pathway, such as VEGF. Inhibitors of VEGF receptor sorafenib, sunitinib, pazopanib, and recently axitinib have been approved by the FDA. The mTOR inhibitor rapamycin analogs everolimus and temsirolimus or VEGF monoclonal antibody bevacizumab may also be an option.

Since iron, 2-oxoglutarate and oxygen are necessary for the inactivation of HIF, it has been theorized that a lack of these cofactors could reduce the ability of hydroxlases in inactivating HIF. A recent study has shown that in cells with a high activation of HIF even in oxygenated environments was reversed by supplying the cells with ascorbate. Thus, Vitamin C may be a potential treatment for HIF induced tumors.

### VI. PHARMACEUTICAL COMPOSITIONS AND METHODS OF TREATMENT

A. Pharmaceutical Compositions and Routes of Administration

The exact formulation, route of administration and dosage for the compositions disclosed herein can be chosen by an individual physician or clinician in view of a patient's condition (see e.g., Fingl et al., in The Pharmacological Basis of Therapeutics, 1975, Ch. 1). It should be noted that the attending physician would know how to and when to terminate, interrupt, or adjust administration due to toxicity, or to organ dysfunctions, etc. Conversely, the attending physician would also know to adjust treatment to higher levels if the clinical response were not adequate (in light of or precluding toxicity aspects). The magnitude of an administered dose in the management of the disorder of interest can vary with the severity of the condition to be treated and to the route of administration. The severity of the condition may, for example, be evaluated, in part, by standard prognostic evaluation methods. Further, the dose and perhaps dose frequency, can also vary according to circumstances, e.g., the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995) and elsewhere in the art.

The compounds can be administered to a patient in combination with a pharmaceutically acceptable carrier, diluent, or excipient. The phrase “pharmaceutically acceptable” refers to those ligands, materials, compositions, and/or dosage forms that are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” includes any and all solvents, dispersion media, diluents, coatings, surfactants, antioxidants, preservatives (e.g., antibacterial agents, antifungal agents), isotonic agents, absorption delaying agents, salts, buffers, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the chemotherapeutic or pharmaceutical compositions is contemplated.

A PROTAC may be combined with different types of carriers depending on whether it is to be administered in solid, liquid or aerosol form, and whether it need to be sterile for such routes of administration as injection. The present disclosure can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present disclosure administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

When administered to a subject, effective amounts will depend, of course, on the particular cancer being treated; the genotype of the specific cancer; the severity of the cancer; individual patient parameters including age, physical condition, size and weight, concurrent treatment, frequency of treatment, and the mode of administration. These factors are well known to the physician and can be addressed with no more than routine experimentation. In some embodiments, it is preferred to use the highest safe dose according to sound medical judgment.

In certain embodiments, pharmaceutical compositions may comprise, for example, at least about 0.1% of a PROTAC. In other embodiments, the active compound may comprise between about 2% to about 75% of the weight of the unit, or between about 25% to about 60%, for example, and any range derivable therein. In other non-limiting examples, a dose may also comprise from about 0.1 mg/kg/body weight, 0.5 mg/kg/body weight, 1 mg/kg/body weight, about 5 mg/kg/body weight, about 10 mg/kg/body weight, about 20 mg/kg/body weight, about 30 mg/kg/body weight, about 40 mg/kg/body weight, about 50 mg/kg/body weight, about 75 mg/kg/body weight, about 100 mg/kg/body weight, about 200 mg/kg/body weight, about 350 mg/kg/body weight, about 500 mg/kg/body weight, about 750 mg/kg/body weight, to about 1000 mg/kg/body weight or more per administration, and any range derivable therein. In non-limiting examples of a derivable range from the numbers listed herein, a range of about 10 mg/kg/body weight to about 100 mg/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including, but not limited to parabens (e.g., methylparabens, propylparabens), chlorobutanol, phenol, sorbic acid, thimerosal or combinations thereof.

PROTACs as described herein may be formulated into a composition in a free base, neutral or salt form. Pharmaceutically acceptable salts include the salts formed with the free carboxyl groups derived from inorganic bases such as for example, sodium, potassium, ammonium, calcium or ferric hydroxides; or such organic bases as isopropylamine, triethylamine, histidine or procaine.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are optionally provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

In embodiments where the composition is in a liquid form, a carrier can be a solvent or dispersion medium comprising, but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose (HPC); or combinations thereof such methods. In many cases, it will be preferable to include isotonic agents, such as, for example, sugars, sodium chloride or combinations thereof.

Sterile injectable solutions are prepared by incorporating the active compounds in the required amount of the appropriate solvent with various other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose.

The composition should be stable under the conditions of manufacture and storage, and preserved against the contaminating action of microorganisms, such as bacteria and fungi. Thus, preferred compositions have a pH greater than about 5, preferably from about 5 to about 8, more preferably from about 5 to about 7. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin or combinations thereof.

B. Treatments

i. Cancer

The disclosure also provides methods of treating a patient that has cancer, such as a solid tumor. The disclosure further provides methods of inhibiting a tumor cell comprising exposing the tumor cell to a therapeutically effective amount of a PROTAC as described herein, wherein the tumor cell is treated, killed, or otherwise inhibited from growing. The tumor or tumor cells can be malignant tumor cells.

The methods of the disclosure may be thus used for the treatment or prevention of various neoplasia disorders including acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangiocarcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor. Accordingly, the compositions and methods described herein can be used to treat bladder cancer, brain cancer (including intracranial neoplasms such as glioma, meninigioma, neurinoma, and adenoma), breast cancer, colon cancer, lung cancer (SCLC or NSCLC) ovarian cancer, pancreatic cancer, and prostate cancer.

ii. Ischemia-Reperfusion

Ischemia-reperfusion injury is caused at least in part by the inflammatory response of damaged tissues. White blood cells carried to the area by the newly returning blood release a host of inflammatory factors such as interleukins as well as free radicals in response to tissue damage. The restored blood flow reintroduces oxygen within cells that damages cellular proteins, DNA and the plasma membrane. Damage to the cell's membrane may in turn cause the release of more free radicals. Such reactive species may also act indirectly in redox signaling to turn on apoptosis. Leukocytes may also build up in small capillaries, obstructing them and leading to more ischemia.

Myocardial infarction (MI), occurs when the blood supply to part of the heart is interrupted. This is most commonly due to occlusion (blockage) of a coronary artery following the rupture of a vulnerable atherosclerotic plaque, which is an unstable collection of lipids (like cholesterol) and white blood cells (especially macrophages) in the wall of an artery. The resulting ischemia (restriction in blood supply) and oxygen shortage, if left untreated for a sufficient period, can cause damage and/or death (infarction) of heart muscle tissue (myocardium). However, restoration of blood flow (reperfusion) may also cause damage.

Reperfusion injury also plays a part in the brain's ischemic cascade, which is involved in stroke and brain trauma. Repeated bouts of ischemia and reperfusion injury also are thought to be a factor leading to the formation and failure to heal of chronic wounds such as pressure sores and diabetic foot ulcers. Continuous pressure limits blood supply and causes ischemia, and the inflammation occurs during reperfusion. As this process is repeated, it eventually damages tissue enough to cause a wound.

A stenosis—an abnormal narrowing in a blood vessel or other tubular organ or structure—is one type of ischemic injury. Stenoses of the vascular type are often associated with a noise (bruit) resulting from turbulent flow over the narrowed blood vessel. This bruit can be made audible by a stethoscope. Other, more reliable methods of diagnosing a stenosis are imaging methods including ultrasound, Magnetic Resonance Imaging/Magnetic Resonance Angiography, Computed Tomography/CT-Angiography which combine anatomic imaging (i.e., the visible narrowing of a vessel) with the display of flow phenomena (visualization of the movement of the bodily fluid through the bodily structure). Vascular stenoses include intermittent claudication (peripheral artery stenosis), angina (coronary artery stenosis), carotid artery stenosis which predispose to (strokes and transient ischemic episodes) and renal artery stenosis.

The present disclosure contemplates treating individuals at risk for ischemia-reperfusion injury. These individuals would include those persons suffering from fibrosis. hypertension, cardiac hypertrophy, osteoporosis, neurodegeneration, and/or respiratory distress.

iii. Neurodegeneration

Besides regulating DDR in the context of human malignancies, poly-ADP-ribose is known to be a death signal, and PARP1 has been recently proposed as a promising therapeutic target for neurodegenerative diseases (e.g., Alzheimer's disease/AD and Parkinson's disease/PD). PARP1 has been shown to be directly activated by pathologic protein aggregates (e.g., α-synuclein and amyloid-β peptides) as well as many other neurotoxic stimuli (e.g., glutamate and H₂O₂) (Kam et al., 2018). Several of these agents activate nitric oxide synthase, leading to the generation of reactive oxygen species, and subsequently, DNA damage and PARP1 activation. Indeed, higher levels of PAR have been found in the brains and CSF (cerebrospinal fluid) of AD/PD patients (Kam et al., 2018). Importantly, genetic deletion or pharmacological inhibition of PARP1 offers profound protection against brain dysfunction in a large variety of animal models of neurodegenerative diseases (Dawson et al., 2017).

iv. Traumatic Brain Injury (TBI)

Traumatic brain injury usually results from a violent blow or jolt to the head or body. TBI can also be caused by an object that penetrates brain tissue, such as a bullet or shattered piece of skull that ultimately leads to brain cell death. Common events that cause TBI include: falls, vehicle related collisions, violence, sports injuries, explosive blast or other combat injuries. TBI can result in bruising, torn tissues, bleeding and other physical damage to the brain, which could lead to long-term complications or death. Symptoms could include loss of consciousness, repeated vomiting, seizures, loss of coordination, profound confusion, slurred speech. Research studies have suggested that repeated or severe TBI might increase the risk of neurodegenerative diseases, including Alzheimer's disease/AD and Parkinson's disease/PD. PARP1 is an important therapeutic target for TBI by preventing brain cell death.

v. Septic Shock

Septic shock is a severe medical condition that occurs when sepsis, which is organ injury or damage in response to infection, leads to dangerously low blood pressure and abnormalities in cellular metabolism. Septic shock is usually caused by infectious agents, including bacteria, fungi, viruses or parasites that are present in any part of body. Septic shock is a potentially fatal condition that causes multiple organ dysfunction. PARP1 is an important therapeutic target for septic shock by preventing cell death.

vi. Acute Pancreatitis

Acute pancreatitis is inflammation of the pancreas that occurs suddenly and produces severe upper abdominal pain, nausea and vomiting. A number of organs could be potentially affected, including kidneys, heart and lungs. Acute pancreatitis could cause life-threatening complications. Acute pancreatitis could be caused by a number of factors, including medications, structural abnormalities of pancreas, genetic mutations, metabolic conditions, traumatic injury, pancreatic cancer, among others.

vii. Acute Lung Injury

Acute lung injury (ALI) describes a characteristic form of parenchymal lung disease. ALI represents a wide range of severity from short-lived dyspnoea to a rapidly terminal failure of the respiratory system. Many factors could lead to ALI and the lung reacts to various types of insults in similar ways, regardless of etiology. The resultant endothelial and alveolar epithelial cell injury is attended by fluid and cellular exudation, which could lead to subsequent reparative fibroblastic proliferation and pneumocyte hyperplasia.

viii. Autoimmune Diseases

An autoimmune disease is a collective term for conditions in which your body is attacked by your own immune system. Most common autoimmune diseases include asthma, rheumatoid arthritis, lupus, celiac disease, Sjogren's syndrome, multiple sclerosis, type 1 diabetes, vasculitis, etc.

C. Combination Therapy

PROTACs as described herein can also be used in combination with other active ingredients. Such combinations are selected based on the condition to be treated, cross-reactivities of ingredients and pharmaco-properties of the combination. For example, when treating cancer, the compositions can be combined with other anti-cancer compounds (such as paclitaxel or rapamycin).

It is also possible to combine a compound of the disclosure with one or more other active ingredients in a unitary dosage form for simultaneous or sequential administration to a patient. The combination therapy may be administered as a simultaneous or sequential regimen. When administered sequentially, the combination may be administered in two or more administrations.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g. in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together. For example, a synergistic anti-cancer effect denotes an anti-cancer effect that is greater than the predicted purely additive effects of the individual compounds of the combination.

Accordingly, it is an aspect of this disclosure that a PROTAC can be used in combination with another agent or therapy method, preferably another cancer treatment. A PROTAC may precede or follow the other agent treatment by intervals ranging from minutes to weeks. In embodiments where the other agent and expression construct are applied separately to the cell, one would generally ensure that a significant period of time did not elapse between the time of each delivery, such that the agent and expression construct would still be able to exert an advantageously combined effect on the cell. For example, in such instances, it is contemplated that one may contact the cell, tissue or organism with two, three, four or more modalities substantially simultaneously (i.e., within less than about a minute) with the active agent(s). In other aspects, one or more agents may be administered within about 1 minute, about 5 minutes, about 10 minutes, about 20 minutes about 30 minutes, about 45 minutes, about 60 minutes, about 2 hours, about 3 hours, about 4 hours, about 6 hours, about 8 hours, about 9 hours, about 12 hours, about 15 hours, about 18 hours, about 21 hours, about 24 hours, about 28 hours, about 31 hours, about 35 hours, about 38 hours, about 42 hours, about 45 hours, to about 48 hours or more prior to and/or after administering the active agent(s). In certain other embodiments, an agent may be administered within from about 1 day, about 2 days, about 3 days, about 4 days, about 5 days, about 6 days, about 8 days, about 9 days, about 12 days, about 15 days, about 16 days, about 18 days, about 20 days, to about 21 days prior to and/or after administering the active agent(s). In some situations, it may be desirable to extend the time period for treatment significantly, however, where several weeks (e.g., about 1, about 2, about 3, about 4, about 6, or about 8 weeks or more) lapse between the respective administrations.

Administration of the chemotherapeutic compositions of the present disclosure to a patient will follow general protocols for the administration of chemotherapeutics, taking into account the toxicity, if any. It is expected that the treatment cycles would be repeated as necessary. It also is contemplated that various standard therapies or adjunct cancer therapies, as well as surgical intervention, may be applied in combination with the described active agent(s). These therapies include but are not limited to chemotherapy, radiotherapy, immunotherapy, gene therapy and surgery.

i. Chemotherapy

Cancer therapies can also include a variety of combination therapies with both chemical and radiation-based treatments. Combination chemotherapies include the use of chemotherapeutic agents such as, cisplatin, etoposide, irinotecan, camptostar, topotecan, paclitaxel, docetaxel, epothilones, taxotere, tamoxifen, 5-fluorouracil, methoxtrexate, temozolomide, cyclophosphamide, SCH 66336, R115777, L778,123, BMS 214662, IRESSA™ (gefitinib), TARCEVA™ (erlotinib hydrochloride), antibodies to EGFR, GLEEVEC™ (imatinib), intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatine, vinblastine, vincristine, vindesine, bleomycin, doxorubicin, dactinomycin, daunorubicin, epirubicin, idarubicin, mithramycin, deoxycoformycin, Mitomycin-C, L-Asparaginase, teniposide, 17α-Ethinylestradiol, Diethylstilbestrol, testosterone, prednisone, fluoxymesterone, dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesterone acetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, Avastin, herceptin, Bexxar, Velcade, Zevalin, Trisenox, Xeloda, Vinorelbine, Porfimer, Erbitux™ (cetuximab), Liposomal, Thiotepa, Altretamine, Melphalan, Trastuzumab, Lerozole, Fulvestrant, Exemestane, Fulvestrant, Ifosfomide, Rituximab, C225, Campath, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP 16), tamoxifen, raloxifene, estrogen receptor binding agents, paclitaxel, gemcitabine, navelbine, farnesyl-protein transferase inhibitors, transplatinum, 5-fluorouracil, vincristine, vinblastine and methotrexate, or any analog or derivative variant of the foregoing.

ii. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (e.g., 3 to 4 wks), to single doses of 2000 to 6000 roentgens. Dosage ranges for radioisotopes vary widely, and depend on the half-life of the isotope, the strength and type of radiation emitted, and the uptake by the neoplastic cells. The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

iii. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionucleotide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p¹55.

iv. Gene Therapy

In yet another embodiment, the secondary treatment is a secondary gene therapy in which a therapeutic polynucleotide is administered before, after, or at the same time a first chemotherapeutic agent. Delivery of the chemotherapeutic agent in conjunction with a vector encoding a gene product will have a combined anti-hyperproliferative effect on target tissues.

v. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies. Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

### VI. EXAMPLES

The following Examples are intended to illustrate the above disclosure and should not be construed as to narrow its scope. One skilled in the art will readily recognize that the Examples suggest many other ways in which the disclosure could be practiced. It should be understood that numerous variations and modifications may be made while remaining within the scope of the disclosure. The disclosure may be further understood by the following non-limiting examples.

### Example 1—Materials and Methods

Chemicals and Reagents. Olaparib was purchased from LC laboratory. BMN-673, Veliparib, Niraparib, Rucaparib, MG-132 and Pomalidomide were obtained from Selleck. PDD 00017273 was purchased from Tocris. Antibodies against GAPDH and Poly-(ADP-Ribose) were purchased from Thermo and Trevigen, respectively. Antibodies against PARP1, SP1, H3, γH2A.X and VHL were purchased from Cell Signaling Technology. All other chemicals and reagents were obtained from Sigma, unless stated otherwise.

Cell Culture. All the cells were purchased from ATCC. HeLa cells, murine C2C12 myoblasts and MDA-MB-468 cells were maintained in high glucose DMEM medium supplemented with 10% fetal bovine serum. C2C12 myoblasts were differentiated into myotubes using high glucose DMEM supplemented with 2% horse serum Pirinen et al., (2014). 786-O cells and PC-3 cells were maintained in RPMI-1640 medium supplemented with 10% fetal bovine serum. BT-549 cells were maintained in RPMI-1640 medium supplemented with 10% fetal bovine serum and 1 μg/mL insulin. Neonatal rat cardiomyocytes were isolated and cultured as described previously (Zhang et al., 2011). The protocol for isolation of neonatal rat cardiomyocytes was approved by the Institutional Animal Care and Use Committee (IACUC) of UT Southwestern and the performance was in adherence to the relevant ethical regulations.

Immunoblotting. Cells were washed once with cold PBS and lysed with the SDS lysis buffer (1% SDS, 10 mM HEPES pH 7.0, 2 mM MgCl₂, 500 U universal nuclease). Protein concentrations were determined by the BCA assay kit (Thermo Fisher Scientific). A total of 20 μg proteins (40 μg proteins for PAR immunoblotting experiments) were loaded onto the SDS-PAGE gel and were then transferred to a nitrocellulose membrane. Nitrocellulose membranes were then blocked with the TBST buffer containing 5% milk (Bio-Rad). Membranes were incubated with the primary antibodies overnight at 4° C. and the secondary antibodies for 1 hour at room temperature (RT). The blots were developed using enhanced chemiluminescence and were exposed on autoradiograph films.

Cycloheximide treatment. HeLa cells (1.5×10⁶) were plated in a 60 mm dish, which were allowed to adhere, and were cultured overnight. Cells were pre-treated with 5 μM iRucaparib or vehicle for 12 hours, then treated with cycloheximide (10 μg/ml). At the indicated time points, cells were washed with cold PBS, and were lysed with the SDS lysis buffer. PARP1 expression was determined by immunoblotting assays.

Real-Time Quantitative PCR. Total RNA was isolated from HeLa cells using the Trizol reagent (Invitrogen) and cDNA was synthesized using the SuperScript III first-strand synthesis kit (Invitrogen). Quantitative PCR reactions were performed on a CFX real-time system using the SYBR Green PCR Supermix according to manufacturer's instructions (ABI). GAPDH levels were used for normalization. Primer sequences (all human genes) are as following: GAPDH forward: 5′-GAGTCAACGGATTTGGTCGT-3′ (SEQ ID NO: 3), GAPDH reverse: 5′-GACAAGCTTCCCGTTCTCAG-3′ (SEQ ID NO: 4); PARP1 forward: 5′-TGGAAAAGTCCCACACTGGTA-3′ (SEQ ID NO: 5), PAPR1 reverse: 5′-AAGCTCAGAGAACCCATCCAC-3′ (SEQ ID NO: 6); PAPR2 forward: 5′-GGCACAAATCAAGGCAGGTTA-3′ (SEQ ID NO: 7), PAPR2 reverse: 5′-AAGTCATGCGGAATCCTGGTG-3′ (SEQ ID NO: 8); PARP3 forward: 5′-GACCAACATCGAGAACAACAACA-3′ (SEQ ID NO: 9), PAPR3 reverse: 5′-GCCTTGTGAAGT GGTTGATCT-3′ (SEQ ID NO: 10).

Quantitative Mass Spectrometry. HeLa cells were left untreated (DMSO) or treated with 5 μM iRucaparib or 5 μM iRucaparib plus 1 μM Rucaparib for 24 hours. For BT-549 cells, cells were left untreated (DMSO) or treated with 5 μM iRucaparib or 20 μM VHL-Rucaparib for 24 hours. Two biological replicate samples were prepared for each treatment condition. Cells were washed with cold PBS and lysed using the SDS lysis buffer. Protein concentrations were determined by the BCA assay (Thermo Fisher Scientific). For each sample, 500 μg of protein was used for the subsequent TMT experiments³⁴. The labeling scheme was as following: HeLa cells: 126 for DMSO-1, 127 for DMSO-2, 128 for iRucaparib-1, 129 for iRucaparib-2, 130 for iRucaparib+Rucaparib-1 and 131 for iRucaparib+Rucaparib-2; BT-549 cells: 126 for DMSO-1, 127 for DMSO-2, 128 for iRucaparib-1, 129 for iRucaparib-2, 130 for VHL-Rucaparib-1 and 131 for VHL-Rucaparib-2.

For the TMT experiments, proteins were reduced with 2 mM DTT for 10 min and alkylated with 50 mM iodoacetamide for 30 min in dark. Proteins were then extracted using methanol-chloroform precipitation. Protein pellets were dissolved in 400 μL 8 M Urea buffer (8 M urea, 50 mM Tris-HCl, 10 mM EDTA, pH 7.5) and were digested by Lys-C (Wako, at a 1:100 enzyme/protein ratio) for 2 hours at RT. The urea concentration was then reduced to 2 M using freshly made 100 mM ammonium bicarbonate solution. Proteins were subsequently digested with trypsin (Thermo Fisher Scientific, at 1:100 enzyme/protein ratio) overnight at RT. Peptides were desalted with Oasis HLB cartridges (Waters) and resuspended in 200 mM HEPES (pH 8.5) to a final concentration of 1 μg/L. For each sample, 100 μg of peptides were reacted with the corresponding amine-based TMT six-plex reagents (Thermo Fisher Scientific) for 1 hour. The reactions were quenched with 5% hydroxylamine solution and were combined.

Samples were desalted and fractioned by bRPLC (basic pH reversed phase HPLC) on a ZORBAX 300 Extend-C18 column (Narrow Bore RR 2.1 mm×100 mm, 3.5 μm particle size, 300 \acute{Å} pore size). Buffer A is (10 mM Ammonium Formate in H₂O, pH 10.0). Gradient was developed at a flow rate of 0.2 mL/min from 0% to 70% buffer B (1 mM Ammonium Formate, pH 10.0, 90% ACN). Seventeen fractions were collected, which were lyophilized, desalted and analyzed by LC-MS/MS as described previously (Hu et al., 2016). Briefly, peptides were separated on a hand-pulled fused silica microcapillary column (75 μm×15 cm, packed with Magic C18AQ, Michrom Bioresources). A 75 min linear gradient was developed ranging from 7% to 32% acetonitrile in 0.1% formic acid at 300 nL/min to elute the peptides (Thermo EASY-nLC system). Samples were then analyzed on an Orbitrap Elite mass spectrometer (Thermo Fisher Scientific) using a top 15 HCD (higher-energy collisional dissociation) method.

MS/MS spectra were searched against a composite database of the human UniProt protein database (02-04-2014) and its reversed complement using the Sequest (Rev28) algorithm. Search parameters allowed for a static modification of 57.02146 Da on cystine (Carbamidomethyl), a variable modification of 15.994915 Da on methionine (oxidation), and a static modification of TMT labels (229.16293 Da) on peptide N-terminus and lysine. For TMT quantification, a 0.03 Th window was scanned around the theoretical m/z of each reporter ion (126:126.127725; 127:127.124760; 128:128.134433; 129:129.131468; 130:130.141141; 131:131.138176). The maximum intensity of each reporter ion was extracted. For each reporter ion channel, the observed signal-to-noise ratio was summed across all quantified proteins and was normalized.

In Vitro PARP1 Activity Assay. The IC₅₀ for Rucaparib and the various CRBN-based PARP1 PROTACs were measured by using a PARP universal chemiluminescent assay kit (Trevigen, #4676-096-K). Briefly, the histone-coated strip wells were hydrated with the assay buffer for 30 min. The various compounds were added and were incubated with the PARP enzyme for 10 min. The reaction cocktail containing biotinylated NAD⁺ was then added into the system, which was incubated for 1 hour to activate PARP1. After the reaction, the wells were washed twice with 0.1% Triton X-100/PBS and twice with PBS. The wells were incubated with Strep-HRP for another hour and were washed again as described above. PeroxyGlow reagents were mixed and added into the strip wells, and the biotin signal on coated histone was immediately measured with chemiluminescence on a Synergy microplate reader (Bio-Tek). The well without PARP1, and the well with PARP1 only was used as the negative (0%) and positive (100%) control, respectively.

Quantitative analysis of the Asp- and Glu-ADP-ribosylated proteome. The D/E-ADP-ribosylation analyses were performed as described previously (Zhang et al., 2013; Zhen et al., 2017). Briefly, SILAC-labeled HeLa cells ([¹²C₆¹⁴N₂]lysine and [¹²C₆¹⁴N₄]arginine, “Light”, or [¹³C₆¹⁵N₂]lysine and [¹³C₆¹⁵N₄]arginine, “Heavy”) were pretreated with 2 μM of the PARG inhibitor³⁶ PDD 00017273 for 1 hour, and then were left untreated (DMSO) or treated with 10 μM Rucaparib or iRucaparib for another hour. The cells were challenged with 2 mM H₂O₂ for 5 min and were harvested in SDS lysis buffer. Protein concentrations were determined by the BCA assay kit. The same amounts of proteins (20 mg) were combined between the light and heavy samples. Lysates were reduced with DTT (3 mM for 20 min at RT), alkylated with iodoacetamide (50 mM for 30 min at RT in dark), and digested overnight with Lys-C and then trypsin (both at a 1:100 enzyme/protein ratio). PARylated peptides were enriched using M-aminophenyl-boronic acid-agarose beads and were eluted using 2M NH₂OH.

Samples were analyzed by LC-MS/MS on an Orbitrap Elite mass spectrometer (Thermo Fisher Scientific) using a top 20 CID (Collision-induced dissociation) method. The isolation window and the minimal signal threshold for MS/MS experiments were set to be 2 Th and 500 counts, respectively. MS/MS spectra were searched against a composite database of the human UniProt database (02-04-2014) and its reversed complement using the Sequest (Rev28) algorithm. Search parameters allowed for a static modification of 57.02146 Da on cysteine, a dynamic modification of addition of 15.0109 Da to aspartic acid and glutamic acid, a variable modification of 15.994915 Da on methionine and a variable modification of 8.01420 Da on lysing and 10.00827 Da on arginine (SILAC label), respectively. Search results were filtered to include <1% matches to the reverse database by the linear discriminator function.

Subcellular Proteins Extraction. Cells (1×10⁶) were left untreated (DMSO) or pretreated with Rucaparib or iRucaparib for 24 hours and were challenged with 0.01% MMS for 2 hours. Subcellular proteins were extracted from the cells using a subcellular fractionation kit (Thermo Fisher Scientific, #78840). Briefly, cells were digested with 0.25% trypsin-EDTA solution, and harvested by centrifuge at 500 g for 5 min and were washed once with cold PBS. The cytosolic proteins were isolated by incubating the cells with the CEB buffer for 10 min at 4° C., and centrifuged at 500 g for 5 min. The membrane proteins were isolated by incubating the pellet from previous step with the MEB buffer for 10 min at 4° C., and centrifuged at 3000 g for 5 min. The soluble nuclear proteins were isolated by incubating the pellet from previous step with the NEB buffer for 30 min at 4° C., and centrifuged at 5000 g for 5 min. The chromatin bound proteins were isolated by incubating the pellet from previous step with the NEB buffer supplemented with nuclease for 15 min at RT and centrifuged at 16000 g for 5 min. Protein concentrations were determined by the BCA assay kit.

Cell Viability Assay. HeLa cells were seeded in 96 well plates with a density of 2,000 cells/well. Sixteen hours later, cells were treated with MMS alone or in combination with 10 μM Rucaparib or iRucaparib for 72 hours. Cell viability was determined using a CellTiter-Glo luminescence kit (Promega). Briefly, cells were balanced for 30 min at RT. One hundred microliters of the CellTiter-Glo reagent was added into each well and the cells were incubated for 10 min. The luminescence was measured using a Synergy microplate reader (Bio-Tek). The ATP level in untreated cells was defined as 100%. The viability of the treated cells was defined as the percentage of the ATP level in treated cells, compared to that in the untreated cells.

Cell Cycle Analysis. Cells (1×10⁵) were left untreated (DMSO) or treated with 10 μM Rucaparib or iRucaparib for 48 hours. Cells were then trypsinized and harvested by centrifuge at 300 g for 5 min, fixed with 70% pre-chilled methanol for 2 hours at −20° C. Fixed cells were washed once with PBS and incubated with RNase A (250 μg/mL) in PBS for another 2 hours at 37° C. and then stained with PI (2 μg/mL). Stained cells were analyzed using BD FACS lyric and analyses were performed using the Flowjo software.

ATP and NAD⁺ measurement. ATP levels were measured by a luminescence assay using the CellTiter-Glo luminescent kit (Promega). Cells were left untreated (DMSO) or pretreated with 5 μM Rucaparib or iRucaparib for 24 hrs, and then were challenged with 0.01% MMS for 9 hours. The cells were balanced for 30 min at RT. One hundred microliters of the CellTiter-Glo reagent was added into each well and incubated for 10 min. ATP levels were determined according the manufacturer's instruction. NAD⁺ levels were assayed using an NAD/NADH quantification kit (Sigma). Cells were pretreated with Rucaparib or iRucaparib for 24 hrs, and then were challenged with 0.01% MMS for 4 hours. Approximately 2×10⁵ cells were washed with 1 mL cold PBS and lysed in 400 μl of the extraction buffer. The total NAD⁺/NADH levels were extracted following two cycles of freezing on dry ice for 20 min followed by 10 min at RT. The samples were vortexed for 15 sec and centrifuged at 13,000 g for 10 min to remove the insoluble materials. NAD⁺ was quantified according to the manufacturer's instruction (Sigma). Fifty microliters of the extracts were used in the NAD⁺ assay, and the values were normalized by the amount of protein in each sample.

Immunofluorescence Microscopy. C2C12 myoblasts or primary rat cardiomyocytes were plated in 35 mm glass bottomed culture dishes (Mattek) and were cultured overnight. The cells were then left untreated or treated with 10 μM Rucaparib or iRucaparib for 48 hours before washing with PBS. Cells were fixed with 4% Paraformaldehyde for 15 min at RT and were washed three times with PBS. The cells were then permeabilized with 0.1% Triton X-100 in PBS for 5 min and were blocked with 1% BSA in PBS for 30 min. Fixed cells were incubated with a γH2AX antibody (1:200, Cell Signaling Technology, #9718) at 4° C. for overnight, washed three times with PBS for 5 min, and incubated with an Alexa Fluor 488 conjugated anti-rabbit antibody (1:500, Thermo Fisher Scientific, A-11008) for one hour at RT. Cells were washed three times with PBS for 5 min and stained with DAPI (1:1000, Thermo, #62248) for 2 min. Cells were washed with PBS and mounted with the FluorSave reagent (Millipore, #345789). Images were collected on a Zeiss LSM 880 Airyscan inverted microscope.

Statistical Analysis. Statistical analyses (t-tests) were performed using GraphPad Prism software (v7). Data were derived from three biological replicate experiments and presented as the mean±SEM. *p<0.05. **p<0.01. ***p<0.001.

### Example 2—Results

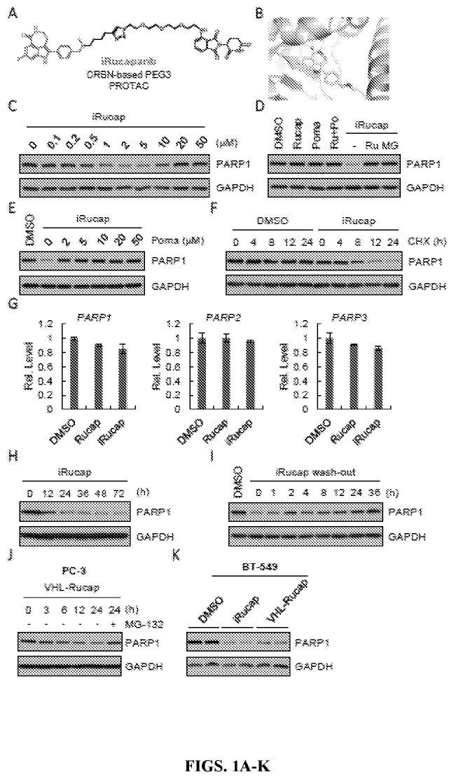

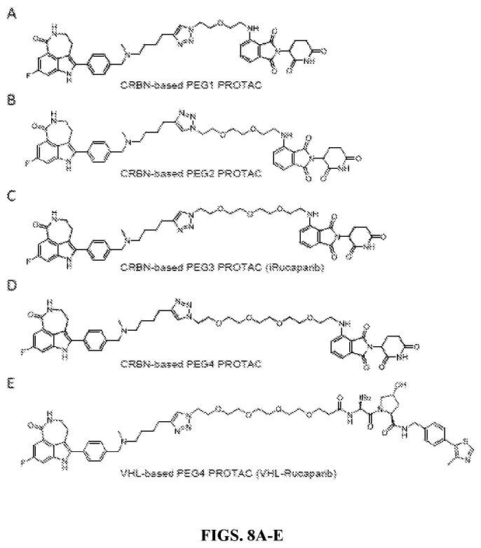

iRucaparib induces proteasome-dependent degradation of PARP1. To pharmacologically induce PARP1 degradation, the inventors designed a series of PROTAC compounds based on an FDA-approved PARP1 inhibitor, Rucaparib (FIG. TA, FIGS. STA-E and supporting information). Rucaparib and the other NAD⁺ competitive PARP1 inhibitors all contain a pharmacophore of an amide (Ferraris, 2010), a moiety that is essential for its inhibitory activity against PARP1 (FIGS. TA-B). Indeed, examination of the co-crystal structure of PARP1 in complex with Rucaparib reveals that the constrained amide in Rucaparib forms a particularly important hydrogen bond network with Ser904 and Gly863 residues within the NAD⁺ binding pocket of PARP1 (Thorsel et al., 2017). The structure also suggests that a tether attached at the amino group distal to the indolyl lactam moiety in Rucaparib would be an ideal position for derivatization (FIG. 1B). Toward this, the inventors functionalized Rucaparib by reductive amination and then used click chemistry to generate bivalent PARP1 PROTACs. For example, pomalidomide (a CRBN ligand) is conjugated through a tri(ethylene glycol) (PEG3) linker to afford Rucaparib-PEG3-pomalidomide (referred to as iRucaparib hereafter) (FIG. TA, FIGS. 8A-E and supporting information).

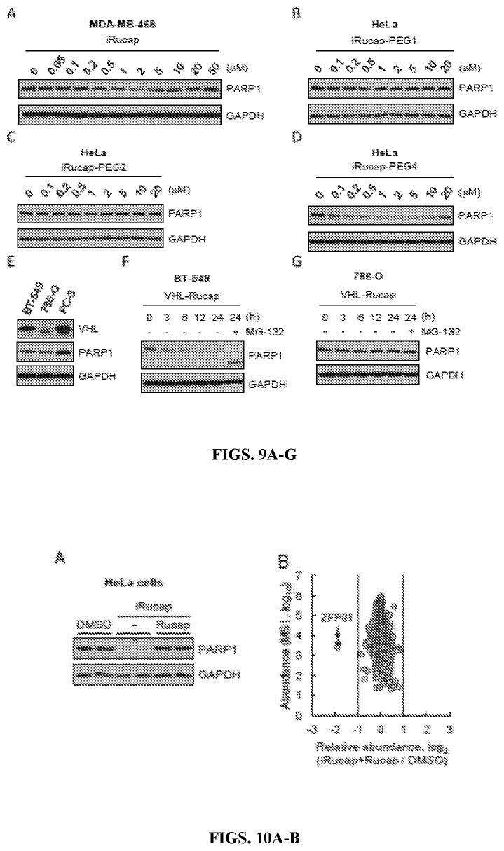

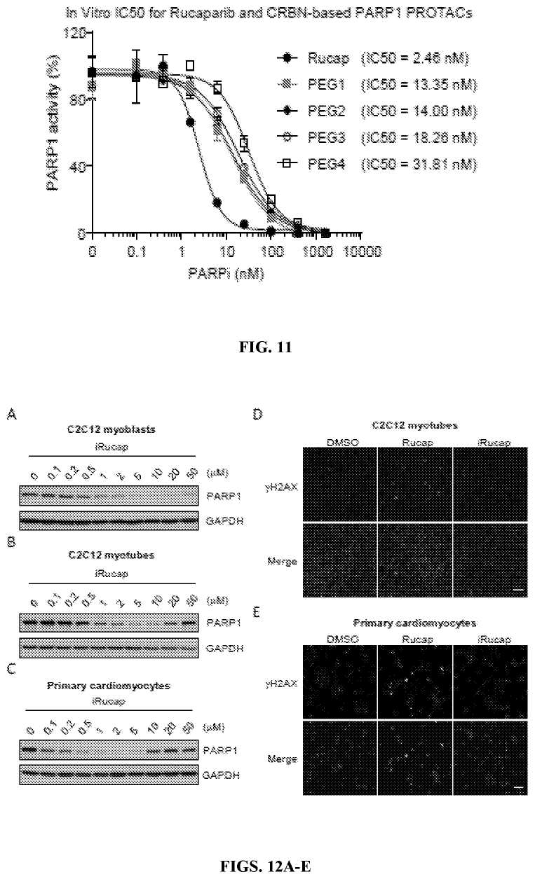

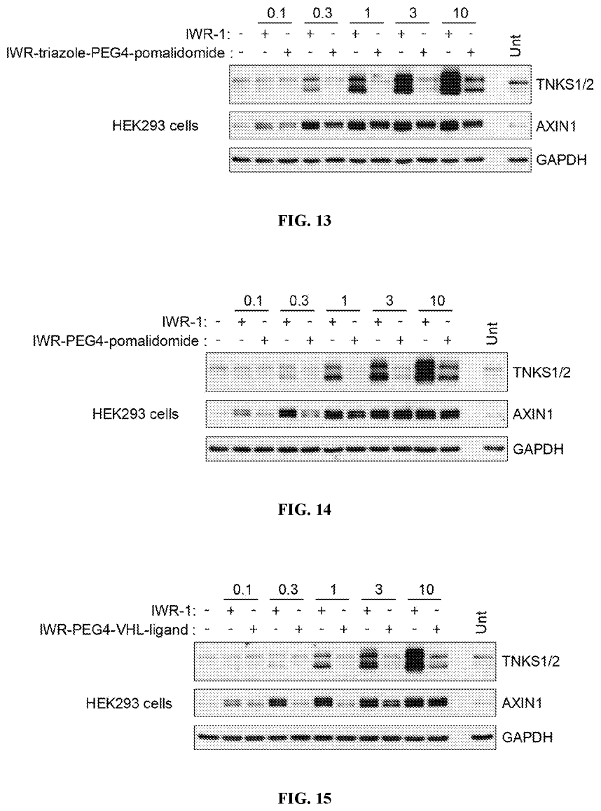

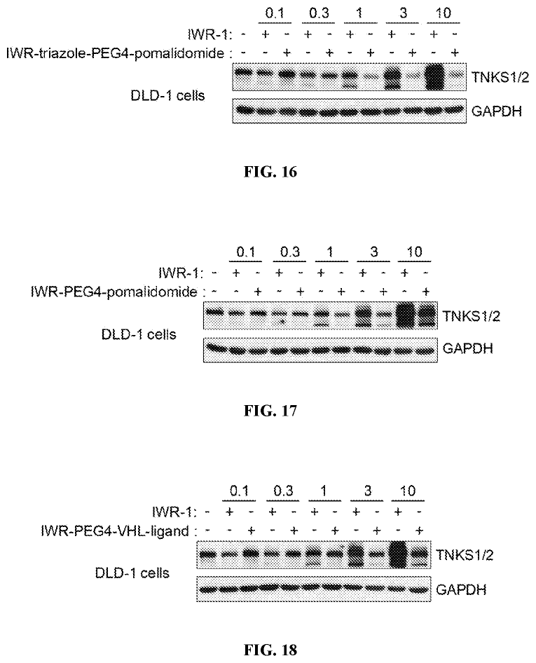

The inventors treated HeLa cells with increasing concentrations of iRucaparib for 24 hours and found that iRucaparib was able to induce robust degradation of PARP1. PARP1 expression was reduced to the lowest level at around 2-5 μM of iRucaparib (FIG. 1C). Notably, at a concentration of 10 μM and above, they they observed a decrease in PARP1 degradation (FIG. 1C). This is consistent with a “hook effect” that has been described previously for other PROTACs (Huang & Dixit, 2016), in which independent engagement of PARP1 and CRBN by iRucaparib attenuates the formation of the PARP1-iRucaparib-CRBN ternary complex, and subsequently, its degradation. iRucaparib was also able to induce PARP1 degradation in MDA-MB-468 cells with an effective concentration of around 2 μM (FIG. 9A).

To determine whether iRucaparib-induced PARP1 degradation is dependent on the ubiquitin-proteasome pathway, the inventors pretreated HeLa cells with the proteasome inhibitor MG132 and found that PARP1 degradation was completely blocked (FIG. 1D). To examine the specificity of iRucaparib, they pretreated the cells with the PARP1 inhibitor Rucaparib, and then treated the cells with iRucaparib. The inventors found that Rucaparib was able to compete with iRucaparib for PARP1 binding, and in doing so, block PARP1 degradation (FIG. 1D). Similarly, pomalidomide treatment was able to prevent iRucaparib from binding to CRBN, and thus block PARP1 degradation (FIG. 1E). In contrast, treating the cells with Rucaparib, pomalidomide or a combination of Rucaparib and pomalidomide had no effect on PARP1 protein levels (FIG. 1D). These results are again consistent with the model in which the engagement of both PARP1 and CRBN in the same complex is required for effective PARP1 degradation.

The inventors treated HeLa cells with cycloheximide (CHX, a eukaryote protein synthesis inhibitor) in combination with either DMSO or iRucaparib. PARP1 levels were not changed in the control cells even after 24 hrs of CHX treatment, which is consistent with the long half-life of this protein (>60 hrs) (Schwanhausser et al., 2011). In contrast, they found that PARP1 had a much shorter half-life in iRucaparib-treated cells, with noticeable protein degradation starting at 8 hrs of CHX treatment (FIG. 1F). These results suggest that iRucaparib post-translationally regulates the stability of PARP1. To further corroborate this, the inventors performed real time quantitative PCR analyses and found that PARP1 mRNA levels were not significantly altered upon the treatment of either Rucaparib or iRucaparib (FIG. 1G). Similarly, the mRNA levels of PARP2 and PARP3 were also unchanged by Rucaparib or iRucaparib treatment.

To investigate the kinetics of iRucaparib-induced PARP1 degradation, the inventors performed a time course experiment where they treated HeLa cells with iRucaparib and found that nearly 50% PARP1 was degraded after 12 hours treatment. PARP1 abundances further decreased and persisted for at least 72 hrs (FIG. 1H). These data indicate that, although PARP1 is a highly abundant and stable protein, the CRBN-based PROTAC system is able to efficiently degrade this protein. The inventors also performed washout experiments to examine whether the effect of iRucaparib is reversible, and to determine the duration of iRucaparib-induced PARP1 degradation. They observed that PARP1 levels were partially recovered after 4-8 hrs of compound washout but required about 36 hrs to reach the pre-treatment level (FIG. 1I).

Recent structural studies showed that linker lengths could have a profound impact on the efficacy and selectivity profiles of the PROTAC compounds (Gadd et al., 2017; Cyrus et al., 2011). Indeed, the inventors found that PROTACs with a PEG1 or PEG2 linker (FIGS. 8A-E) was not able to degrade PARP1. In contrast, the PEG4 PROTAC was able to degrade PARP1 as efficiently as iRucaparib (i.e., the PEG3 PROTAC) (FIGS. 9B-D). These results indicate that longer PEG linkers allow the optimal formation of the ternary complex that is competent for PARP1 degradation.

In addition to the abovementioned CRBN-based PROTACs, the inventors also synthesized another PROTAC compound (VHL-Rucaparib) wherein a VHL ligand (the HIF1a-derived-hydroxyproline compound that binds to the E3 ligase VHL (Galdeano et al., 2014) is conjugated to Rucaparib via a PEG4 linker (FIGS. 8A-E). To examine the PARP1 degradation by VHL-Rucaparib, they treated two VHL-proficient cells (BT549 and PC-3) and one VHL-deficient cell (786-O) with VHL-Rucaparib for 24 hours and examined PARP1 levels (FIG. 9E). The results showed that PARP1 was successfully downregulated by VHL-Rucaparib in both VHL-proficient cells (i.e., BT-549 and PC-3) (FIGS. 1J-K), but not the VHL-deficient cells (i.e., 786-O) (FIG. 9G). Furthermore, VHL-Rucaparib-induced PARP1 degradation in PC-3 cells could be rescued by MG-132 treatment, suggesting that VHL-based PROTACs also require the ubiquitin-proteasome system for target degradation (FIG. 1J). Notably, combination of iRucaparib and MG-132 treatment caused apoptosis, and as a result, extensive PARP1 cleavage in BT-549 cells (FIG. 9F). Finally, the inventors found that both VHL-Rucaparib and iRucaparib were able to achieve robust PARP1 degradation in BT549 cells (FIG. 1K).

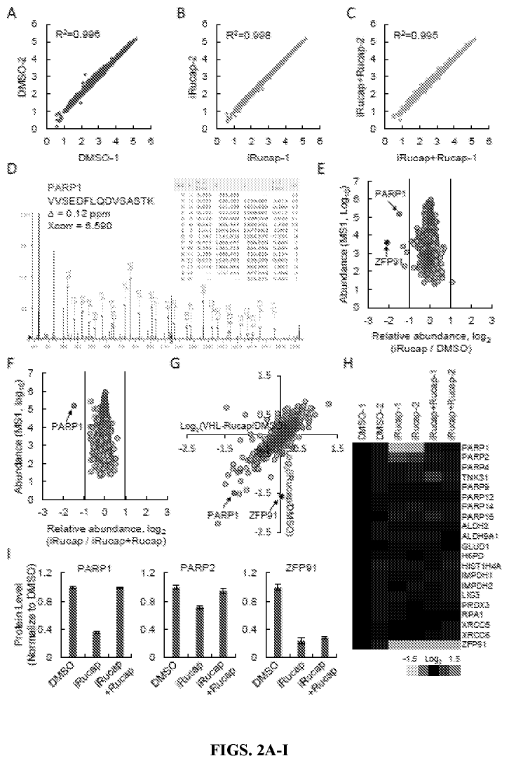

iRucaparib selectively targets PARP1 for degradation. To characterize the effects of iRucaparib in an unbiased manner, the inventors performed global protein expression analysis using a TMT (tandem mass tag)-based, multiplexed quantitative mass spectrometric approach. Proteome-wide expression profiling was performed on HeLa cells where they included two independent biological replicate samples for the following three conditions: (1) DMSO treatment; (2) iRucaparib treatment; and (3) iRucaparib plus Rucaparib treatment (FIGS. 2A-I and FIG. 10A). After lysis, proteins were reduced, alkylated and digested, with the resulting peptides labeled by the TMT reagents. The pooled TMT-labeled peptides were subject to two-dimensional HPLC separation and were analyzed by high sensitivity MS and MS/MS experiments (Hu et al., 2016). From the above-mentioned 6-plex sample, the inventors were able to identify and quantify a total of 202,802 peptides from 7,437 proteins (peptide false discovery rate=0.16% and protein false discovery rate=1%). A representative MS2 spectrum leading to the identification and quantification of a PARP1 peptide (VVSEDFLQDVSASTK; SEQ ID NO: 1) is shown in FIG. 2D. Correlation analysis revealed that an excellent reproducibility was achieved in the inventors' quantification analysis between the biological replicate analyses (i.e., R²=0.996 for the DMSO treatment group; R2=0.998 for the iRucaparib treatment group; and R2=0.995 for the iRucaparib plus Rucaparib treatment group) (FIGS. 2A-C).

The inventors first extracted the protein expression data of the DMSO and iRucaparib group and performed a binary comparison. iRucaparib caused rather limited perturbation of the overall proteome, with the abundance of only five proteins decreased by more than 2-fold after the treatment. A protein called ZFP91 ranked as the most downregulated protein among the 7,437 quantified proteins, with a Log 2 ratio of −2.09 (Log 2(iRucaparib/DMSO)). Using a SILAC-based quantitative proteomic approach, a recent study reported ZFP91 as a lenalidomide/pomalidomide (IMiD)-dependent substrate of the ubiquitin E3 ligase CRBN (An et al., 2017). It was shown that ZFP91 harbors a zinc finger (ZnF) motif that mediates a high-affinity interaction with the CRBN-IMiD complex. In this case, the inventors reasoned that the depletion of ZFP91 most likely results from the activity of the IMiD moiety (pomalidomide) within iRucaparib. The identification of a known IMiD-dependent substrate of CRBN validates the sensitivity and sequencing depth of the inventors' quantitative proteomic platform.

Intriguingly, among the quantified proteins, PARP1 ranked as the second most downregulated protein, with a Log 2(iRucaparib/DMSO) ratio of −1.49 (FIGS. 2E, 2H and 2I). In this dataset, the inventors were also able to identify a number of other PARPs, including PARP2, PARP16, PARP14, PARP4, PARP12, PARP9 and TNKS1 (FIG. 2H). Even though the parent compound Rucaparib binds to several of these PARPs (PARP2, PARP16, PARP4 and TNKS1) (Wahlberg et al., 2012), only PARP2 showed about 30% decrease after iRucaparib treatment (FIGS. 2H-I), and none of the other PARPs exhibited significant downregulation in response to this compound (FIG. 2H). In addition to various PARPs, a recent chemoproteomic study demonstrated that Rucaparib is also able to interact with other NAD⁺/NADP⁺-utilizing enzymes, including hexose-6-phosphate dehydrogenase (H6PD) and aldehyde dehydrogenase, mitochondria (ALDH2) (Knezevic et al., 2016). The inventors found that these two proteins also showed no downregulation upon the treatment of iRucaparib (FIG. 2H), with a Log 2(iRucaparib/DMSO) ratio of 0.02 and 0.05, respectively, indicating that they are not targeted by iRucaparib for CRBN-dependent degradation.

Next, the inventors extracted the protein expression data of the iRucaparib vs. iRucaparib/Rucaparib group. As shown in FIG. 1D and FIG. 10A, Rucaparib was able to compete with iRucaparib for PARP1 binding and therefore block iRucaparib-induced PARP1 degradation. The inventors reasoned that this comparison would allow the identification of protein degradation events that are specifically mediated by the Rucaparib moiety within iRucaparib. Intriguingly, this analysis led to the identification of PARP1 as the only protein that showed a decrease of more than 2-fold among the 7,437 quantified proteins (FIG. 1F). Finally, they conducted a comparison between the DMSO vs. iRucaparib/Rucaparib group, which allows the dissection of potential IMiD-dependent substrates of CRBN. Again, using a 2-fold decrease as the threshold, the inventors identified two proteins UBBP4 and ZFP91 as the potential pomalidomide-induced protein degradation targets (FIG. 10B). In summary, these results show that although Rucaparib itself is a quite promiscuous PARP inhibitor, the PROTAC approach represents an exceptionally specific strategy for PARP1 degradation.

Finally, using a similar TMT approach, the inventors characterized the proteome changes in BT-549 cells treated with either iRucaparib or VHL-Rucaparib (FIG. 2G). In this case, they were able to quantify a total of 8,207 proteins (protein false discovery rate=1%). Again, the inventors identified ZFP91 as an iRucaparib target protein, with a decrease of ˜3-fold after the compound treatment. Intriguingly, the abundance of this protein did not show any changes after VHL-Rucaparib treatment, suggesting that it is not targeted by this VHL-PROTAC for degradation. In both iRucaparib and VHL-Rucaparib groups, PAPR1 represented as one of the most significantly downregulated proteins (FIG. 2G). These results indicate that both compounds are able to achieve highly efficient and specific degradation of PARP1.

The effects of iRucaparib on ADP-ribosylation-dependent signaling. To study how the PARP1 downstream signaling network is affected by the treatment of PARP1 PROTACs (FIGS. 3A-D), the inventors first examined the biochemical activity of the various CRBN-based PARP1 PROTACs in vitro (FIG. 11). They found that the IC₅₀ for the parent compound Rucaparib is 2.46 nM, which is similar to that reported in a recent study (Knezevic et al., 2016). The CRBN-series PARP1 PROTACs maintained nanomolar biochemical inhibition of PARP1 (e.g., iRucaparib, PEG3, IC₅₀=18.26 nM). The slight decrease in the inhibitory activity of iRucaparib is not surprising, due to the potential steric hindrance introduced by the linker creating unfavorable interactions with the helical subdomain (HD) of PARP1 (FIG. 1B).

Next, the inventors examined the inhibitory activity of iRucaparib in intact cells. HeLa cells were pretreated with a cell-permeable PARG inhibitor, PDD00017273 for 1 hr to allow the accumulation of PARylated proteins. Cells were then treated with Rucaparib or iRucaparib for 1 hr and challenged with H₂O₂. Because PARP1 can also be activated by sheared DNA, the inventors harvested cells using an SDS buffer to inactivate PAR-metabolizing enzymes, and to prevent artificial introduction/removal of PARylation during cell lysis (Zhen & Yu, 2018). The results showed that PARP1 was completely blocked by acute treatment (1 hr) of both Rucaparib and iRucaparib, as shown by the loss of the PAR signal as well as the reversal of PARP1 mobility shift (FIG. 3B).

To characterize how individual PARylation events respond to Rucaparib vs. iRucaparib, the inventors performed global analysis of the Asp/Glu-ADP-ribosylated proteome that is regulated by these two compounds (FIG. 3A). Specifically, they generated SILAC (stable isotope labeling by amino acids in cell culture)-labeled HeLa cells and treated the light and heavy cells with PDD00017273 (1 hr). The light and heavy cells were then treated with DMSO and Rucaparib (1 hr), respectively. Both cells were then treated with H₂O₂ and the lysates were combined at a 1:1 ratio. PARylated peptides were enriched with boronate affinity chromatography, and were eluted with NH₂OH (Zhang et al., 2013; Zhen et al., 2017). In a second experiment, the inventors also performed a similar analysis using iRucaparib (1 hr treatment) (FIG. 3A). They were able to identify and quantify a total of 399 and 424 PARylated peptides from the Rucaparib and iRucaparib group, respectively. Using the same NH₂OH chemistry, the inventors previously characterized the Asp-/Glu-ADP-ribosylated proteome in a panel of breast cancer cell lines (Zhen et al., 2017). Comparison with the current data identified a number of common PARylated proteins, including PARP1, FUS, HNRNPU, DDX21, THOC4, PCNA, CHTOP, TAF15, among others. These results indicate that upon sensing DNA damage, PARP1 is able to modify a core set of proteins that underlie its basic function (e.g., mediating DNA damage response) independent of the specific cellular contexts.

As expected, the inventors observed a dramatic change in the overall Asp-/Glu-ADP-ribosylated proteome after Rucaparib treatment, with many Asp/Glu-ADP-ribosylation sites showing exquisite sensitivity to this compound. For example, the abundance of a PARP1 auto-modified peptide AEPVE*VVAPR (SEQ ID NO: 11; * indicates the site of ADP-ribosylation) decreased by more than 99% after Rucaparib treatment (FIG. 3C). As a control, a non PARylated peptide (HQSFVLVGETGSGK; SEQ ID NO: 2) from DHX15 showed a 1:1 ratio between the light and heavy cells. The PARP1 automodified peptide AEPVE*VVAPR (SEQ ID NO: 11) was also detected in the iRucaparib experiment, which showed a very similar response to iRucaparib (FIG. 3C). The inventors then extracted the SILAC ratio for all the PARylated peptides identified in the Rucaparib vs. iRucaparib experiments and found that the change in protein ADP-ribosylation correlated well between the two compounds (FIG. 3D). These results indicate that despite the addition of the PROTAC moiety, iRucaparib retains the capability to potently inhibit the catalytic activity of PARP1 and its downstream signaling in intact cells.

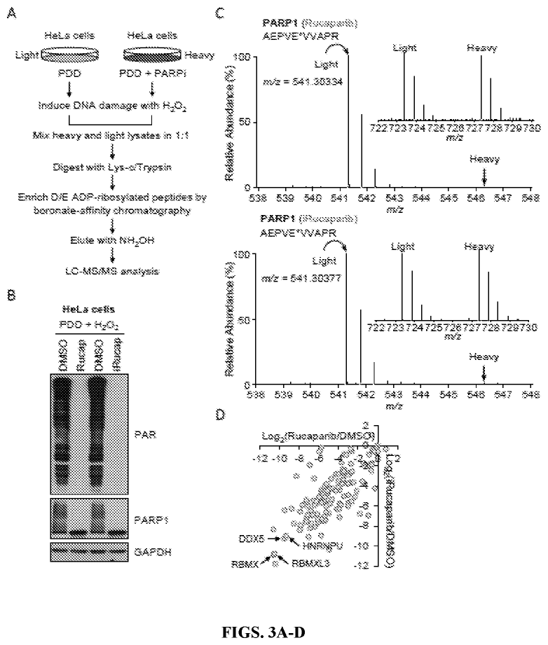

Decoupling of PARP1 catalytic inhibition from PARP1 trapping by iRucaparib. Unlike regular PARP1 inhibitors that simultaneously cause PARP1 inhibition and trapping, the inventors reason that the treatment of PARP1 PROTACs leads to PARP1 degradation, a property that can be used to dissect these two pathways (i.e., catalytic inhibition vs. trapping). Towards this, the inventors first treated HeLa cells with 5 μM Rucaparib or iRucaparib. The cells were then challenged with MMS (an agent that induces alkylating DNA damage), and subject to subcellular fractionation. Under the control conditions (without the treatment of MMS), most PARP1 was in the nuclear soluble fraction. They found that MMS treatment alone led to a slight enrichment of PARP1 in the chromatin-bound fraction (FIG. 4A). Consistent with the previous studies, PARP1 trapping on chromatin was dramatically increased after the cells were treated with a combination of MMS and Rucaparib. In contrast, PARP1 was degraded upon the treatment of iRucaparib, and was undetectable in the chromatin-bound fraction from the cells treated with MMS and iRucaparib (FIG. 4A). The inventors also compared a number of PARP1 inhibitors, and in keeping with recent studies (Murai et al., 2012; 2014), their potency in PARP1 trapping varied with BMN673>Niraparib>Olaparib>Rucaparib>Veliparib (FIG. 4B). iRucaparib induced an even lower amount of trapped PARP1 compared to the least potent PARP1-trapper, Veliparib, with a level reaching that observed in cells without the treatment of the DNA-damaging agent (MMS) (FIG. 4B).

The trapped PARP1/DNA protein complex is known to impair replication fork progression and subsequently, induce a DNA damage response (Murai et al., 2012). To determine the contribution of trapped PARP1 in mediating the cytotoxicity of PARP1 inhibitors under the basal conditions, the inventors treated HeLa cells with DMSO, Rucaparib or iRucaparib for 72 hrs. Cell cycle analyses revealed that Rucaparib, but not iRucaparib, induced G2 accumulation (FIGS. 4C-D). In addition, treatment of Rucaparib but not iRucaparib caused DNA damage (as shown by the accumulation of γH2AX) and suppressed the proliferation of these cells (FIGS. 4E-F). These data are consistent with a model where spontaneously generated base lesions are recognized by PARP1, which, upon Rucaparib treatment, leads to the formation of trapped PARP1 and causes cell death. These toxic PARP1/DNA complexes, however, are abrogated by PARP1 PROTACs, resulting reduced suppression of cell survival and proliferation. Finally, the inventors treated HeLa cells with either MMS, or MMS in combination with Rucaparib or iRucaparib. They found that although MMS alone slightly decreased cell survival, the degree of cell death was dramatically enhanced when cells were treated with a combination of MMS and Rucaparib. On the contrary, iRucaparib in combination with MMS only resulted in a moderate increase in cell death compared to MMS alone (FIG. 4G). These results indicate that PARP1 trapping is a critical mediator of the cytotoxic effects of PARP1 inhibitors under both basal and genotoxic conditions.

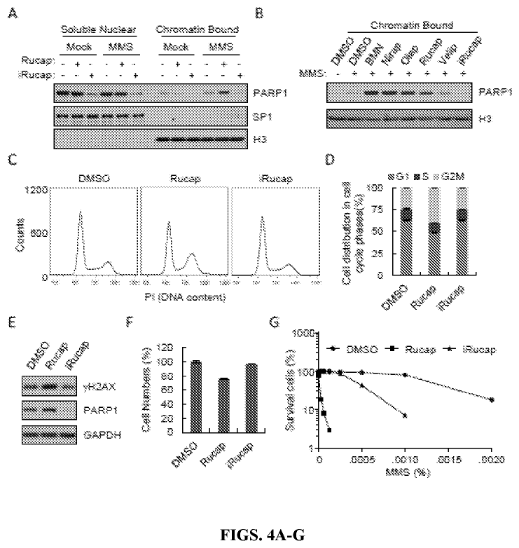

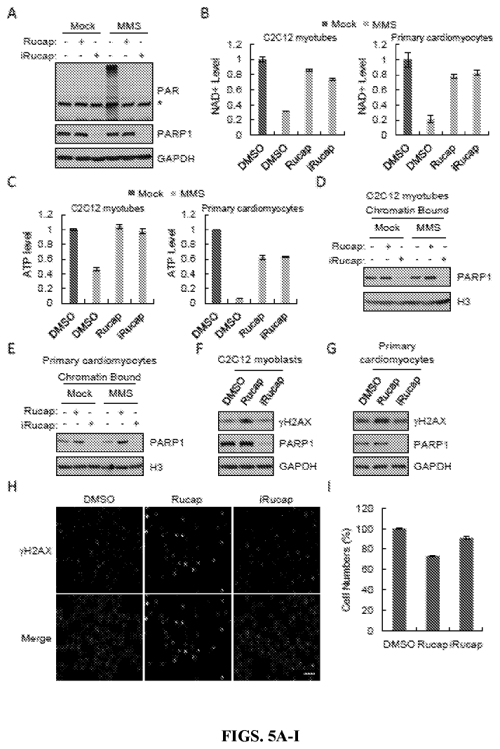

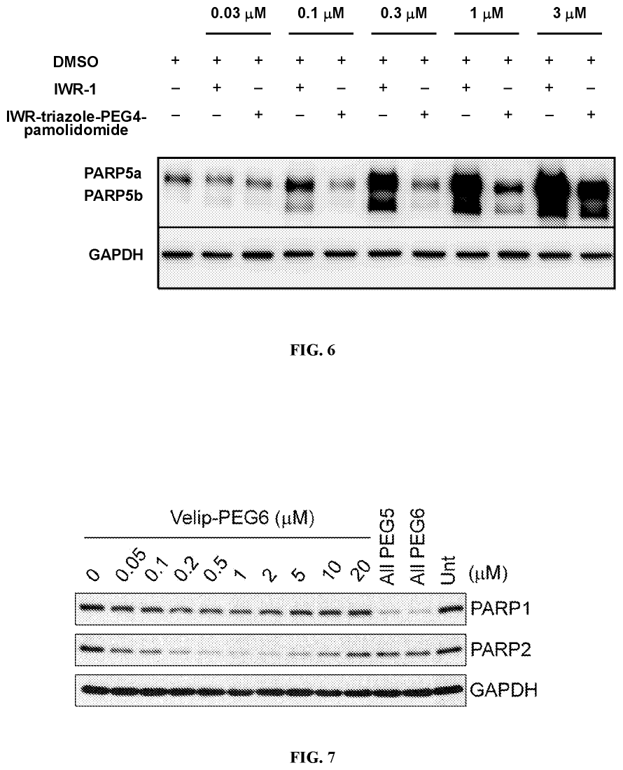

iRucaparib protects muscle cells and primary cardiomyocytes against genotoxic stress-induced cell death. PARP1 is hyperactivated upon sensing oxidative DNA damage during IR injury. This results in NAD⁺/ATP depletion, energy crisis, and eventually PARP1-dependent cell death. However, because of the significant cytotoxicity associated with PARP1 trapping, the current PARP1 inhibitors are less useful in these circumstances. The inventors sought to test whether iRucaparib treatment mimics PARP1 genetic deletion, and therefore protects cells against genotoxic stress-induced cell death. First, they found that iRucaparib treatment resulted in efficient PARP1 degradation in mouse C2C12 myoblasts, and fully differentiated C2C12 myotubes, with maximum degradation occurring at around 5˜10 μM (FIGS. 12A-B). In addition, they also observed robust iRucaparib-induced PARP1 degradation in primary rat cardiomyocytes (FIG. 12C). Importantly, MMS-induced PARP1 activation was completely blocked by pretreating C2C12 myotubes with either Rucaparib or iRucaparib (FIG. 5A). Next, the inventors examined PARP1 trapping in C2C12 myotubes, and the results showed that the amount of PARP1-DNA complexes was dramatically increased upon the treatment of Rucaparib and MMS (FIG. 5D). However, upon the treatment of iRucaparib, PARP1 became undetectable in the chromatin-bound fraction, in both the control and the MMS treatment group. Similar results were also obtained in primary cardiomyocytes (FIG. 5E). Accordingly, Rucaparib, but not iRucaparib treatment resulted in robust accumulation of γH2X in C2C12 cells (FIG. 5F, FIG. 5H and FIG. 12D) and primary cardiomyocytes (FIG. 5G and FIG. 12E).

The inventors then examined whether iRucaparib protects cells against genotoxic stress-induced cell death. First, they studied how the bioenergetic status of a cell is modulated following DNA damage with or without the treatment of PARP1 inhibitors. They pretreated C2C12 myotubes with Rucaparib or iRucaparib, challenged the cells with MMS, and measured cellular NAD⁺ levels (FIG. 5B). Consistent with PARP1 activation being a major mechanism for NAD⁺ catabolism, DNA alkylation damage caused a dramatic decrease in the NAD⁺ levels in these cells (31% of that in control cells). In contrast, NAD⁺ decrease was potently blocked in cells that were pre-treated with either Rucaparib (86% of that in control cells) or iRucaparib (74% of that in control cells) (FIG. 5B). Similar results were also obtained in primary cardiomyocytes (FIG. 5B). Depletion of the cellular NAD⁺ pool leads to forced NAD⁺ synthesis through the salvage pathway, which dramatically lowers the cellular ATP level. Indeed, the inventors found that ATP abundances in C2C12 cells and primary cardiomyocytes were dramatically lowered upon MMS treatment, which could be rescued by treating the cells with either Rucaparib or iRucaparib (FIG. 5C). Finally, consistent with their respective roles in mediating PARP1 trapping, they found that Rucaparib, but not iRucaparib caused a marked decrease in the proliferation of C2C12 myoblasts (FIG. 5I).

### Example 3—Discussion

PROTAC has emerged as a promising technology for the targeted degradation of a protein of interest²⁶. Unlike the traditional catalytic inhibitors, PROTACs simultaneously induce pharmacological inhibition and depletion of the target protein. In doing so, PROTACs abrogates the potential scaffolding function of this protein, a property that is uniquely suitable for the development of chemical probes that dissect PARP1 inhibition vs. trapping mechanisms. Here, the inventors designed and characterized a series of PARP1 PROTACs using both CRBN and VHL-based degradation systems. Among these compounds, iRucaparib, a bivalent molecule consisting of Rucaparib conjugated to pomalidomide via a PEG3 linker emerged as a promising candidate compound that achieves robust PARP1 degradation.

PROTACs have been successfully deployed to induce the degradation of a number of signaling proteins. Compared to these studies, higher concentrations of PAPR1 PROTACs were used in this study to achieve efficient PARP1 degradation. However, unlike these signaling molecules, PARP1 is a highly abundant protein (approximately 1-2 million molecules per cell) (Yamanaka et al., 1988) with a long half-life (>60 hrs) (Schwanhausser et al., 2011). In addition, the efficiency of PROTAC-mediated protein knockdown is also affected by several other factors, including (1) the expression level of the relevant E3 ligase in a cell (i.e., CRBN for iRucaparib, and VHL for VHL-Rucaparib) (FIG. 9E). (2) the efficiency of ubiquitin transfer between the E3 ligase and the target protein; (3) the rate at which the ubiquitinated protein is transported and processed by the proteasome; and (4) the rate of de-ubiquitination and de novo protein synthesis. Future studies are warranted to examine how each of these factors contributes to the efficiency of a PARP1 PROTAC compound in a specific cellular context.

A number of recent structural and biochemical studies have shown that in order to induce efficient target degradation, bivalent PROTAC compounds have to recruit their respective targets into productive proximity. This is a highly dynamic process, which also involves “back-folding” of the linker to form additional linker-protein contacts that are critical for the affinity observed for various PROTACs (Gadd et al., 2017). Indeed, depending on the nature of the E3 ligase system, linker lengths could have a profound impact on the efficacy of these PROTAC compounds (Cyrus et al., 2011). The inventors therefore evaluated a number of CRBN-based PARP1 PROTACs with different linker lengths (i.e., PEG1, PEG2, PEG3 and PEG4, respectively). Although compounds with a PEG1 or PEG2 linker maintained catalytic inhibitory activity against PARP1 (FIG. 11), they were not able to induce PARP1 degradation. In contrast, the PEG4 PROTAC was able to degrade PARP1 as efficiently as iRucaparib (i.e., the PEG3 PROTAC) (FIG. 9D). Similarly, PARP1 was also robustly degraded by a PEG4 VHL PROTAC compound in several cell lines. These results suggest that the formation of a PARP1/PROTAC/E3 ternary complex with optimal linker lengths is critical for the ubiquitination and subsequent degradation of PARP1.

All of the current PARP1 inhibitors block this enzyme by competitively occupying the NAD⁺-binding pocket, a domain that is highly conserved among all PARPs. As a result, it is likely that these PARP1 inhibitors will have additional targets, including other PARPs and/or NAD⁺-binding proteins (similar to many promiscuous ATP-competitive kinase inhibitors). Indeed, Wahlberg et al. performed a systematic evaluation of the off-target effects of PARP1 inhibitors by profiling the interactions between 13 recombinant PARP catalytic domains with a library of 185 such compounds (Wahlberg et al., 2012). They found that in addition to its primary target (PARP1), Rucaparib also binds to many other PARP proteins, including PARP2, PARP3, PARP4, TNKS1, TNKS2, PARP10, PARP15 and PARP16.

Furthermore, Knezevic et al. used a chemoproteomic approach and showed that Rucaparib also interacts with other NAD⁺/NADP⁺-utilizing enzymes, including H6PD and ALDH2 (Knezevic et al., 2016). Similar to degradation efficiency, the specificity of PROTAC compounds could also be affected by the length and composition of the linker. Although a PROTAC compound might bind to multiple targets, only a subset of these proteins might be brought into proximity in a manner that allows favorable protein-protein interaction (i.e., without opposing charged surfaces), and efficient ubiquitination (i.e., with accessible Lys residues for ubiquitin transfer). In this regard, the selectivity of a promiscuous binding ligand might be improved by an optimized the linker chemistry, when this compound is incorporated into a PROTAC molecule. Using an isobaric labeling based quantitative proteomic strategy, the inventors quantified more than 7,400 proteins, and showed that PARP1 was one of the most downregulated proteins in the iRucaparib-treated samples. Besides PARP1, they were also able to detect a number of other Rucaparib-binding proteins, including PARP2, PARP4, TNKS1, PARP16, H6PD and ALDH2, etc. Intriguingly, among these proteins, only PARP2 showed about 30% decrease after iRucaparib treatment, and none of the other Rucaparib target proteins exhibited significant downregulation in response to iRucaparib treatment. These results indicate that iRucaparib represents an excellent PROTAC compound to achieve highly efficient and specific degradation of PARP1.

It has been proposed that PARP1 inhibitors kill tumors via two distinct but interconnected mechanisms. First, by catalytically inhibiting PARP1, these compounds block PARylation and PARylation-dependent DDR. Second, PARPi may also kill tumor cells by inducing PARP1 trapping. In this case, it has been proposed that the binding of PARPi to the NAD⁺ site allosterically enhances the affinity of PARP1 to DNA lesions, resulting in the formation of toxic PARPi-PARP1-DNA complexes (Murai et al., 2012). Although several PARP1 inhibitors have similar catalytic inhibition potency, they demonstrate markedly different cytotoxicity, an effect that could be ascribed to their unequal PARP1 trapping abilities (Leutert et al., 2018). The inventors reasoned that by degrading PARP1, PARP1 PROTACs offer a pharmacological approach to dissecting PARP1 inhibition vs. trapping in mediating the cytotoxic effects. They first showed that despite the presence of the bulky linker and the pomalidomide moiety in the molecule, iRucaparib retains the capability to potently inhibit the catalytic activity of PARP1 both in vitro and in intact cells. Using boronate affinity enrichment combined with NH₂OH derivatization, the inventors then showed that both Rucaparib and iRucaparib were able to block the ADP-ribosylation of PARP1 substrates, indicating that the downstream, PARylation-mediated signaling network of PARP1 responded similarly to the treatment of these two compounds. Next, the inventors showed that Rucaparib greatly induced PARP1 accumulation in the chromatin-bound fraction, when cells were co-treated with a DNA-damaging agent. In contrast, under these conditions, PARP1 was degraded by iRucaparib treatment, and hence was not detected in the chromatin-bound fraction. Consistent with these findings, iRucaparib treatment did not induce DDR, and had no effect on cell cycle progression or cell proliferation. Finally, they demonstrated that co-treatment of cells with MMS and Rucaparib led to massive cell death. On the other hand, the cytotoxicity of MMS and iRucaparib co-treatment was much weaker. These results are consistent with a model in which PARP1 trapping is likely to be a main contributor of the anticancer activity of PARP1 inhibitors.

Besides the role in mediating cell stress responses in cancer cells, PARP1 is hyperactivated upon sensing DNA damage caused by other pathophysiologically relevant triggers, including those produced during ischemia-reperfusion (IR) in cardiac myocytes, NMDA receptor activation in neurons, endotoxin stimulation in macrophages, and elevated extracellular glucose in endothelial cells (Pedrioli et al., 2018). In these circumstances, DNA damage-induced PARP1 activation is known to cause the collapse of the bioenergetic cycle, and eventually cell death (e.g., necrosis). PARP1 is also known to regulate another form of programmed cell death called parthanatos⁵⁰. During parthanatos, the accumulation of PAR causes the translocation of apoptosis-inducing factor (AIF) from mitochondria to the nucleus (Yu et al., 2002; Wang et al., 2009). Recently studies have shown that AIF is able to bind to a nuclease called macrophage migration inhibitory factor (MIF). In so doing, AIF recruits MIF to the nucleus, which cleaves genomic DNA, and eventually leads to parthanatos (Wang et al., 2016).

Genetic deletion of PARP1 in animals has been shown to provide profound protection against a number of diseases, including IR injury (e.g., stroke, cardiac infarction and ischemia renal injury) (Eliasson et al., 1997; Yang et al., 2000; Zheng et al., 2005), cerebellar ataxia (Hoch et al., 2017), streptozocin-induced diabetes (Li et al., 2013) and neurodegeneration (Kam et al., 2018). Thus, it has been proposed that PARP1 inhibitors, by preventing PAR formation and NAD⁺ depletion, have the potential to maintain cellular bioenergetics and sustain cell viability, under these genotoxic stress conditions. The inventors showed that iRucaparib treatment mimics PARP1 genetic deletion in muscle cells and primary cardiomyocytes. Specifically, iRucaparib was able to inhibit the catalytic activity of PARP1 and protect these cells from genotoxic stress-induced NAD⁺/ATP depletion. However, unlike Rucaparib, iRucaparib treatment does not cause PARP1 trapping, DNA damage or growth suppression.

In summary, the inventors have designed and evaluated a series of PARP1 PROTAC compounds based on an FDA-approved PARP1 inhibitor, Rucaparib. By fine-tuning the lengths of the linker, as well as the specific E3 ligase system, they identified iRucaparib as a promising PARP1 PROTAC molecule that achieves highly potent and specific degradation of PARP1. Using this compound, they were able to dissect the contribution of PARP1 catalytic inhibition vs. PARP1 trapping in mediating the cytotoxicity of PARP1 inhibitors in cancer cells. Treatment of this compound in non-cancerous cells mimics PARP1 genetic deletion, which protects cells against DNA damage-induced PARP1 activation and energy depletion, without eliciting the deleterious PARP1 trapping. Besides offering mechanistic insights into PARP1 trapping, these results suggest that PARP1 PROTACs represent a highly promising strategy for the treatment of pathological conditions caused by PARP1 hyperactivation.

Future studies are warranted to fully define the therapeutic potential of this class of compounds.

### Example 4—Synthetic Schemes

### Rucaparib-Triazole-PEG1-Pomalidomide (1a)

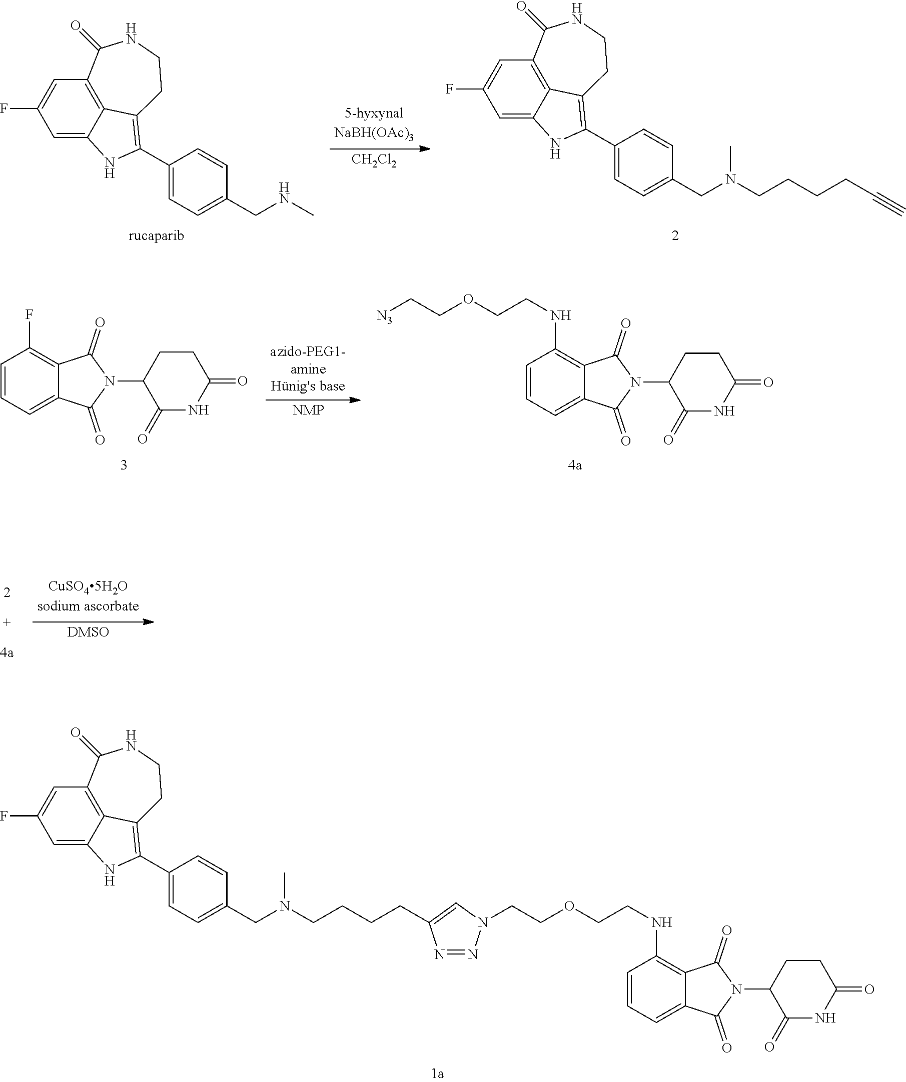

### Step 1. Synthesis of C6-Alkynylrucaparib (2)

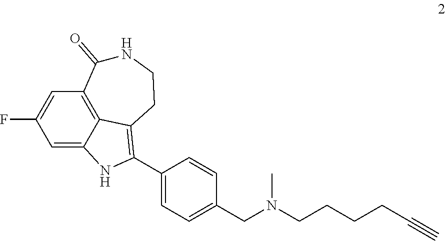 [SMILES: C#CCCCCN(C)Cc1ccc(-c2[nH]c3cc(F)cc4c3c2CCNC4=O)cc1]

To a solution of rucaparib (10.0 mg, 31.0 μmol, 1 equiv) and sodium triacetoxyborohydride (328 mg, 1.55 mmol, 50 equiv) in methylene chloride (3 mL) was added 5-hexynal (14.9 mg, 155 μmol, 5 equiv) under argon. After stirring at 23° C. for 1 h, the mixture was washed with saturated sodium bicarbonate solution followed by brine, dried with anhydrous sodium sulfate, concentrated, and purified by silica gel flash column chromatography to give 2 (11.4 mg, 74% yield) as a white solid. ¹H NMR (400 MHz, CDCl₃) δ 8.49 (br, 1H), 7.74 (dd, J=10.7, 2.1 Hz, 1H), 7.47 (d, J=8.2 Hz, 2H), 7.44 (d, J=8.2 Hz, 2H), 7.23 (dd, J=10.7, 2.1 Hz, 1H), 6.59 (t, J=5.8 Hz, 1H), 3.62-3.55 (m, 2H), 3.54 (s, 2H), 3.17-3.15 (m, 2H), 2.42 (t, J=7.0 Hz, 2H), 2.22 (s, 3H), 2.22-2.19 (m, 2H), 1.95 (t, J=2.5 Hz, 1H), 1.69-1.62 (m, 2H), 1.61-1.54 (m, 2H); ¹³C NMR (101 MHz, CDCl₃) δ 170.4, 159.6 (d, J=238.6 Hz), 139.6, 136.6 (d, J=11.9 Hz), 135.5 (d, J=3.5 Hz), 130.5, 129.7, 127.8, 125.2 (d, J=8.9 Hz), 123.9, 112.4, 111.9 (d, J=25.9 Hz), 101.4 (d, J=26.4 Hz), 84.6, 68.6, 62.1, 56.9, 43.2, 42.3, 29.0, 26.5, 26.4, 18.4; ¹⁹F NMR (376 MHz, CDCl₃) δ−119.9; MS (ESI)⁺ calculated for C₂₅H₂₇FN₃O (M+H)+404.2, found 404.1.

### Step 2. Synthesis of Azido-PEG1-Pomalidomides (4a)

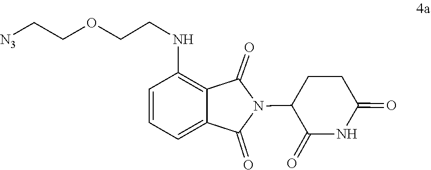 [SMILES: [N-]=[N+]=NCCOCCNc1cccc2c1C(=O)N(C1CCC(=O)NC1=O)C2=O]

To a solution of 2-(2,6-dioxo-piperidin-3-yl)-4-fluoroisoindoline-1,3-dione (3, 17.3 mg, 62.5 μmol, 1 equiv) and 2-(2-azidoethoxy)-ethan-1-amine (9 mg, 68.7 μmol, 1.1 equiv) in N-methyl-2-pyrrolidone (2 mL) was added Hünig's base (16.2 mg, 125 mol, 21.8 μL, 2 equiv). After stirring at 90° C. for 24 h, the mixture was concentrated and purified by silica gel flash column chromatography to give 4a as a yellow solid. (23.0 mg, 95% yield). ¹H NMR (400 MHz, CDCl₃) δ 8.13 (br, 1H), 7.50 (dd, J=8.5, 7.5 Hz, 1H), 7.11 (d, J=7.5 Hz, 1H), 6.94 (d, J=8.5 Hz, 1H), 6.49 (t, J=5.6 Hz, 1H), 4.94-4.90 (m, 1H), 3.72 (t, J=5.4 Hz, 2H), 3.68 (t, J=5.4 Hz, 2H), 3.50 (dd, J=5.5, 5.6 Hz, 2H), 3.41 (t, J=5.5 Hz, 2H), 2.98-2.68 (m, 3H), 2.26-2.05 (m, 1H); ¹³C NMR (101 MHz, CDCl₃) δ 171.0, 169.3, 168.3, 167.6, 146.8, 136.1, 132.5, 122.8, 116.8, 111.8, 70.1, 69.7, 50.7, 48.9, 42.4, 31.4, 22.8; MS (ESI)⁺ calculated for C₁₇H₁₉N₆O₅ (M+H)+387.1, found 387.1.

### Step 3. Synthesis of Rucaparib-Triazole-PEG1-Pomalidomide (1a)

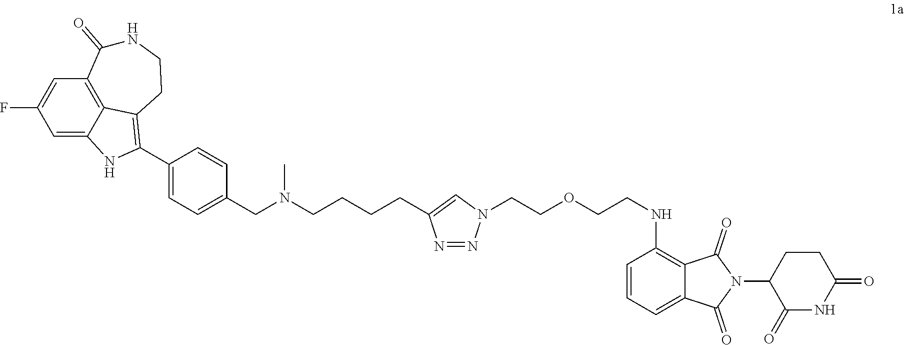 [SMILES: CN(CCCCc1cn(CCOCCNc2cccc3c2C(=O)N(C2CCC(=O)NC2=O)C3=O)nn1)Cc1ccc(-c2[nH]c3cc(F)cc4c3c2CCNC4=O)cc1]

To a solution of 2 (4.0 mg, 10 μmol, 1.0 equiv) and 4a (11 μmol, 1.1 equiv) in dimethyl sulfoxide (0.2 mL) was added copper(II) sulfate pentahydrate (5.0 mg, 20 μmmol, 2.0 equiv) and sodium ascorbate (8.0 mg, 40 μmol, 4.0 equiv). After stirring at 80° C. for 2 h, the mixture was concentrated and purified by preparative HPLC to give 5a as a yellow solid (4.6 mg, 59% yield). ¹H NMR (400 MHz, CD₃OD, selected signals) δ 7.72 (d, J=8.1 Hz, 2H), 7.68 (s, 1H), 7.61 (d, J=8.1 Hz, 2H), 7.55-7.48 (m, 2H), 7.33 (dd, J=8.9, 1.8 Hz, 1H), 7.05 (d, J=4.6 Hz, 1H), 7.03 (d, J=3.1 Hz, 1H), 5.07 (dd, J=12.4, 5.4 Hz, 1H), 4.55 (t, J=4.7 Hz, 2H), 3.87 (t, J=4.7 Hz, 2H), 3.66 (t, J=4.9 Hz, 2H), 3.59-3.51 (m, 2H), 3.46-3.43 (m, 2H), 3.35 (s, 2H); ¹⁹F NMR (376 MHz, CD₃OD) δ−119.5; MS (ESI)⁺ calculated for C₄₂H₄₅FN₉O₆ (M+H)⁺790.4, found 790.4.

### Rucaparib-Triazole-PEG2-Pomalidomide (1b)

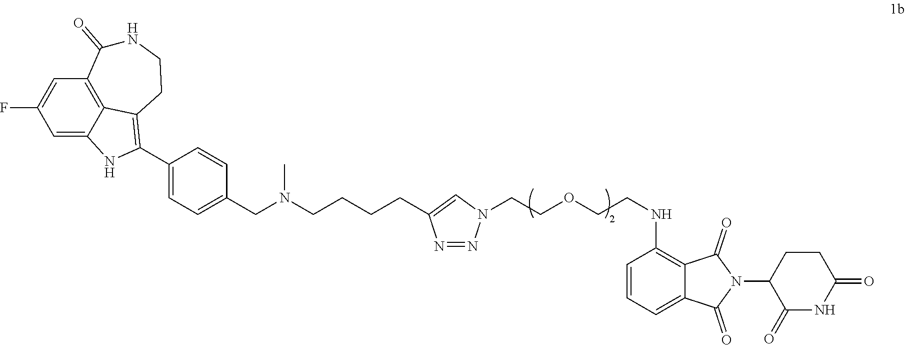

Prepared by essentially the same method as that for 1a. ¹H NMR (400 MHz, CD₃OD, selected signals) δ 7.77 (s, 1H), 7.72 (d, J=8.3 Hz, 2H), 7.61 (d, J=8.3 Hz, 2H), 7.55-7.50 (m, 2H), 7.32 (dd, J=8.9, 2.3 Hz, 1H), 7.04 (d, J=6.0 Hz, 1H), 7.02 (d, J=4.5 Hz, 1H), 5.03 (dd, J=12.6, 5.5 Hz, 1H), 4.52 (t, J=4.0 Hz, 2H), 4.47-4.41 (m, 1H), 4.31-4.25 (m, 1H), 3.88 (t, J=4.0 Hz, 2H), 3.66 (t, J=4.0 Hz, 2H), 3.63-3.61 (m, 4H), 3.57-3.51 (m, 2H), 3.43 (t, J=4.0 Hz, 2H); ¹⁹F NMR (376 MHz, CD₃OD) δ−119.5; MS (ESI)⁺ calculated for C₄₄H₄₉FN₉O₆ (M+H)⁺834.4, found 834.3.

### Rucaparib-Triazole-PEG3-Pomalidomide (1c)

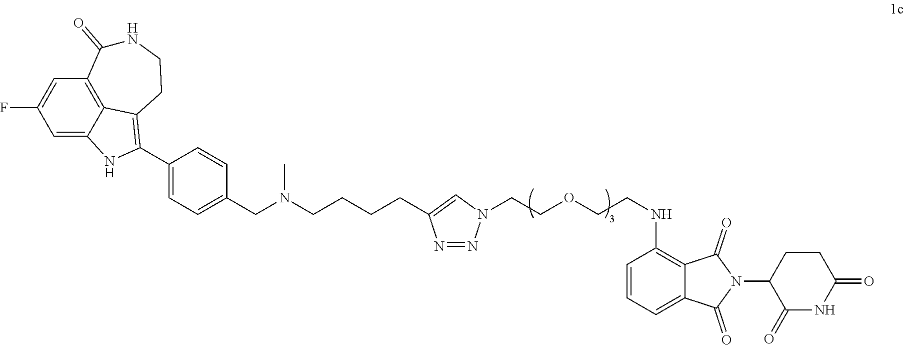

Prepared by essentially the same method as that for Ta. ¹H NMR (400 MHz, CDCl₃, selected signals) δ 11.9 (br, 1H), 9.53 (br, 1H), 8.85 (br, 1H), 8.19 (br, 1H), 7.71 (d, J=8.0 Hz, 1H), 7.64-7.61 (m, 1H), 7.56-7.43 (m, 4H), 7.33 (d, J=8.1 Hz, 1H), 7.04 (d, J=7.1 Hz, 1H), 6.86 (d, J=8.5 Hz, 1H), 4.90 (dd, J=11.4, 5.5 Hz, 1H), 4.50-4.46 (m, 2H), 4.22-4.13 (m, 2H), 3.85 (t, J=4.5 Hz, 2H), 3.69 (t, J=4.9 Hz, 2H); ¹⁹F NMR (376 MHz, CDCl₃) δ−119.7. MS (ESI)⁺ calculated for C₄₆H₅₃FN₉O₆ (M+H)⁺878.4, found 878.1.

### Rucaparib-Triazole-PEG4-Pomalidomide (1d)

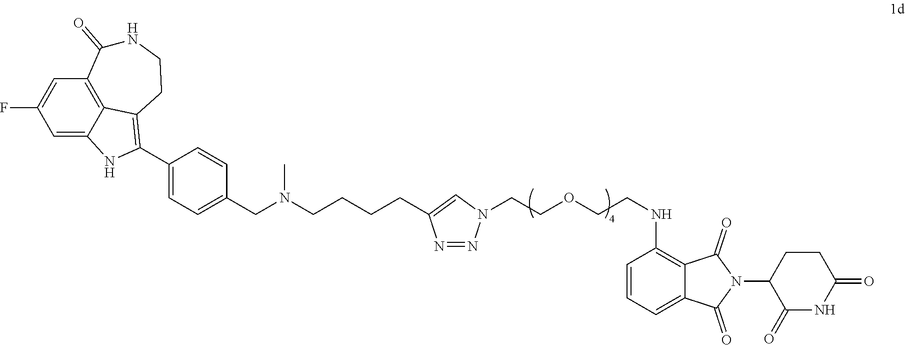

Prepared by essentially the same method as that for 1a. ¹H NMR (400 MHz, CD₃OD, selected signals) δ 7.78 (s, 1H), 7.70 (d, J=8.2 Hz, 2H), 7.59 (d, J=8.2 Hz, 2H), 7.55-7.45 (m, 2H), 7.30 (dd, J=8.9, 2.3 Hz, 1H), 7.00 (d, J=8.7 Hz, 1H), 6.98 (d, J=7.1 Hz, 1H), 5.01 (dd, J=12.5, 5.5 Hz, 1H), 4.48 (t, J=6.0 Hz, 2H), 3.81 (t, J=6.0 Hz, 2H), 3.65 (t, J=6.0 Hz, 2H); ¹⁹F NMR (376 MHz, CD₃OD) δ−121.6; MS (ESI)⁺ calculated for C₄₈H₅₇FN₉O₆ (M+H)⁺922.4, found 922.4.

### Rucaparib-Triazole-PEG5-Pomalidomide (1e)

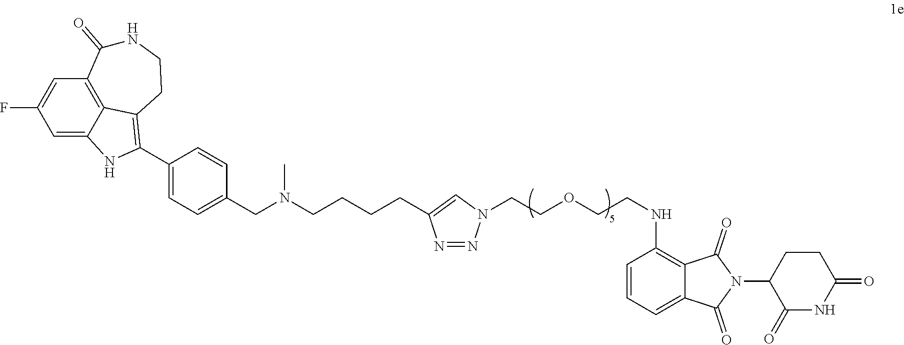

Prepared by essentially the same method as that for 1a. ¹⁹F NMR (376 MHz, CD₃OD) δ−121.5; MS (ESI)⁺ calculated for C₅₀H₆₁FN₉O₁₀ (M+H)⁺966.4, found 966.4.

### Rucaparib-Triazole-PEG3-VHL-Ligand (5a)

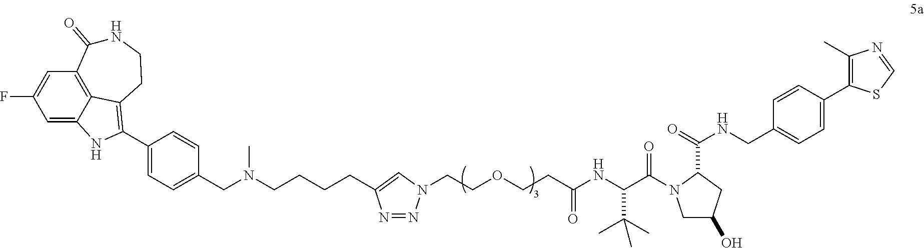

Prepared by essentially the same method as that for 1a except using the amide coupling product of (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide (J. Med. Chem., 2018, 61, 583-598) and azido-PEG3-acid instead of 4a. ¹H NMR (400 MHz, CD₃OD, selected signals) δ 8.89 (s, 1H), 7.80 (s, 1H), 7.75 (d, J=8.3 Hz, 2H), 7.64 (d, J=8.3 Hz, 2H), 7.54 (dd, J=8.9, 2.3 Hz, 1H), 7.45 (d, J=8.0 Hz, 2H), 7.39 (d, J=8.0 Hz, 2H), 7.33 (dd, J=8.9, 2.3 Hz, 1H); ¹⁹F NMR (376 MHz, CD₃OD) δ−121.7; MS (ESI)⁺ calculated for C₅₆H₇₂FN₁₀O₈S (M+H)⁺1063.5, found 1063.1.

### Rucaparib-Triazole-PEG4-VHL-Ligand (5b)

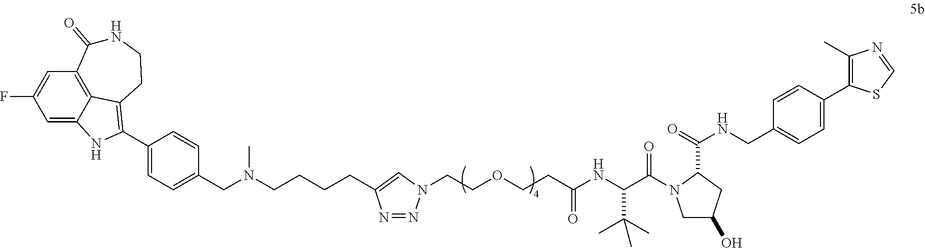

Prepared by essentially the same method as that for 5a. ¹H NMR (400 MHz, CD₃OD, selected signals) δ 8.89 (s, 1H), 7.80 (s, 1H), 7.75 (d, J=8.1 Hz, 2H), 7.64 (d, J=8.1 Hz, 2H), 7.54 (dd, J=10.8, 2.1 Hz, 1H), 7.45 (d, J=8.1 Hz, 2H), 7.39 (d, J=8.1 Hz, 2H), 7.33 (dd, J=8.9, 2.1 Hz, 1H); ¹⁹F NMR (376 MHz, CDCl₃) δ−116.7; MS (ESI)⁺ calculated for C₅₈H₇₆FN₁₀O₉S (M+H)⁺1107.6, found 1108.1.

### Rucaparib-C5 Amide-PEG3-Pomalidomide (6a)

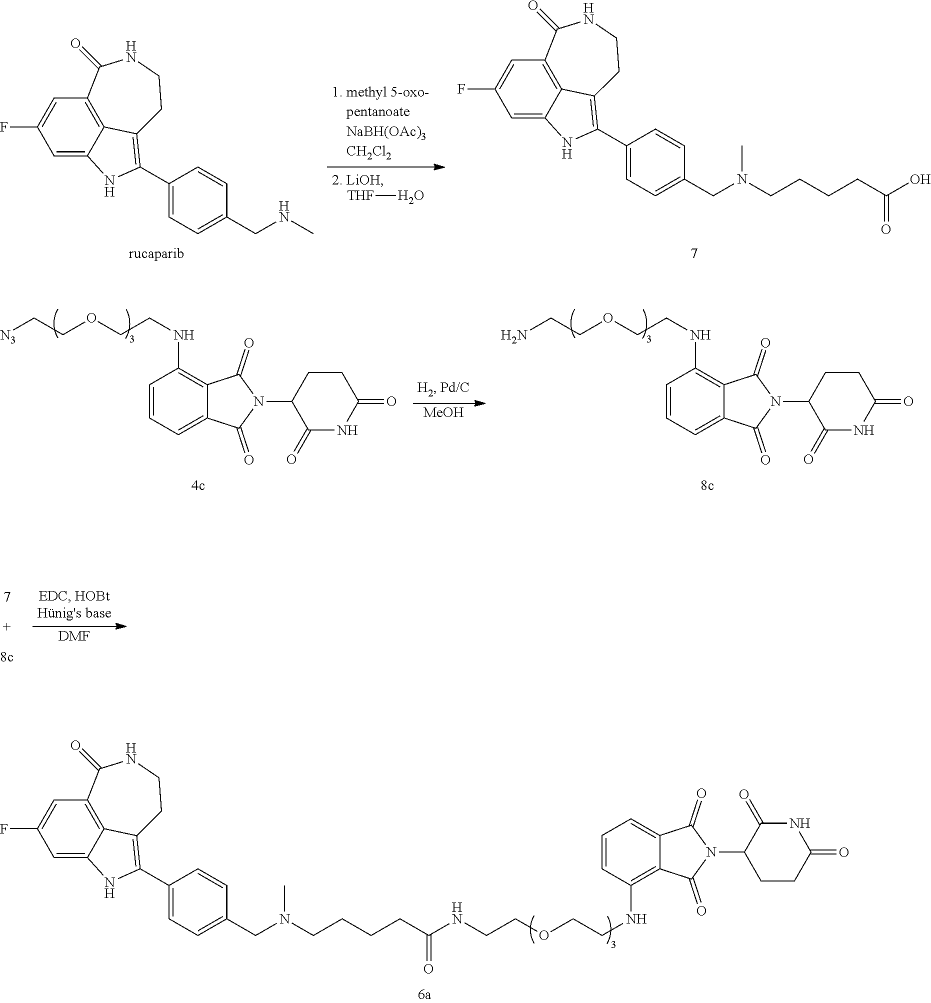

### Step 1. Synthesis of C5-Carboxylylrucaparib (7)

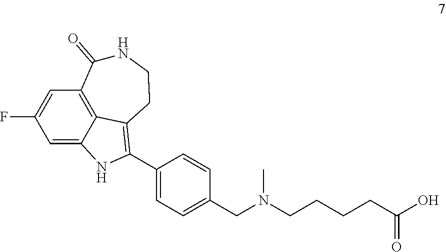 [SMILES: CN(CCCCC(=O)O)Cc1ccc(-c2[nH]c3cc(F)cc4c3c2CCNC4=O)cc1]

To a solution of rucaparib (10.0 mg, 31.0 μmol, 1 equiv) and sodium triacetoxyborohydride (328 mg, 1.55 mmol, 50 equiv) in methylene chloride (3 mL) was added methyl 5-oxo-pentanoate (20.2 mg, 155 μmol, 5 equiv) under argon. After stirring at 23° C. for 1 h, the mixture was washed with saturated sodium bicarbonate solution followed by brine, dried with anhydrous sodium sulfate, concentrated, and re-dissolved in tetrahydrofuran (0.5 mL). Aqueous lithium hydroxide solution (2M) was then added the reaction was stirred at 23° C. for 2 h before acidified, concentrated, and purified by reversal phase HPLC to give 7 as a white solid (7.0 mg, 54% yield). ¹H NMR (400 MHz, CD₃OD) δ 7.70 (d, J=8.2 Hz, 2H), 7.63 (d, J=8.2 Hz, 2H), 7.52 (dd, J=10.8, 2.2 Hz, 1H), 7.31 (dd, J=9.0, 2.2 Hz, 1H), 4.49 (d, J=11.6 Hz, 1H), 4.29 (d, J=11.6 Hz, 1H), 3.52 (d, J=4.2 Hz, 2H), 3.28-3.10 (m, 4H), 2.40 (t, J=7.1 Hz, 2H), 1.94-1.76 (m, 2H), 1.74-1.58 (m, 2H); ¹³C NMR (101 MHz, CD₃OD) δ 176.7, 172.4, 160.8 (d, J=236.7 Hz), 138.7 (d, J=12.2 Hz), 136.1 (d, J=3.4 Hz), 135.1, 132.7, 130.0, 129.7, 126.2 (d, J=8.9 Hz), 124.8, 114.0, 111.6 (d, J=26.3 Hz), 102.4 (d, J=26.3 Hz), 60.5, 56.8, 43.7, 40.0, 33.9, 30.0, 24.7, 22.8; ¹⁹F NMR (376 MHz, CD₃OD) δ−121.42; MS (ESI)⁺ calculated for C₂₄H₂₇FN₃O₃ (M+H)⁺424.2, found 424.2.

### Step 2. Synthesis of Amino-PEG3-Pomalidomides (8c)

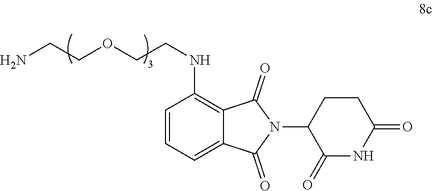

A solution of 4c (20.0 mg) and Pd/C (10% w/w, 2.0 mg) in methanol (0.2 mL) was stirred under hydrogen (1 atm) and stirred at 23° C. for 30 min and then filtrated and concentrated to give crude 8c that was used directly in the next step.

### Step 3. Synthesis of Rucaparib-C5 Amide-PEG3-Pomalidomide (6a)

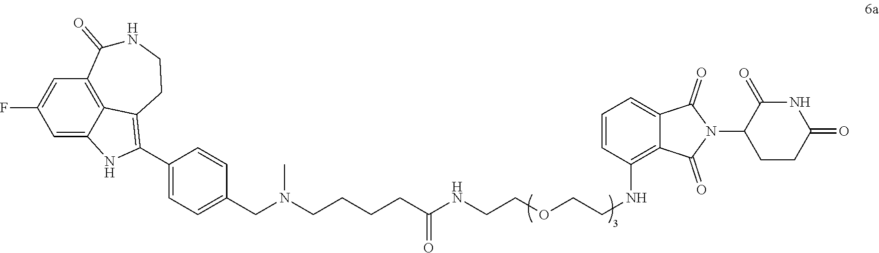

To a solution of 7 (7.0 mg, 16.0 μmol, 1.0 equiv) and 8c (8.6 mg, 19.2 μmol, 1.2 equiv) in anhydrous N,N-dimethylformamide (0.4 mL) was added N-(3-dimethylaminopropyl)-N′-ethylcarbodiimide hydrochloride (EDCI, 4.6 mg, 24.0 μmol, 1.5 equiv), 1-hydroxybenzotriazole hydrate (HOBt, 3.2 mg, 24.0 μmol, 1.5 equiv) and Hünig's base (10.3 mg, 80.0 μmol, 5.0 equiv). After stirring at 23° C. overnight, the reaction mixture was extracted with ethyl acetate, washed with saturated sodium bicarbonate solution followed by brine, dried with anhydrous sodium sulfate, concentrated, and purified by HPLC to give 6a as a yellow solid (5.1 mg, 37% yield). ¹H NMR (400 MHz, CD₃OD, selected signals) δ 7.73 (d, J=8.3 Hz, 2H), 7.64 (d, J=8.3 Hz, 2H), 7.56-7.48 (m, 2H), 7.33 (dd, J=9.0, 2.3 Hz, 1H), 7.04 (d, J=8.7 Hz, 1H), 7.01 (d, J=7.2 Hz, 1H), 5.04 (dd, J=12.5, 5.5 Hz, 1H), 4.47 (d, J=13.0 Hz, 1H), 4.28 (d, J=13.0 Hz, 1H), 3.68 (t, J=5.2 Hz, 2H); ¹⁹F NMR (376 MHz, CD₃OD) δ−121.5; MS (ESI)⁺ calculated for C₄₅H₅₃FN₇O₉ (M+H)⁺854.4, found 854.4.

### Rucaparib-C6 Amide-PEG3-Pomalidomide (6b)

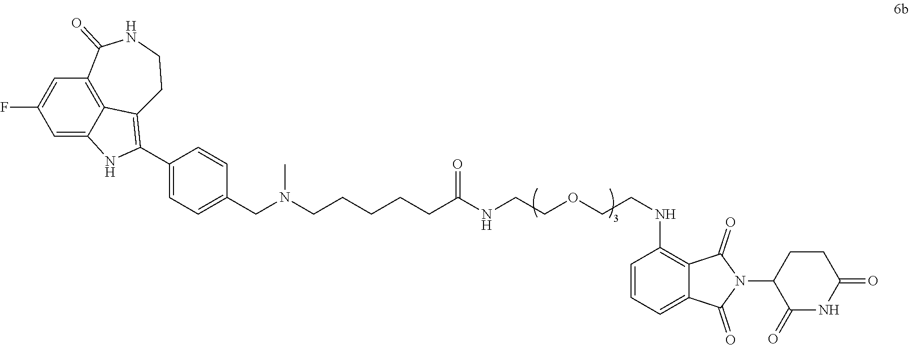

Prepared by essentially the same method as that for 6a. ¹⁹F NMR (376 MHz, CD₃OD) δ−121.6; MS (ESI)⁺ calculated for C₄₆H₅₅FN₇O₉ (M+H)⁺868.4, found 868.4.

### Rucaparib-PEG5-Pomalidomide (9a)

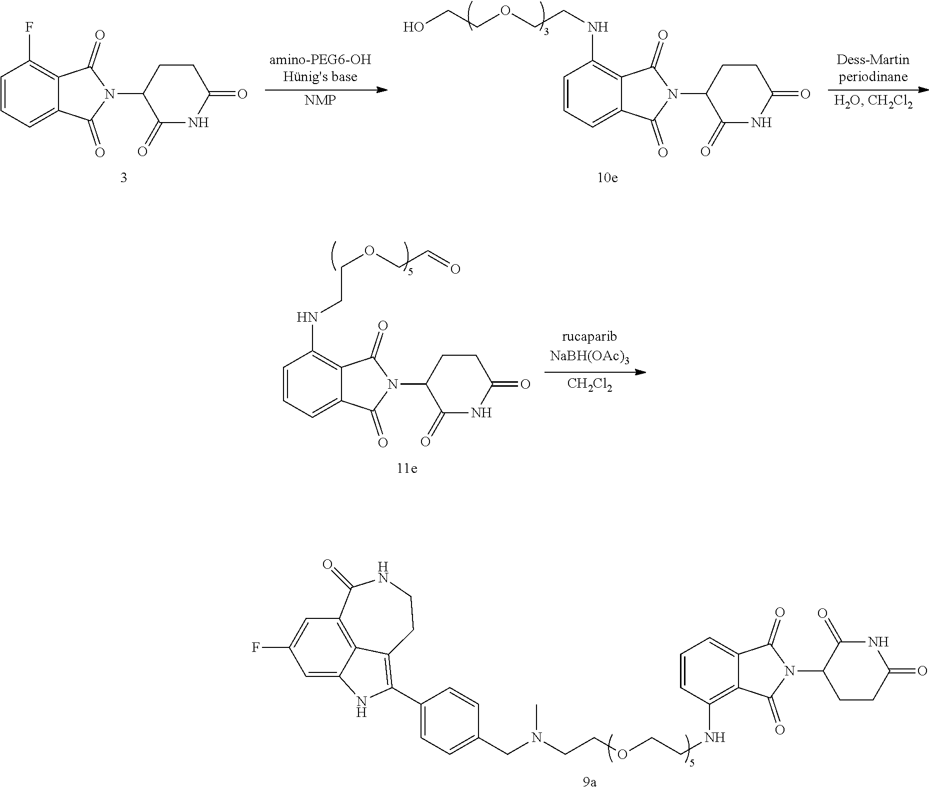

### Step 1. Synthesis of Hydroxy-PEG5-Pomalidomides (10e)

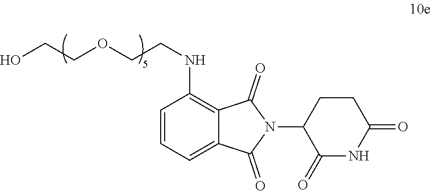

To a solution of 3 (30.0 mg, 108.6 μmol, 1.0 equiv) and amino-PEG6-alcohol (60.1 mg, 217.2 μmol, 2.0 equiv) in N-methyl-2-pyrrolidone (2 mL) was added triethylamine (33.2 mg, 325.8 μmol, 3.0 equiv). After stirring at 90° C. for 24 h, the mixture was concentrated and purified by silica gel flash column chromatography to give 10e as a yellow solid. ¹H NMR (400 MHz, CDCl₃) δ 7.47 (dd, J=8.5, 7.1 Hz, 1H), 7.08 (d, J=7.1 Hz, 1H), 6.90 (d, J=8.5 Hz, 1H), 6.48 (t, J=5.4 Hz, 1H), 4.90 (dd, J=11.7, 5.3 Hz, 1H), 3.78-3.53 (m, 22H), 3.45 (dd, J=10.8, 5.4 Hz, 2H), 2.88-2.68 (m, 3H), 2.14-2.05 (m, 1H); ¹³C NMR (101 MHz, CDCl₃) δ 171.6, 169.3, 168.7, 167.7, 146.9, 136.1, 132.6, 116.9, 111.7, 110.4, 72.6, 70.9, 70.7, 70.6, 70.6, 70.5, 70.3, 69.5, 61.7, 49.0, 42.5, 31.5, 22.9; MS (ESI)⁺ calculated for C₂₅H₃₆N₃O₁₀ (M+H)⁺538.2, found 538.3.

### Step 2. Synthesis of Carbonyl-PEG5-Pomalidomides (I1e)

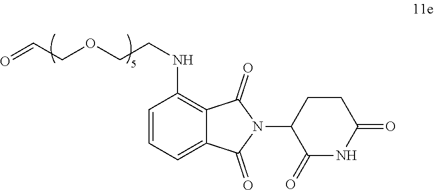

To a solution of 10e (50.0 mg, 93.0 μmol, 1.0 equiv) in methylene chloride (2 mL) was added Dess-Martin periodinane (59.2 mg, 139.5 μmol, 1.5 equiv) and water (2.0 mg, 111.6 μmol, 1.2 equiv) at 0° C. After stirring at 23° C. for 16 h, the mixture was washed with saturated sodium bicarbonate solution followed by brine, dried with anhydrous sodium sulfate, concentrated, and purified by silica gel flash chromatography to give 11e as a yellow solid (40.5 mg, 81% yield). ¹H NMR (400 MHz, CDCl₃) δ 9.70 (s, 1H), 8.51 (s, 1H), 7.47 (t, J=7.0 Hz, 1H), 7.09 (d, J=6.9 Hz, 1H), 6.91 (d, J=8.4 Hz, 1H), 6.48 (br, 1H), 4.90 (dd, J=11.6, 5.1 Hz, 1H), 3.78-3.51 (m, 20H), 3.45 (d, J=4.9 Hz, 2H), 2.89-2.67 (m, 3H), 2.14-2.05 (m, 1H); ¹³C NMR (101 MHz, CDCl₃) δ 201.1, 171.4, 169.4, 168.6, 167.7, 146.9, 136.2, 132.6, 116.9, 111.8, 110.4, 76.9, 71.3, 70.9, 70.8, 70.7, 70.6, 69.6, 49.0, 42.5, 31.5, 22.9; MS (ESI)⁺ calculated for C₂₇H₃₈N₃O₁₁ (M+H)⁺536.2, found 536.2.

### Step 3. Synthesis of Rucaparib-PEG5-Pomalidomide (9a)

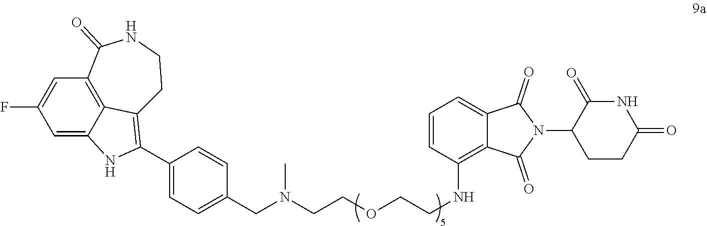

To a solution of rucaparib (5.0 mg, 15.4 μmol, 1.0 equiv) and 11e (10.0 mg, 18.6 μmol, 1.2 equiv) in methylene chloride (1 mL) was added sodium triacetoxyborohydride (163.9 mg, 773.1 μmol, 50.0 equiv) at 0° C. After stirring at 23° C. for 2 h, the mixture was washed with saturated sodium bicarbonate solution followed by brine, dried with anhydrous sodium sulfate, concentrated, and purified by HPLC to 9a as a yellow solid (11.7 mg, 90% yield). ¹H NMR (400 MHz, CD₃OD, selected signals) δ 7.72 (d, J=8.2 Hz, 2H), 7.65 (d, J=8.2 Hz, 2H), 7.52 (dd, J=10.8, 2.2 Hz, 1H), 7.49-7.43 (m, 1H), 7.31 (dd, J=8.9, 2.2 Hz, 1H), 6.99 (d, J=3.6 Hz, 1H), 6.97 (d, J=5.2 Hz, 1H), 5.02 (dd, J=12.7, 5.5 Hz, 1H), 4.54 (d, J=13.0 Hz, 1H), 4.35 (d, J=12.9 Hz, 1H), 3.86 (t, J=4.9 Hz, 2H); ¹⁹F NMR (376 MHz, CD₃OD) δ−121.5; MS (ESI)⁺ calculated for C₄₄H₅₂FN₆O₁₀ (M+H)⁺843.4, found 843.4.

### Rucaparib-PEG6-Pomalidomide (9b)

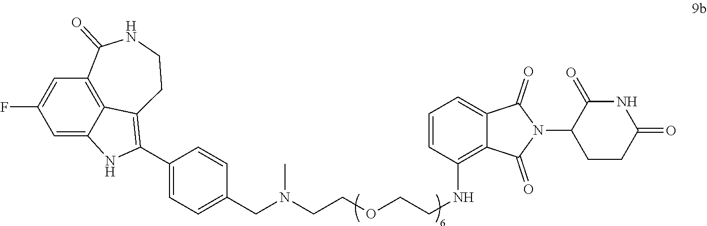

Prepared by essentially the same method as that for 9a. ¹⁹F NMR (376 MHz, CD₃OD) δ−121.5; MS (ESI)⁺ calculated for C₄₆H₅₆FN₆O₁₁ (M+H)⁺887.4, found 887.4.

### Rucaparib-PEG7-Pomalidomide (9c)

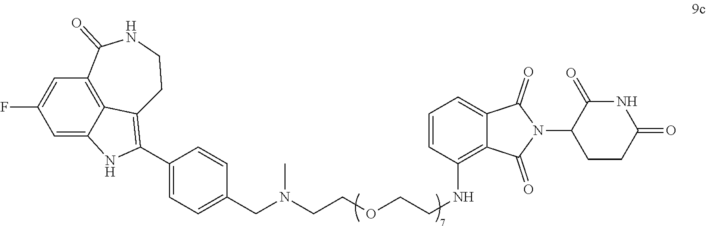

Prepared by essentially the same method as that for 9a. ¹⁹F NMR (376 MHz, CD₃OD) δ−121.6; MS (ESI)⁺ calculated for C₄₈H₆₀FN₆O₁₂ (M+H)⁺931.4, found 931.4.

### Rucaparib-(Indole)Triazole-PEG3-Pomalidomide (12a)

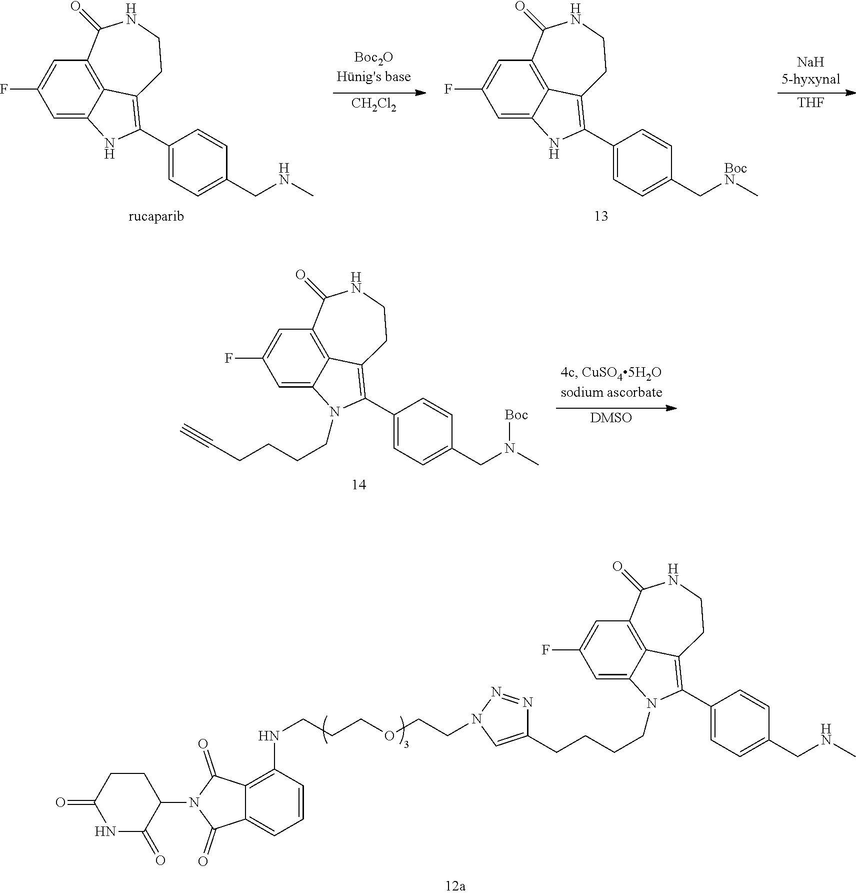

### Step 1. Synthesis of Boc-Rucaparib (13)

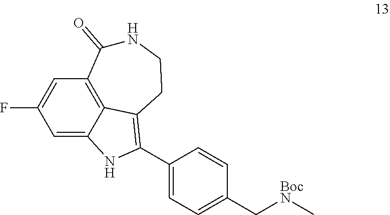 [SMILES: CN(Cc1ccc(-c2[nH]c3cc(F)cc4c3c2CCNC4=O)cc1)C(=O)OC(C)(C)C]

To a solution of rucaparib (20.0 mg, 62.0 μmol, 1 equiv) in methylene chloride (0.6 mL) was added di-tert-butyl dicarbonate (16.2 mg, 74.2 μmol, 1.2 equiv) and Hünig's base (12.0 mg 92.8 μmol, 1.5 equiv). After stirring at 23° C. for 30 min, the mixture was washed with saturated sodium bicarbonate solution followed by brine, dried with anhydrous sodium sulfate, concentrated, and purified by silica gel flash column chromatography to give 13 (23.3 mg, 89% yield). ¹H NMR (400 MHz, CD₃OD) δ 7.57 (d, J=8.0 Hz, 2H), 7.50 (dd, J=10.8, 2.3 Hz, 1H), 7.36 (d, J=8.2 Hz, 2H), 7.28 (dd, J=9.0, 2.3 Hz, 1H), 4.48 (s, 2H), 3.51 (d, J=4.6 Hz, 2H), 3.17-3.05 (m, 2H), 2.85 (s, 3H), 1.48 (d, J=10.4 Hz, 9H); ¹³C NMR (101 MHz, CD₃OD) δ 171.1, 171.1, 159.1 (d, J=235.9 Hz), 137.1 (d, J=12.0 Hz), 135.8 (d, J=3.4 Hz), 131.0, 127.9, 127.4 (d, J=18.0 Hz), 124.3 (d, J=8.9 Hz), 123.6, 111.5, 109.8 (d, J=26.2 Hz), 100.9 (d, J=26.3 Hz), 79.9, 51.9, 42.4, 33.2, 28.5, 27.3; MS (ESI)⁺ calculated for C₂₄H₂₇FN₃O₃ (M+H)⁺424.2, found 424.2.

### Step 2. Synthesis of Boc-(Indole)C6-Alkynylrucaparib (14)

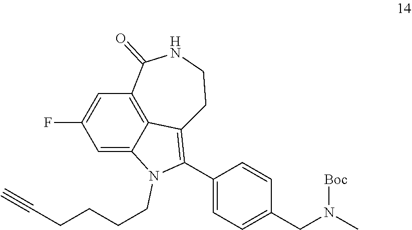 [SMILES: C#CCCCCn1c(-c2ccc(CN(C)C(=O)OC(C)(C)C)cc2)c2c3c(cc(F)cc31)C(=O)NCC2]

To a solution of 13 (23.3 mg, 55 μmol, 1.0 equiv) in anhydrous tetrahydrofuran (0.5 mL) under argon was added sodium hydride (60% w/w, 5.3 mg, 132.1 μmol, 2.4 equiv) at 0° C. After stirring for 30 min, 6-iodohex-1-yne (13.7 mg, 66.0 μmol, 1.2 equiv) was added dropwise and the reaction was warm to 40° C. slowly. After stirring for 48 h, the reaction was quenched with saturated sodium bicarbonate solution followed by brine, dried with anhydrous sodium sulfate, concentrated, and purified by preparative HPLC to give 14 as a yellow solid (18.8 mg, 68% yield). ¹H NMR (400 MHz, CD₃OD) δ 7.56 (dd, J=10.7, 2.1 Hz, 1H), 7.47 (dd, J=9.6, 2.2 Hz, 1H), 7.45-7.37 (m, 4H), 4.54 (s, 2H), 4.17 (t, J=7.3 Hz, 2H), 3.54-3.43 (m, 2H), 2.89 (dd, J=11.0, 7.5 Hz, 5H), 2.15 (t, J=2.5 Hz, 1H), 1.97 (td, J=6.9, 2.6 Hz, 2H), 1.68 (dt, J=14.9, 7.4 Hz, 2H), 1.49 (d, J=18.3 Hz, 9H), 1.33-1.19 (m, 2H); ¹³C NMR (101 MHz, CD₃OD) δ 172.3, 172.3, 160.5 (d, J=236.2 Hz), 139.9, 139.8, 139.0 (d, J=11.5 Hz), 131.9, 131.3, 128.7 (d, J=17.2 Hz), 126.0 (d, J=8.9 Hz), 123.9, 114.5, 111.5 (d, J=26.2 Hz), 101.8 (d, J=26.9 Hz), 84.3, 81.4, 70.0, 53.4, 52.6, 49.0, 44.5, 43.8, 34.8, 29.6, 29.3, 28.7, 26.5, 18.4; ¹⁹F NMR (376 MHz, CD₃OD) δ−122.4; MS (ESI)⁺ calculated for C₃₀H₃₅FN₃O₃ (M+H)⁺504.3, found 504.3.

### Step 3. Rucaparib-(Indole)Triazole-PEG3-Pomalidomide (12a)

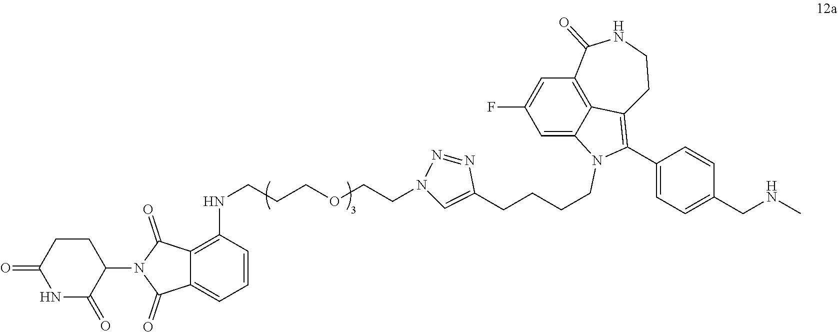

Prepared by essentially the same method as that for 1a except using 14 and 4c instead of 2 and 4a. ¹H NMR (400 MHz, CD₃OD, selected signals) δ 7.63 (s, 1H), 7.60 (d, J=8.2 Hz, 2H), 7.57 (dd, J=10.7, 2.3 Hz, 1H), 7.52-7.43 (m, 4H), 7.02 (d, J=4.4 Hz, 1H), 7.00 (d, J=2.9 Hz, 1H), 5.01 (dd, J=12.7, 5.5 Hz, 1H), 4.47 (d, J=6.0 Hz, 2H), 4.28 (s, 2H), 4.15 (t, J 10=7.1 Hz, 2H), 3.81 (d, J=6.0 Hz, 2H), 3.63 (t, J=5.2 Hz, 2H); ¹⁹F NMR (376 MHz, CD₃OD) δ−120.9; MS (ESI)⁺ calculated for C₄₆H₅₃FN₉O₈ (M+H)⁺878.4, found 878.3.

### Rucaparib-(Indole)Triazole-PEG4-Pomalidomide (12b)

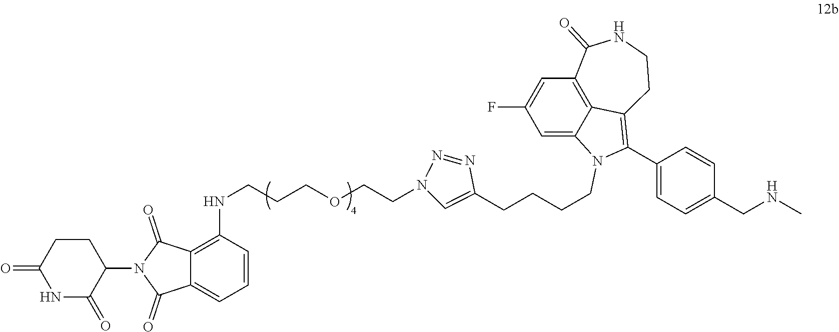

Prepared by essentially the same method as that for 12a. ¹⁹F NMR (376 MHz, CD₃OD) δ−121.4; MS (ESI)⁺ calculated for C₄₈H₅₇FN₉O₉ (M+H)⁺922.4, found 922.4.

### Veliparib-PEG5-Pomalidomide (15a)

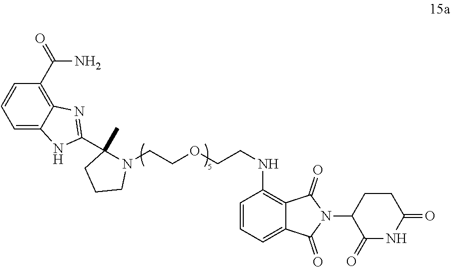

Prepared by essentially the same method as that for 9a except using veliparib instead of rucaparib. MS (ESI)⁺ calculated for C₃₈H₅₀N₇O₁₀ (M+H)⁺764.4, found 764.4.

### Veliparib-PEG6-Pomalidomide (15b)

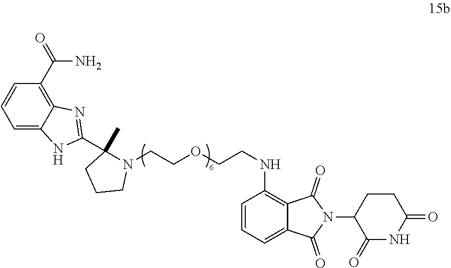

Prepared by essentially the same method as that for 15a. MS (ESI)⁺ calculated for C₄₀H₅₄N₇O₁₁ (M+H)⁺808.4, found 808.4.

### Veliparib-PEG7-Pomalidomide (15c)

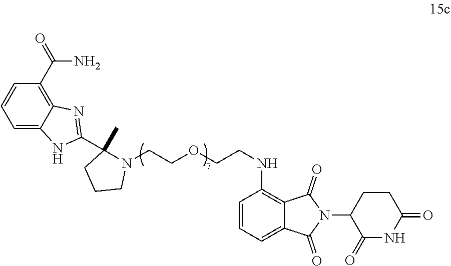

Prepared by essentially the same method as that for 15a. MS (ESI)⁺ calculated for C₄₂H₅₈N₇O₁₂ (M+H)⁺852.4, found 808.4.

### Niraparib-PEG5-Pomalidomide (16a)

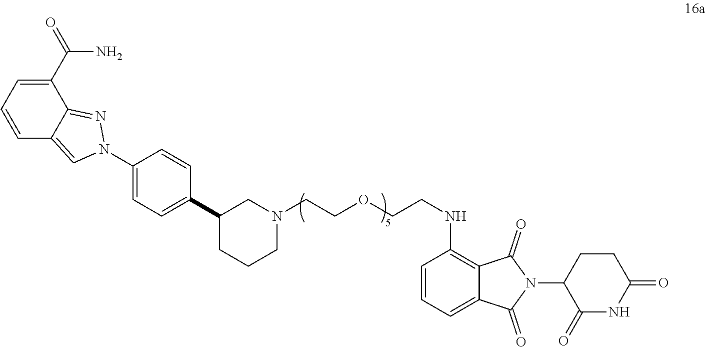

Prepared by essentially the same method as that for 9a except using niraparib instead of rucaparib. MS (ESI)⁺ calculated for C₄₄H₅₄N₇O₁₀ (M+H)⁺840.4, found 840.4.

### Niraparib-PEG6-Pomalidomide (16b)

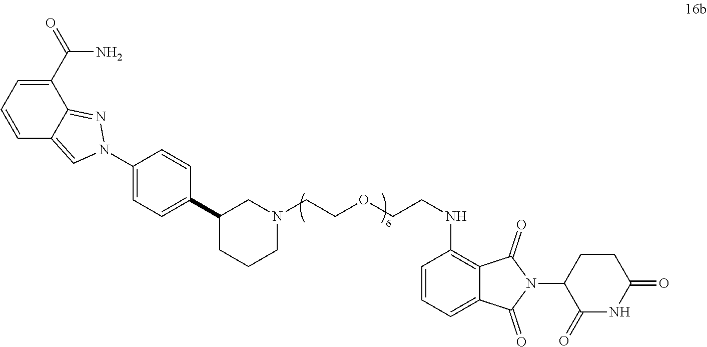

Prepared by essentially the same method as that for 16a. MS (ESI)⁺ calculated for C₄₆H₅₈N₇O₁₁ (M+H)⁺884.4, found 884.4.

### Niraparib-PEG7-Pomalidomide (16c)

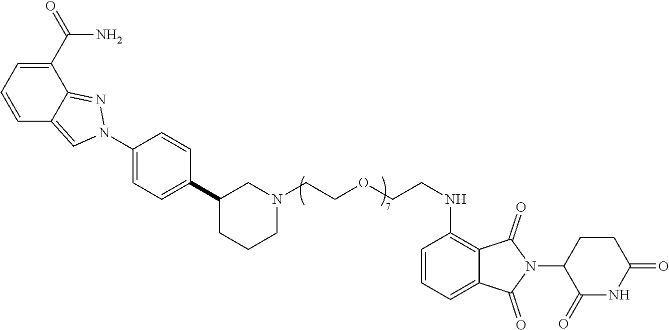

Prepared by essentially the same method as that for 16a. MS (ESI)⁺ calculated for C₄₈H₆₂N₇O₁₂ (M+H)⁺928.5, found 928.4.

### IWR-Triazole-PEG2-Pomalidomide (17a)

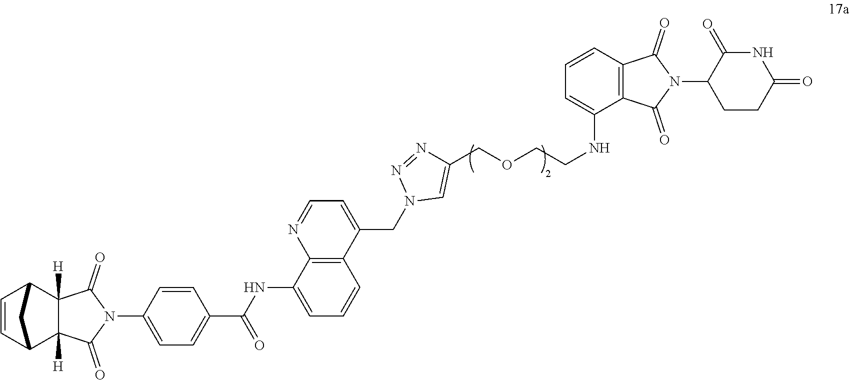

Prepared by essentially the same method as that for 1a except using IWR-2-azide derived from IWR-2-OH (Nat. Chem. Biol. 2009, 5, 100-107) and alkyne-PEG2-amine instead of 2 and 3. MS (ESI)⁺ calculated for C₄₆H₄₂N₉O₉ (M+H)⁺, 864.3, found 864.3.

### IWR-Triazole-PEG3-Pomalidomide (17b)

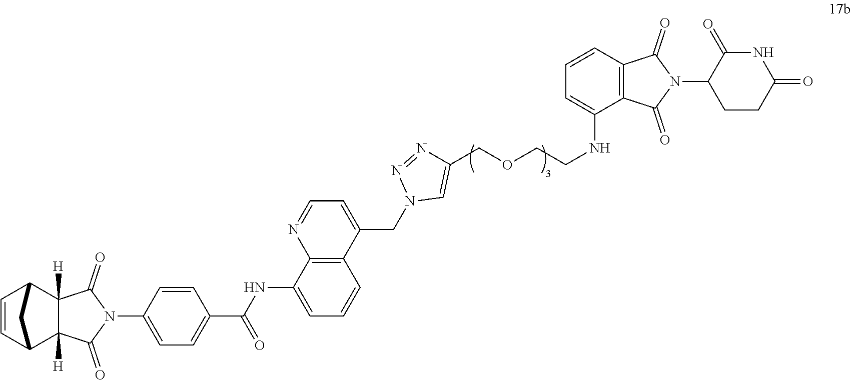

Prepared by essentially the same method as that for 17a. MS (ESI)⁺ calculated for C₄₈H₄₆N₉O₁₀ (M+H)⁺, 908.3, found 908.3

### IWR-Triazole-PEG4-Pomalidomide (17c)

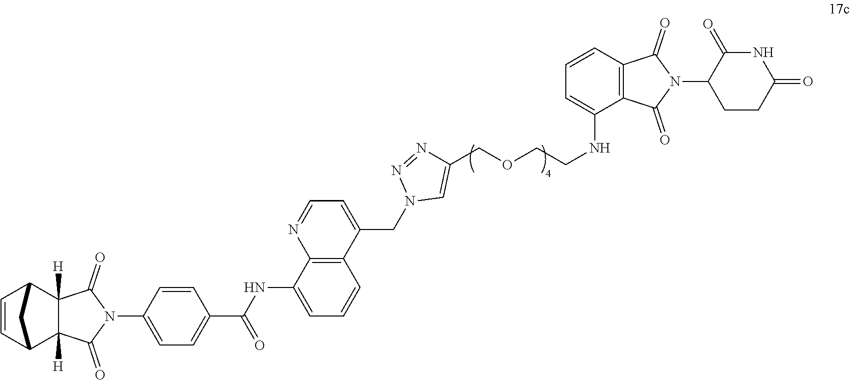

Prepared by essentially the same method as that for 17a. MS (ESI)⁺ calculated for C₅₀H₅₀N₉O₁₁ (M+H)⁺, 952.4, found 952.3.

### IWR-Triazole-PEG1-Pomalidomide (17d)

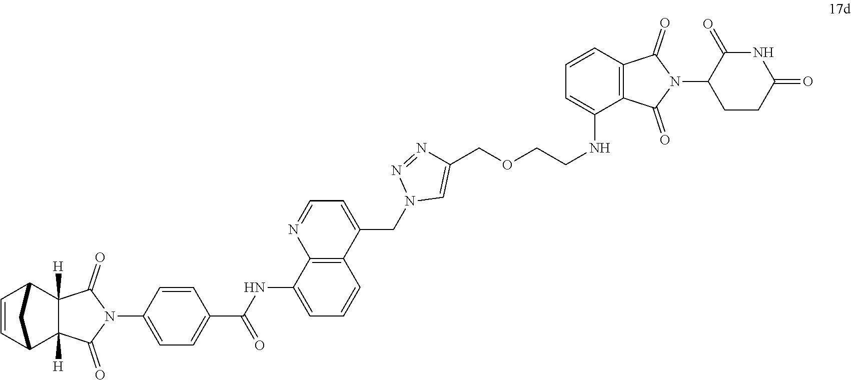

Prepared by essentially the same method as that for 17a. MS (ESI)⁺ calculated for C₄₄H₃₈N₉O₈ (M+H)⁺, 820.3, found 820.3.

### IWR-Triazole-PEG5-Pomalidomide (17e)

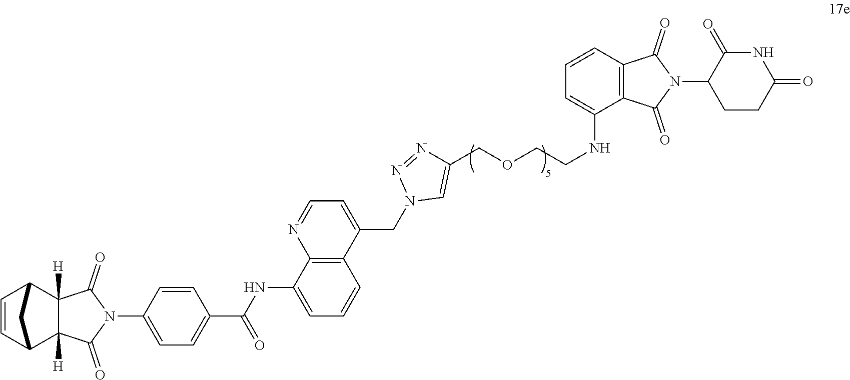

Prepared by essentially the same method as that for 17a. MS (ESI)⁺ calculated for C₅₂H₅₄N₉O₁₂ (M+H)⁺, 996.4, found 996.4.

### IWR-Triazole-PEG6-Pomalidomide (17f)

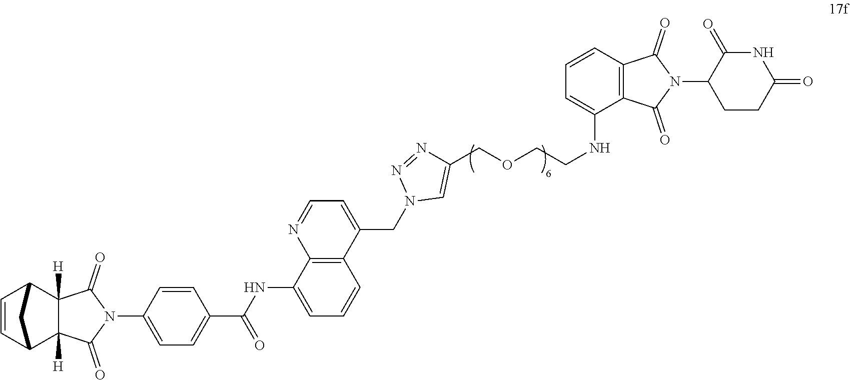

Prepared by essentially the same method as that for 17a. MS (ESI)⁺ calculated for C₅₄H₅₈N₉O₁₃ (M+H)⁺, 1040.4, found 1040.4.

### IWR-PEG2-VHL-Ligand (18a)

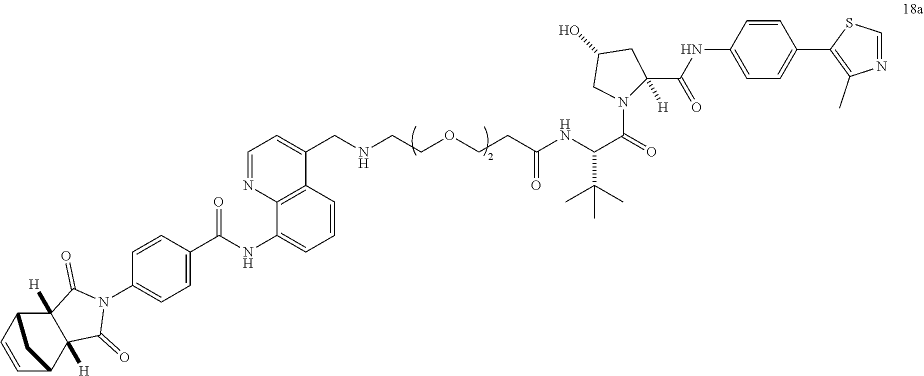

Prepared by essentially the same method as that for 6a. MS (ESI)⁺ calculated for C₅₅H₆₃N₈O₉S (M+H)⁺, 1011.4, found 1011.4.

### IWR-PEG3-VHL-Ligand (18b)

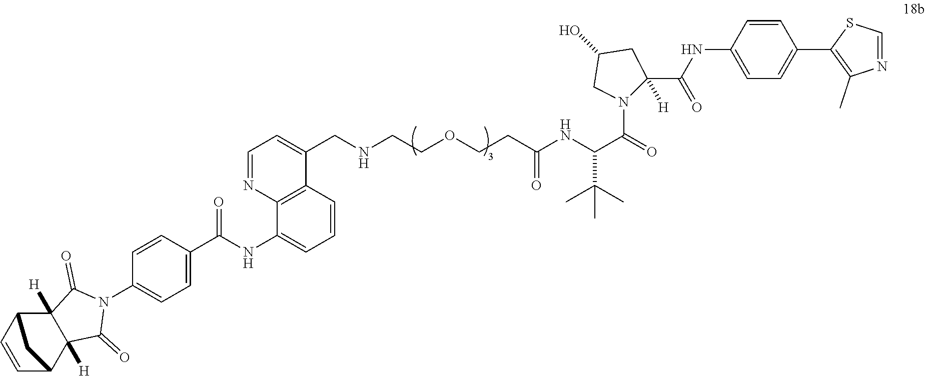

Prepared by essentially the same method as that for 18a. MS (ESI)⁺ calculated for C₅₇H₆₇N₈O₁₀S (M+H)⁺, 1055.5, found 1055.4.

### IWR-PEG4-VHL-Ligand (18c)

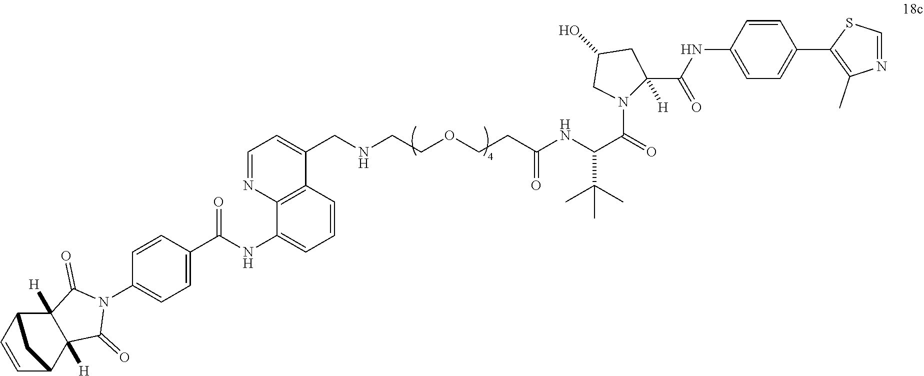

Prepared by essentially the same method as that for 18a. MS (ESI)⁺ calculated for C₅₉H₇₁N₈O₁₁S (M+H)⁺, 1099.5, found 1099.4.

### IWR-PEG1-VHL-Ligand (18d)

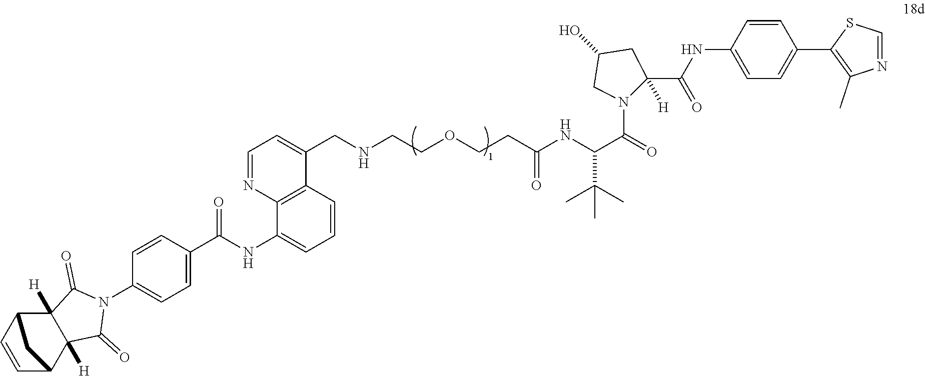

Prepared by essentially the same method as that for 18a. MS (ESI)⁺ calculated for C₅₃H₅₉N₈O₈S (M+H)⁺, 967.4, found 967.4.

### IWR-PEG5-VHL-Ligand (18e)

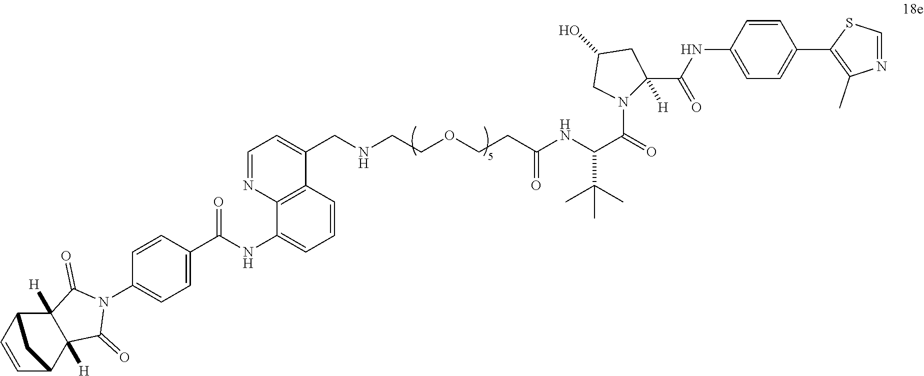

Prepared by essentially the same method as that for 18a. MS (ESI)⁺ calculated for C₆₁H₇₅N₈O₁₂S (M+H)⁺, 1143.5, found 1143.5.

### IWR-PEG6-VHL-ligand (18f)

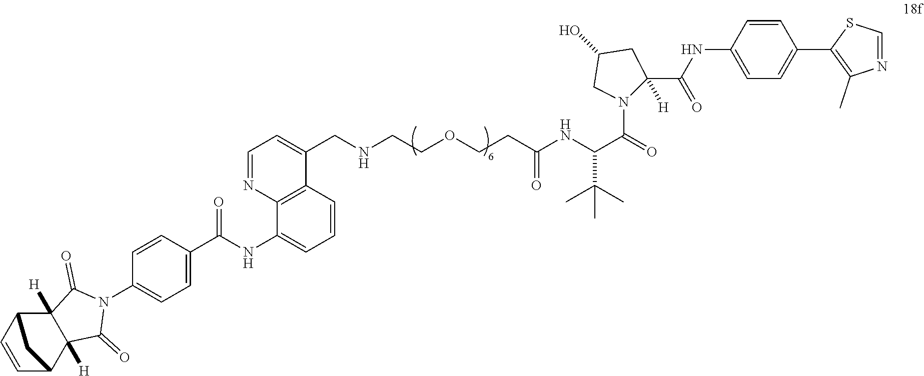

Prepared by essentially the same method as that for 18a. MS (ESI)⁺ calculated for C₆₃H₇₉N₈O₁₃S (M+H)⁺, 1187.5, found 1187.5.

### IWR-PEG3-Pomalidomide (19a)

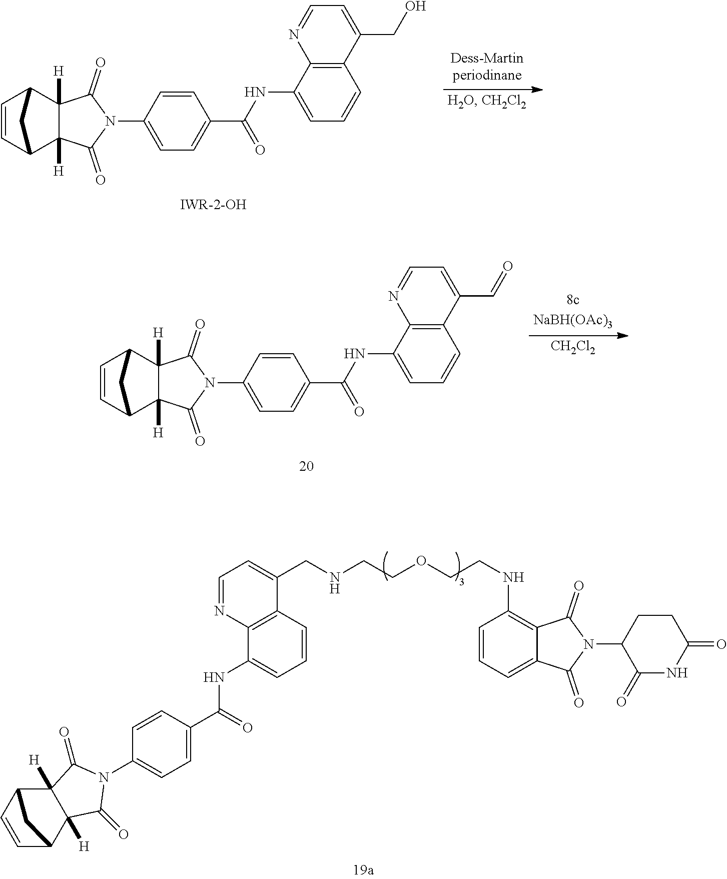

### Step 1. Synthesis of IWR-2-Aldehyde (20)

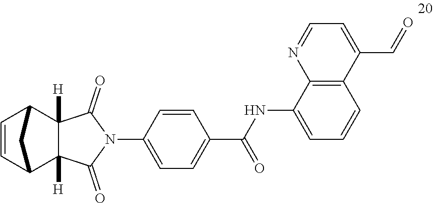

Prepared from IWR-2-OH according to a reported method (EBioMedicine 2019, 39, 145-158). MS (ESI)⁺ calculated for C₂₆H₂₀N₃O₄ (M+H)⁺438.1, found 438.1.

### Step 2. Synthesis of IWR-PEG3-Pomalidomide (19a)

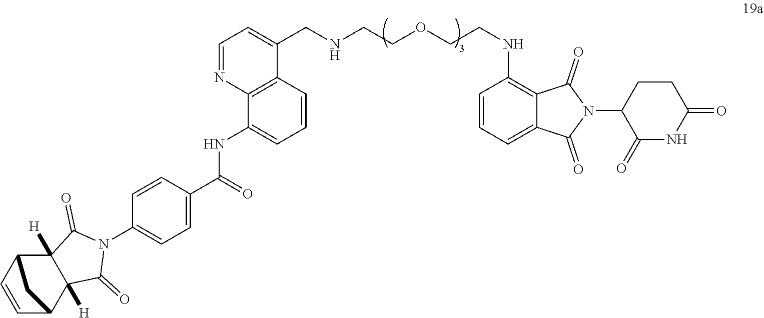

Prepared by essentially the same method as that for 9a except using 20 and 8c instead of I1e and rucaparib. MS (ESI)⁺ calculated for C₄₇H₄₈N₇O₁₀ (M+H)⁺870.4, found 870.3.

### IWR-PEG4-Pomalidomide (19b)

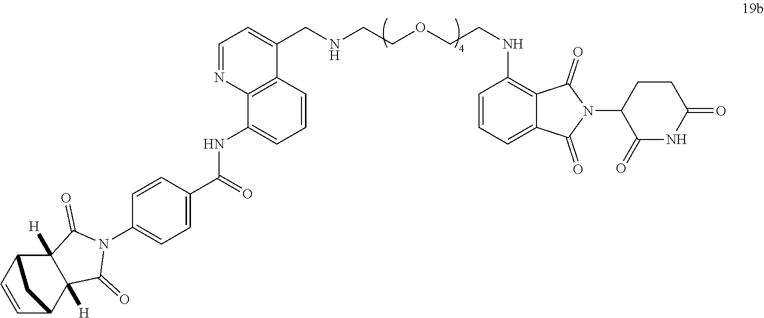

Prepared by essentially the same method as that for 19a. MS (ESI)⁺ calculated for C₄₉H₅₂N₇O₁₁ (M+H)⁺914.4, found 914.4.

### IWR-PEG1-Pomalidomide (19c)

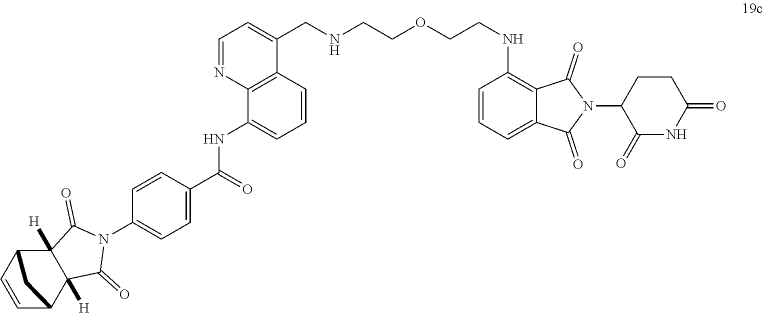

Prepared by essentially the same method as that for 19a. MS (ESI)⁺ calculated for C₄₃H₄₀N₇O₈ (M+H)⁺782.3, found 782.3.

### IWR-PEG2-Pomalidomide (19d)

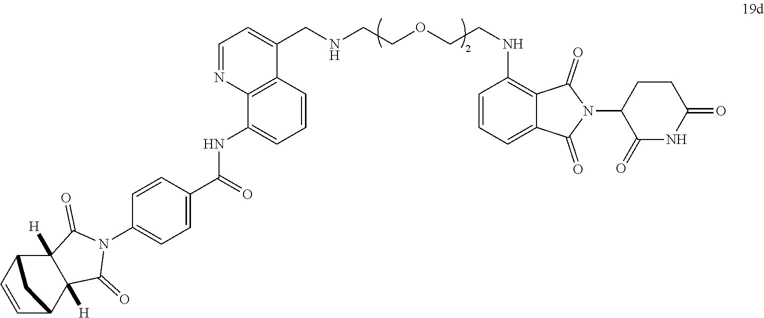

Prepared by essentially the same method as that for 19a. MS (ESI)⁺ calculated for C₄₅H₄₄N₇O₉ (M+H)⁺826.3, found 826.3.

### IWR-PEG5-Pomalidomide (19e)

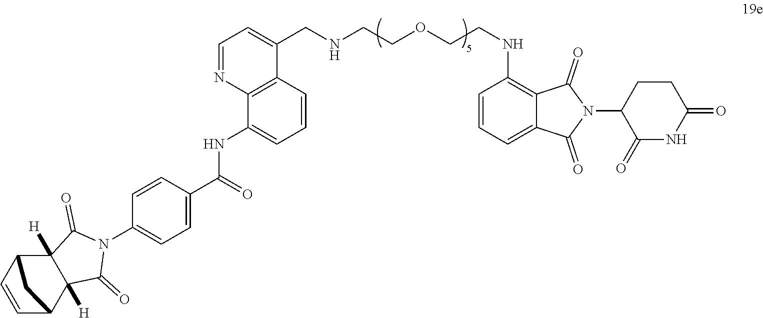

Prepared by essentially the same method as that for 19a. MS (ESI)⁺ calculated for C₅₁H₅₆N₇O₁₂ (M+H)⁺958.4, found 958.4.

### IWR-PEG2-Pomalidomide (19f)

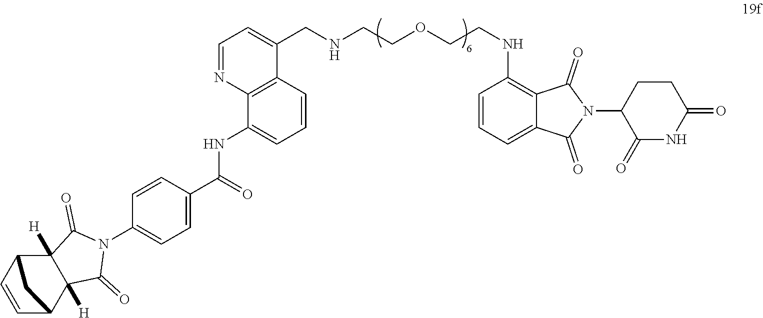

Prepared by essentially the same method as that for 19a. MS (ESI)⁺ calculated for C₅₃H₆₀N₇O₁₃ (M+H)⁺1002.4, found 1002.4.

### Example 5

Degradation of PARP5a/b using IWR-triazole-PEG4-pamolidomide. 3×10⁵ HEK293 or DLD-1 cells (1 mL) were seeded into each well of a 12-well plate and incubated for 24 h followed by treating with IWR-1, IWR-triazole-PEG4-pamolidomide (17c), IWR-PEG4-VHL-ligand (18c) or IWR-PEG4-pamolidomide (19b) at different concentrations for 24 h. The cells were then collected, lysed, and analyzed by SDS-PAGE.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments, exemplary embodiments and optional features, modification and variation of the concepts herein disclosed may be resorted to by those skilled in the art, and that such modifications and variations are considered to be within the scope of this disclosure as defined by the appended claims. The specific embodiments provided herein are examples of useful embodiments of the present disclosure and it will be apparent to one skilled in the art that the present disclosure may be carried out using a large number of variations of the devices, device components, methods steps set forth in the present description. As will be recognized by one of skill in the art, methods and devices useful for the present methods can include a large number of optional composition and processing elements and steps.

### VIII. REFERENCES

The following references to the extent that they provide exemplary procedural or other details supplementary to those set forth herein, are specifically incorporated herein by reference.
An et al., Nat Commun 8, 15398 (2017).
Berger et al., Br. J. Pharmacol. 175, 192-222 (2018).
Bondeson et al., Nat Chem Biol 11, 611-617 (2015).
Brochier et al., Proc. Natl. Acad. Sci. U.S.A. 112, 15220-15225 (2015).
Bryant et al., Nature 434, 913-917 (2005).
Burslem, G. M. & Crews, C. M. Chem. Rev. 117, 11269-11301 (2017).
Campeau et al., Hum. Genet. 124, 31-42 (2008).
Chambon et al., Biochem. Biophys. Res. Commun. 11, 39-43 (1963).
Chen et al., Nat. Chem. Biol. 5, 100-107, 2009.
Cyrus et al., Mol Biosyst 7, 359-364 (2011).
D'Amours et al., Biochem. J 342, 249-268 (1999).
Daniels et al., J Proteome Res 13, 3510-3522 (2014).
Dawson et al., Ann. Rev. Pharmacol. Toxicol., 57:437-454, 2017.
Dawson, T. M. & Dawson, V. L., Annu. Rev. Pharmacol. Toxicol. 57, 437-454 (2017).
Eliasson et al., Nat. Med. 3, 1089-1095 (1997).
Farmer et al., Nature 434, 917-921 (2005).
Ferraris, D. V., J. Med. Chem. 53, 4561-4584 (2010).
Gadd et al., Nat Chem Biol 13, 514-521 (2017).
Galdeano et al., J. Med. Chem. 57, 8657-8663 (2014).
Gibson, B. A. & Kraus, W. L., Nat Rev Mol Cell Biol 13, 411-424 (2012).
Gunaydin et al., PLoS ONE 7: e33740, 2012.
Haikarainen et al., Curr. Pharm. Des. 20, 6472-6488, 2014.
Hoch et al., Nature 541, 87-91 (2017).
Hottiger, M. O. Annu. Rev. Biochem. 84, 227-263 (2015).
Hu et al., J Proteome Res 15, 477-486 (2016).
Huang et al., Nature 461, 614-620, 2009.
Huang, X. & Dixit, V. M., Cell Res. 26, 484-498 (2016).
James et al., ACS Chem Biol 11, 3179-3190 (2016).
Kam et al., Science 362, eaat8407 (2018).
Kam et al., Science, 362(6414), 2018.
Karlberg et al., J. Med. Chem. 53: 5352-5355, 2010.
Kim, M. K. Oncol. Lett. 16, 6895-6902 (2018).
Kirby et al., Acta Cryst. F68: 115-118, 2012.
Knezevic et al., Cell Chem Biol 23, 1490-1503 (2016).
Krishnakumar, R. & Kraus, W. L. Mol. Cell 39, 8-24 (2010).
Kulak et al., Mol. Cell Biol. 35, 2425-2435, 2015.
Kulak et al., Mol. Cell Biol. 35: 2425-2435, 2015.
Kulak et al., Mol. Cell Biol. 35: 2425-2435, 2015.
Lai et al., Angew. Chem. Int. Ed. Engl. 55, 807-810 (2016).
Leidecker et al., Nat Chem Biol 12, 998-1000 (2016).
Leutert et al., Cell Rep 24, 1916-1929 e1915 (2018).
Li et al., Regul. Pept. 186, 83-91 (2013).
Liu et al., Nucleic Acids Res 45, 8129-8141 (2017).
Lord, C. J. & Ashworth, A. Science 355, 1152-1158 (2017).
Lu et al., Chem. Biol. 22, 755-763 (2015).
Lum and Chen, Curr. Med. Chem. 22, 4091-4103, 2015.
Lupo and Trusolino, Biochim. Biophys. Acta 1846, 201-215, 2014.
Lüscher et al., Chem. Rev., DOI: 10.1021/acs.chemrev.1027b00122, 2018.
Mariotti et al., Br. J. Pharmacol. 174, 4611-4636, 2017.
Martello et al., Nat Commun 7, 12917 (2016).
Miyamoto et al., Mol. Cell. Biol. 19, 2644-2649 (1999).
Murai et al., Cancer Res. 72, 5588-5599 (2012).
Murai et al., Mol Cancer Ther 13, 433-443 (2014).
Narwal et al., J Med. Chem. 55: 1360-1367, 2012.
Olson et al., Nat Chem Biol 14, 163-170 (2018).
Pal et al., Cancer 104, 2807-2816 (2005).
Pedrioli et al. EMBO Rep 19 (2018).
Pirinen et al., Cell Metab 19, 1034-1041 (2014).
Qiu et al., Acta Cryst. D70: 2740-2753, 2014.
Riffell et al., Nat. Rev. Drug Discov. 11, 923-936, 2012.
Rycker and Price, Mol. Cell Biol. 24, 9802-9812, 2004.
Schwanhausser, B. et al. Global quantification of mammalian gene expression control. Nature 473, 337-342 (2011).
Shieh et al., J Biol. Chem. 273, 30069-30072 (1998).
Thorsell et al., J. Med. Chem. 60, 1262-1271 (2017).
Thorsell et al., J. Med. Chem. 60: 1262-1271, 2017.
U.S. Pat. No. 5,635,517
U.S. Pat. No. 6,495,541
U.S. Patent Publication 20160272639
U.S. Patent Publication 20180228907
Wahlberg et al., Nat. Biotechnol. 30, 283-288 (2012).
Wahlberg et al., Nat. Biotechnol. 30, 283-288 (2012).
Wang et al., Exp. Neurol. 218, 193-202 (2009).
Wang et al., Science 354 (2016).
Winter et al., Science 348, 1376-1381 (2015).
Yamanaka et al., J Biol. Chem. 263, 3879-3883 (1988).
Yang et al., Shock 13, 60-66 (2000).
Yu et al., Science 297, 259-263 (2002).
Zhang et al., J. Clin. Invest. 121, 2447-2456 (2011).
Zhang et al., Nat Methods 10, 981-984 (2013).
Zhen et al., Cell Rep 21, 2326-2337 (2017).
Zhen, Y. & Yu, Y., Biochemistry (Mosc). 57, 429-440 (2018).
Zheng et al., Am J Physiol Renal Physiol 288, F387-398 (2005).

1. A compound of Formula (I), or a pharmaceutically acceptable salt thereof,
A-L-B (I)
wherein:
A is or comprises a binding moiety that binds to a PARP protein;
B is or comprises a binding moiety that binds to a E3 ligase for ubiquitin or a ubiquitin-like protein; and
L is a linker moiety that connects A and B in a manner that allows A and B to establish appropriate interactions with a PARP protein and an E3 ligase, respectively.

2. The composition of claim 1, wherein the PARP protein is PARP1.

3. The composition of claim 1, wherein the PARP protein is PARP2.

4. The composition of claim 1, wherein the PARP protein is PARP3.

5. The composition of claim 1, wherein the PARP protein is PARP5A.

6. The composition of claim 1, wherein the PARP protein is PARP5B.

7. The composition of claim 1, wherein the E3 ligase is protein cereblon (CRBN).

8. The composition of claim 1, wherein the E3 ligase is von Hippel Lindau disease tumor suppressor (pVHL).

9. The composition of claim 1, wherein the E3 ligase is mouse double minute 2 homolog (MDM2).

10. The composition of claim 1, wherein the E3 ligase is cellular inhibitor of apoptosis protein-1 (cIAP1).

11. The composition of claim 1, wherein the E3 ligase is beta-transducin repeat containing protein (β-TrCP).

12. The composition of claim 1, wherein A makes one or more interactions with human PARP1 at one or more sites selected from the group consisting of Glu763, Asp766, His862, Gly863, Arg878, Ala880, Gly888, Tyr889, Tyr896, Ser904, and Glu988.

13. The composition of claim 1, wherein A makes one or more interactions with human PARP2 at one or more sites selected from the group consisting of Gly429, Arg444, Tyr462, Ser470, and Tyr473.

14. The composition of claim 1, wherein A makes one or more interactions with human PARP3 at one or more sites selected from the group consisting of Leu287, Asp291, His384, Gly385, Thr386, Arg400, Tyr414, and Ser422.

15. The composition of claim 1, wherein A makes one or more interactions with human PARP5A at one or more sites selected from the group consisting of Gly1185, Asp1198, His1201, Tyr1203, Tyr1213, and Ser1221.

16. The composition of claim 1, wherein A makes one or more interactions with human PARP5B at one or more sites selected from the group consisting of Gly1032, Asp1045, His1048, Tyr1050, Tyr4060, and Ser1068.

17. The composition of claim 1, wherein A comprises rucaparib, veliparib, niraparib, olaparib, talazoparib, AG-14361, GPI 15427, GPI 16539, GPI 21016, CEP-3499, CEP-8983, PJ34, PD128763, NU1025, NU1085, PA-10, OL-1, STO1168, STO1131, STO1542, ME0327, ME0328, ME0352, ME0354, ME0355, ME0359, ME0368, ME0398, ME0400, isoindolin-1-one, 4-benzylphthalazin-1(2H)-one, 2,7,8,9-tetrahydro-3H-pyrido[4,3,2-de]phthalazin-3-one, 5-oxo-6,11-dihydro-5H-indeno[1,2-c]isoquinoline-9-sulfonamide, pyrazolo[1,5-a]quinazolin-5(4H)-one, or 3-oxo-2,3-dihydrobenzofuran-7-carboxamide.

18. The composition of claim 1, wherein A comprises IWR-1, IWR-3, IWR-6, IWR-8, XAV939, GNF-1331, GNF-6231, AZ-6102, TNKSi49, AZ-1366, AZ-6102, CMP4, CMP4b, CMP24, CMP40, JW-55, JW-74, G007-LK, K-756, NVP-TNKS656, UPF-1854, E7449, K-756, or WIKI-4.

19. The composition of claim 1, wherein B comprises thalidomide, pomalidomide, or lenalidomide.

20. The composition of claim 1, wherein B comprises a 4-hydroxyprolyl derivative.

21. The composition of claim 1, wherein L comprises a linear chain with a formula of —[(CH₂)ₘ₁—X₁]ₙ₁—[(CH₂)ₘ₂—X₂]ₙ₂—[(CH₂)ₘ₃—X₃]ₙ₃—[(CH₂)ₘ₄ X₄]ₙ₄, wherein [(CH₂)ₘ₁—X1]ₙ₁ is covalently bound to A, [(CH₂)ₘ₄—X₄]ₙ₄ is covalently bound to B, each m1, m2, m3, and m4 is independently 0, 1, 2, 3, 4, 5, 5, 7, 8, 9, or 10, each n1, n2, n3, and n4 is independently 0, 1, 2, 3, 4, 5, 5, 7, 8, 9, or 10, and each X1, X2, X3, and X4 is independently absent (a bond), O, S, NH, NR, C(O), C(O)O, OC(O), C(O)NH, NHC(O), C(O)NR, N(R)C(O), 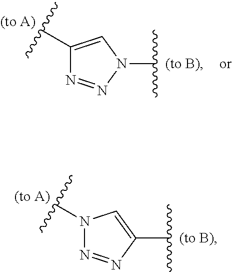
wherein R is C₁₋₆alkyl, C₁₋₆alkyl selectively functionalized with one or more halogen, thiol, hydroxyl, carbonyl, carboxyl, carbonyloxyl, C₁₋₆alkoxy, C₁₋₆hydroxyalkoxy, amino, C₁₋₆alkylamino, di(C₁₋₆alkyl)amino, or azido groups, C₃₋₅alkenyl, C₃₋₅alkynyl, oligo(ethylene glycol), or poly(ethylene glycol).

22. The composition of claim 1, wherein L is or comprises an oligo(ethylene glycol) chain.

23. The composition of claim 21, wherein R is optionally conjugated to an antibody.

24. The compound of claim 1, wherein the compound of formula (IIa), (IIb), (IIc), (IId), (IIe), (IIf), (IIg), (IIh), (IIi), or (IIj), or a pharmaceutically acceptable salt thereof:

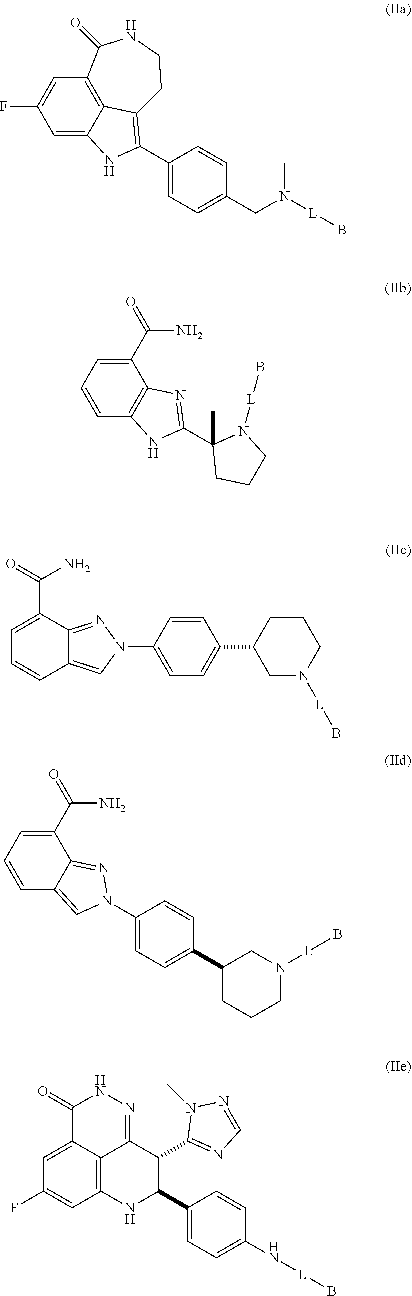

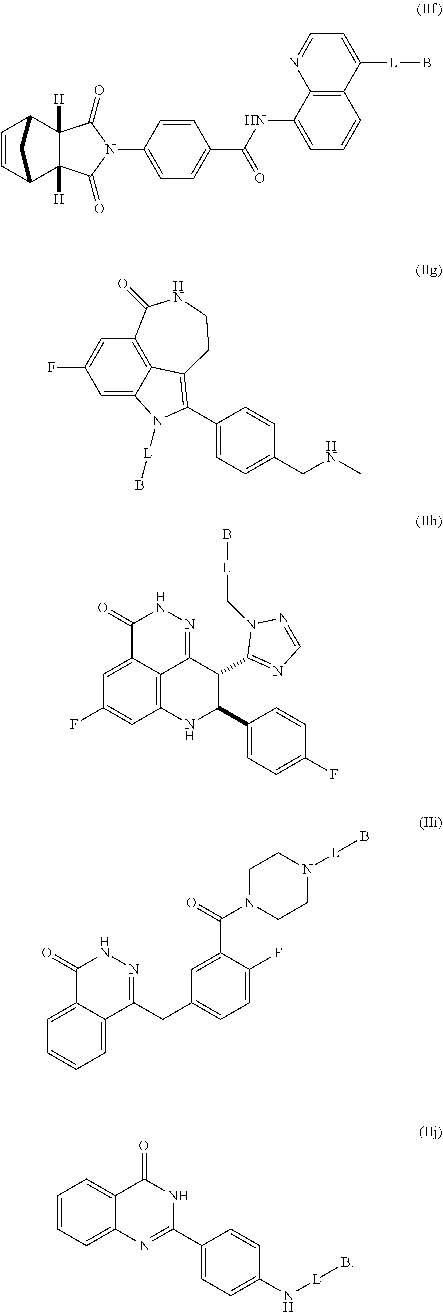

25. The compound of claim 1, wherein the compound of formula (IIIa), (IIIb), (IIIc), (IIId), (IIIe), (IIIf), (IIIg), (IIIh), (IIIi), or (IIIj), or a pharmaceutically acceptable salt thereof:

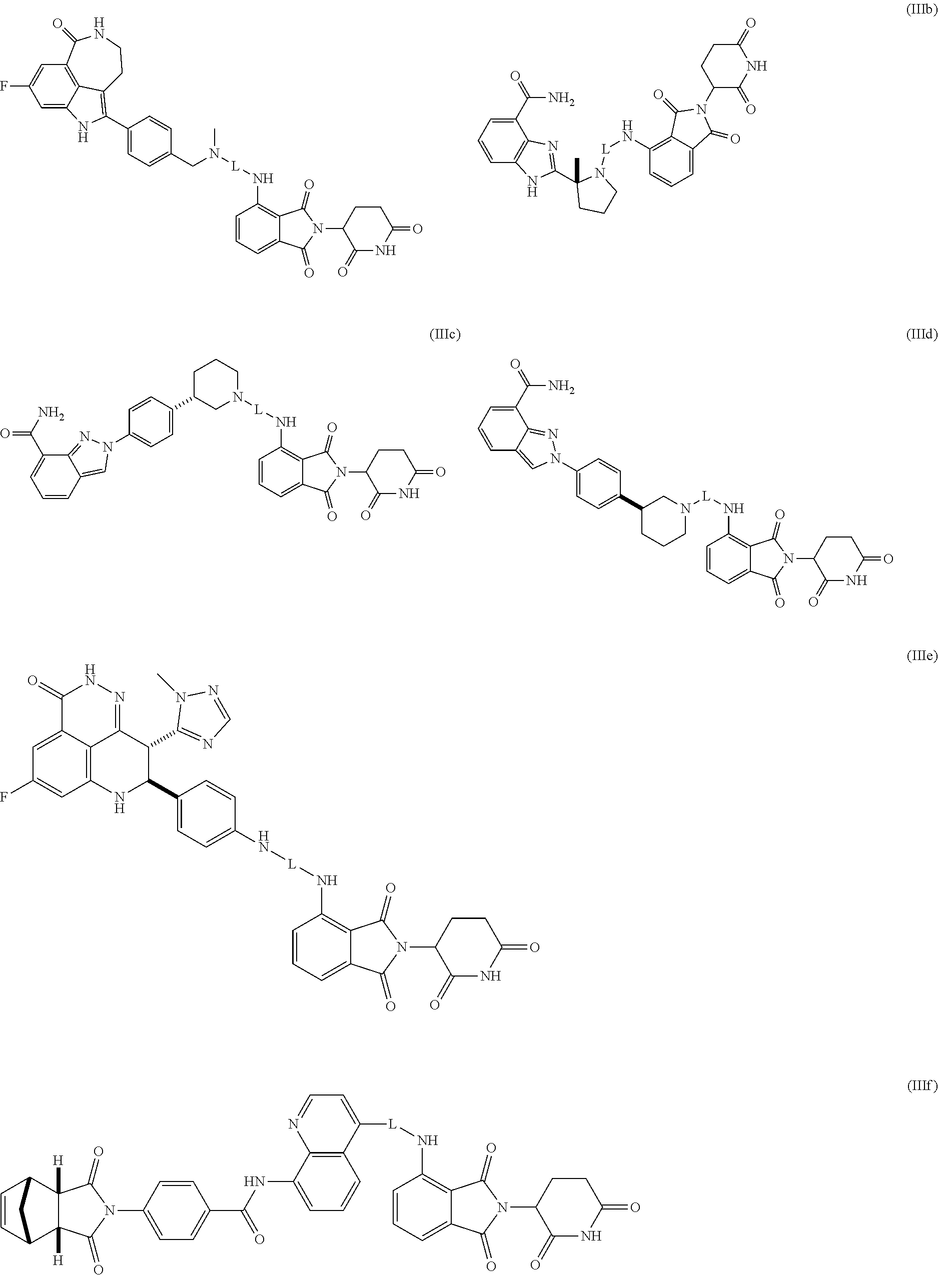

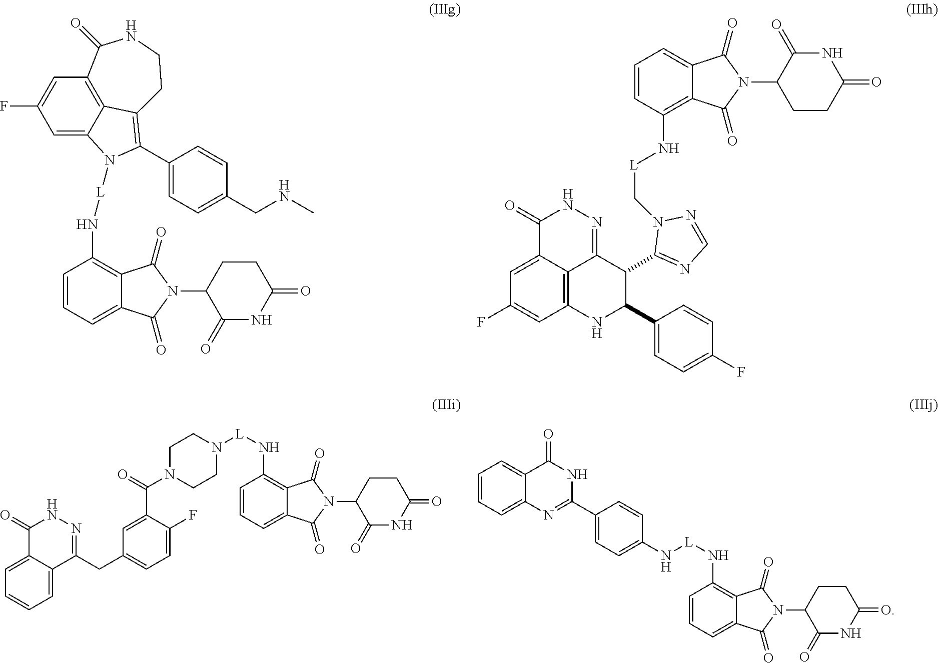

26. The compound of claim 1, wherein the compound of formula (IVa), (IVb), (IVc), (IVd), (IVe), (IVf), (IVg), (IVh), (IVi), or (IVj), or a pharmaceutically acceptable salt thereof:

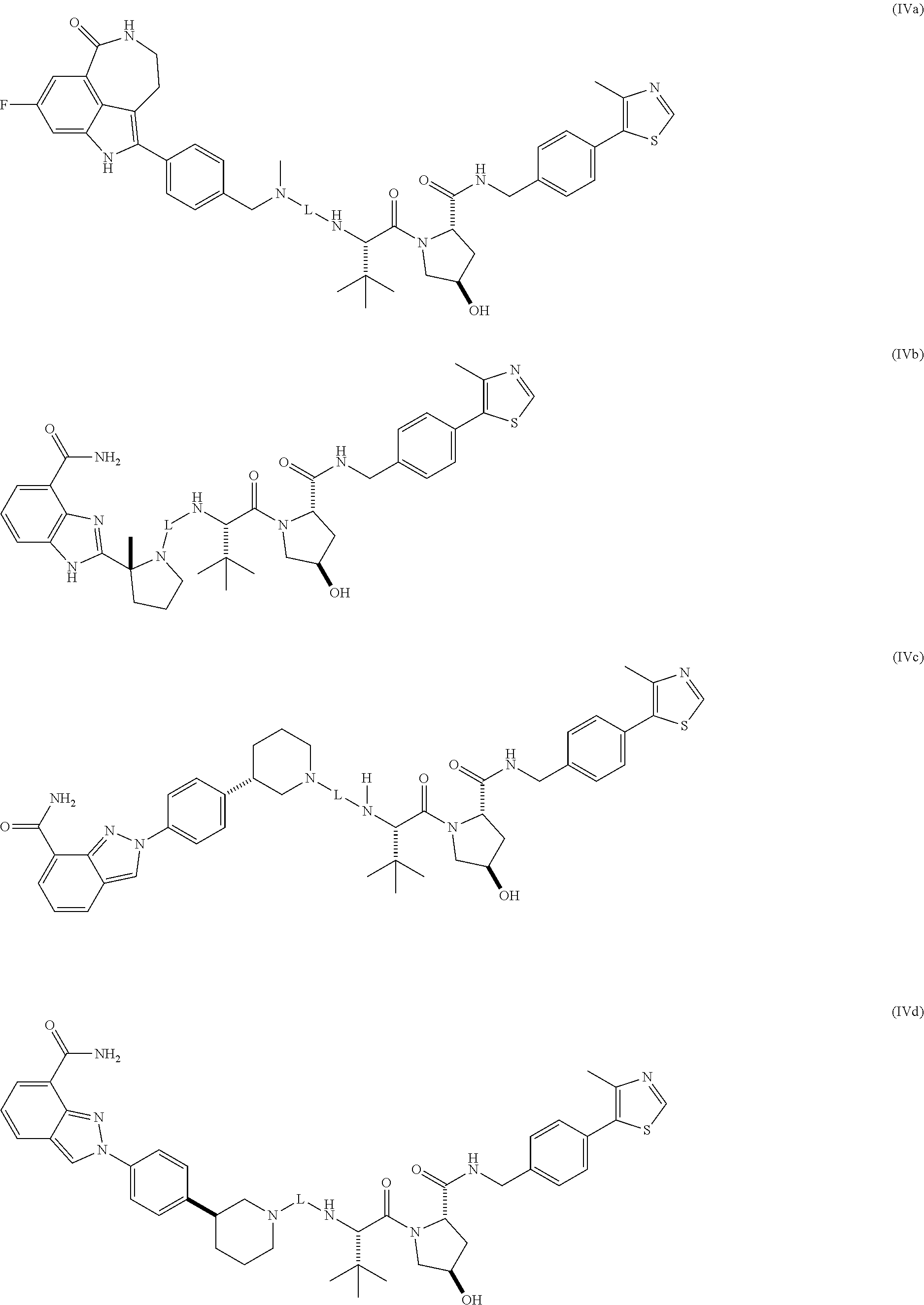

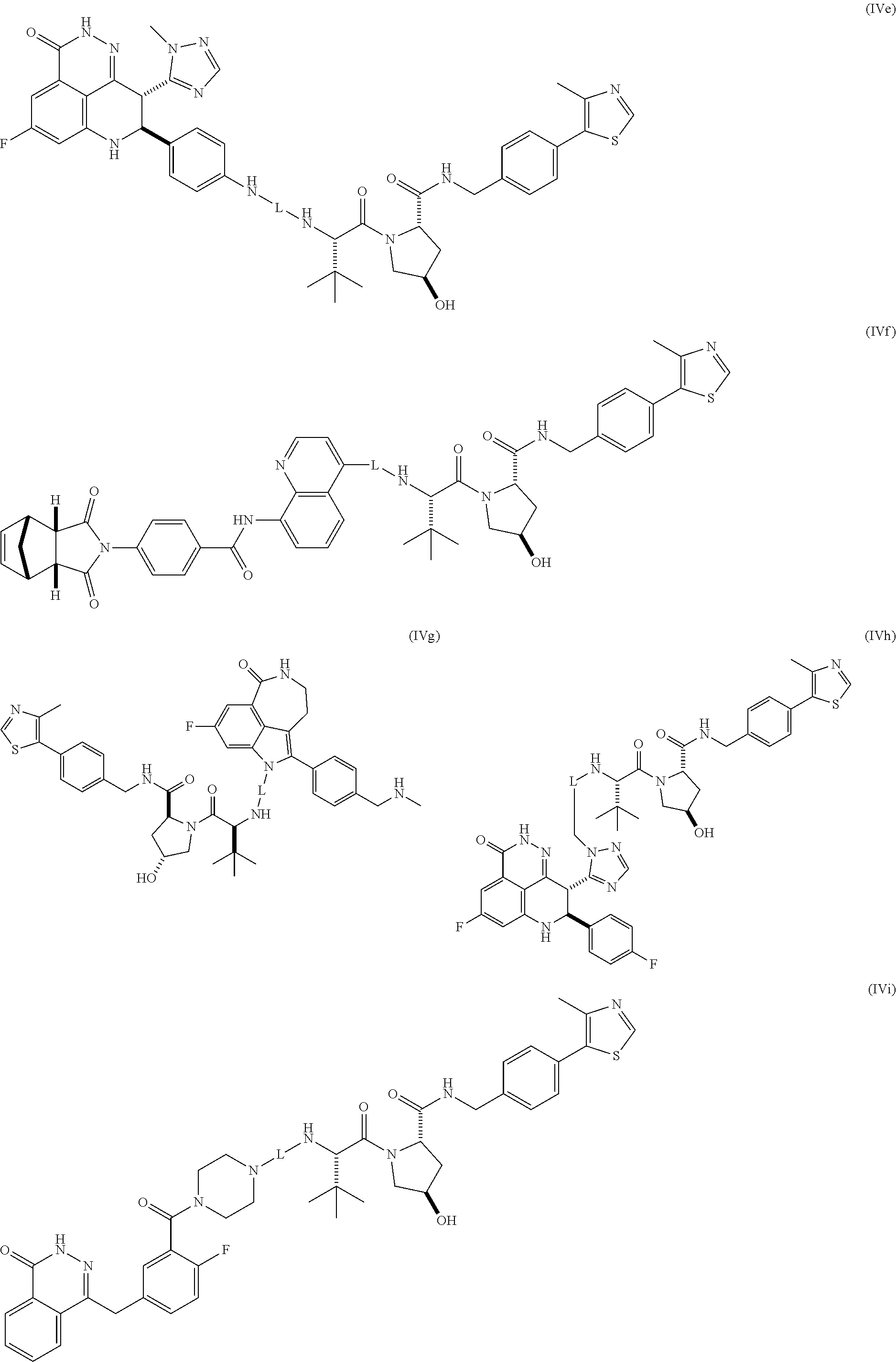

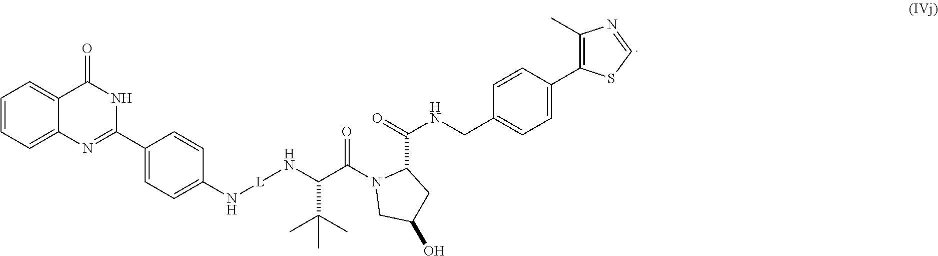

27. The compound of claim 1, wherein the compound of formula (Va), (Vb), (Vc), (Vd), (Ve), (Vf), (Vg), (Vh), (Vi), (Vj), (Vk), (Vl), (Vm), or (Vn), or a pharmaceutically acceptable salt thereof, wherein n is 0, 1, 2, 3, 4, 5, 5, 7, 8, 9, or 10:

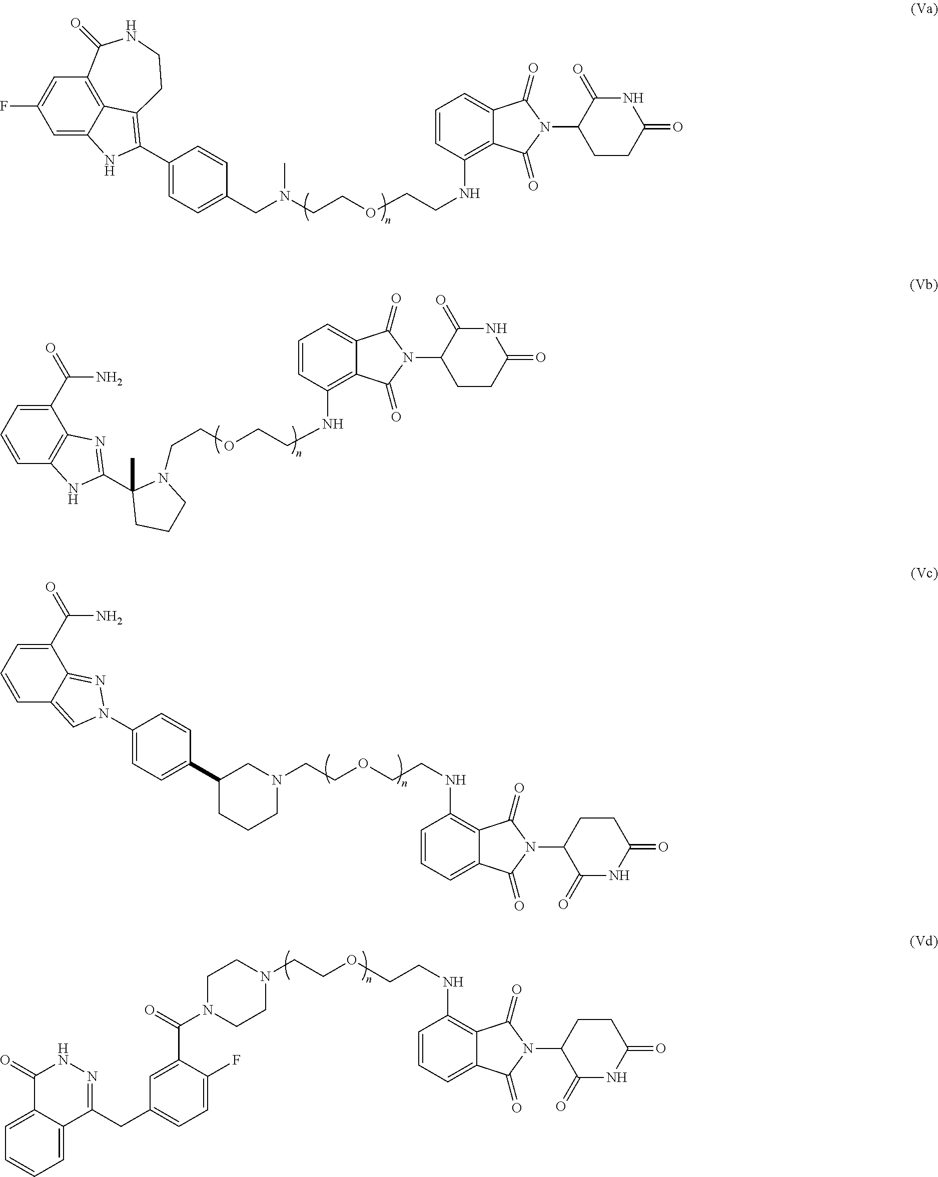

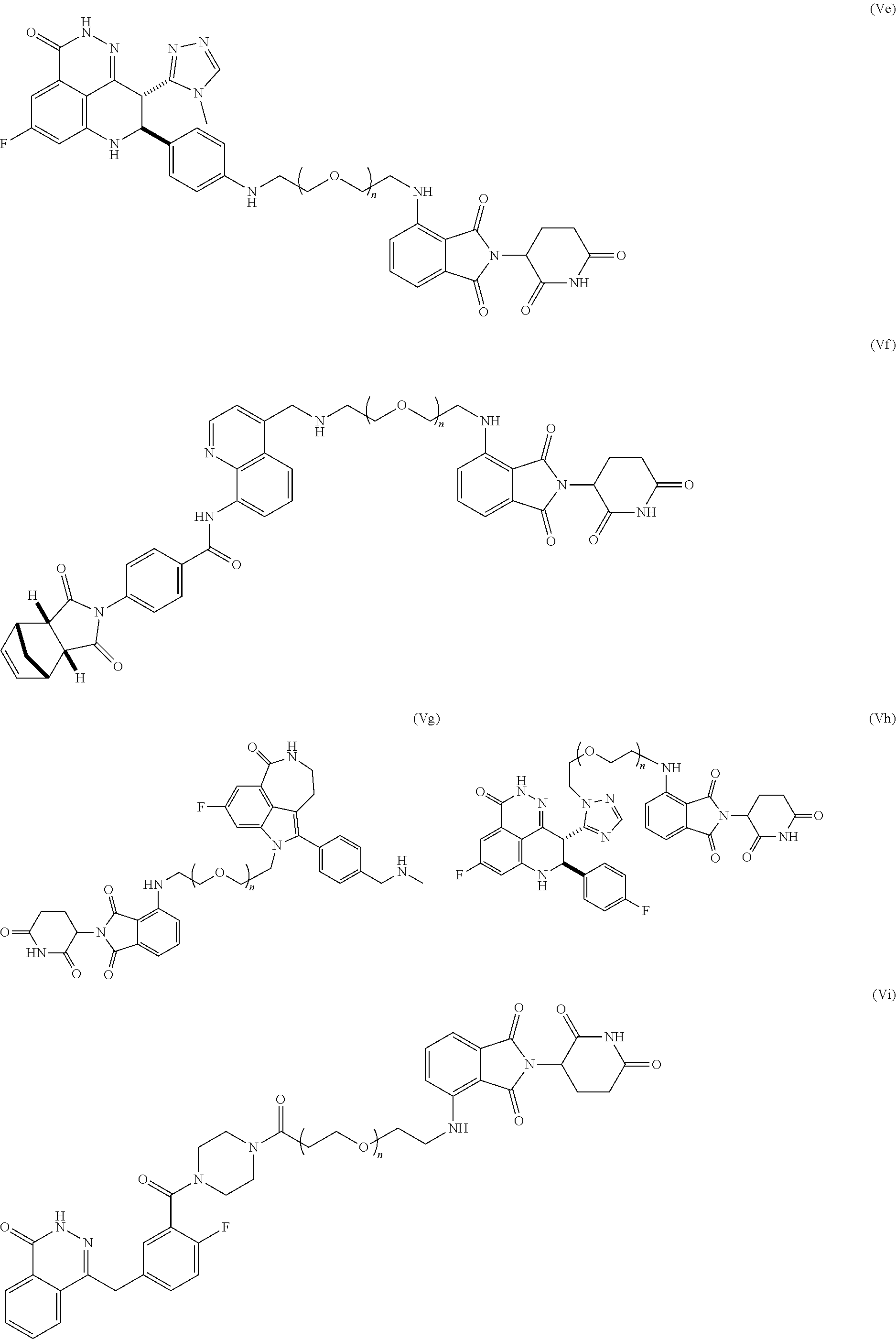

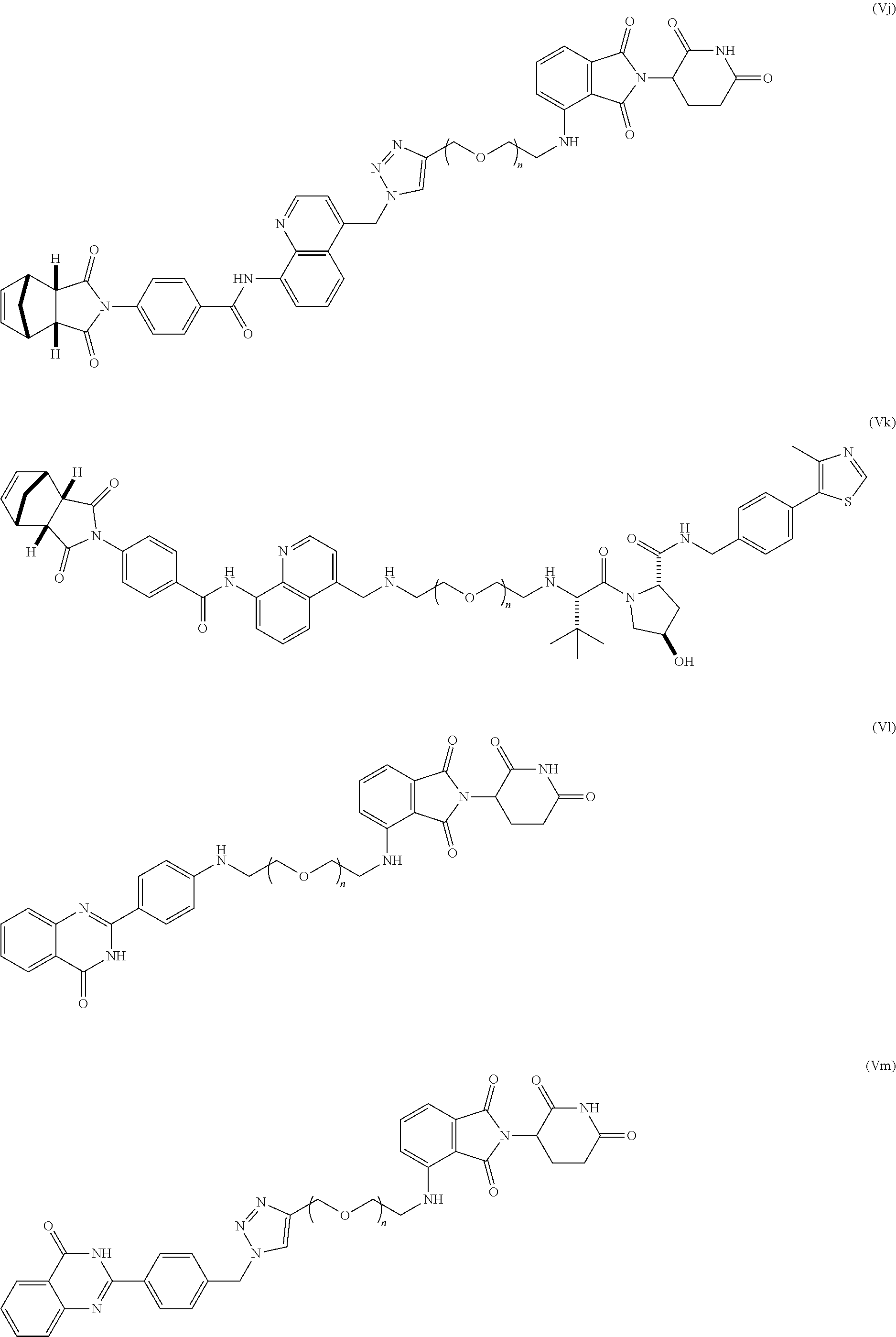

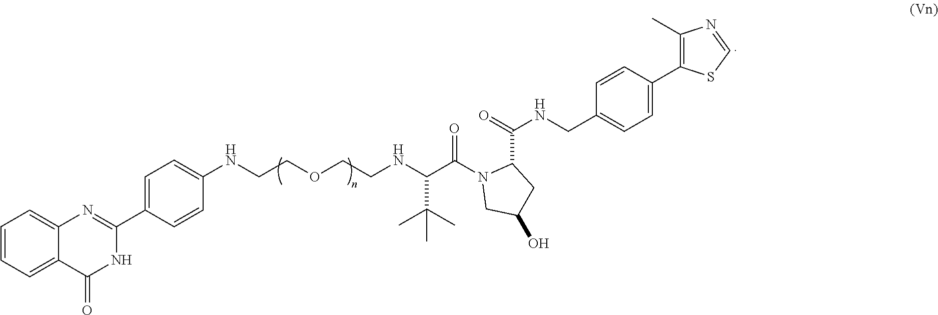

28. A compound of claim 1 selected from the group consisting of:

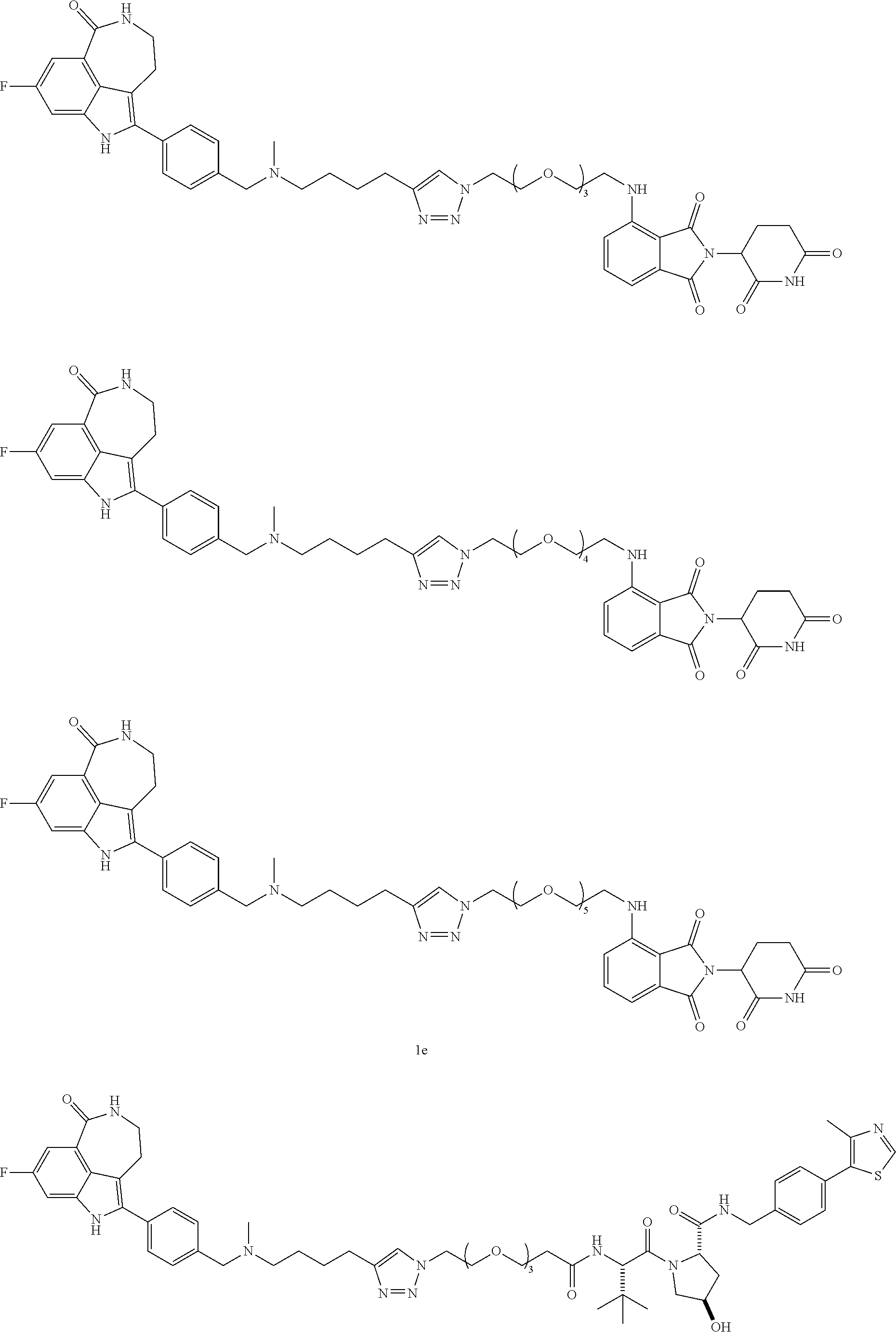

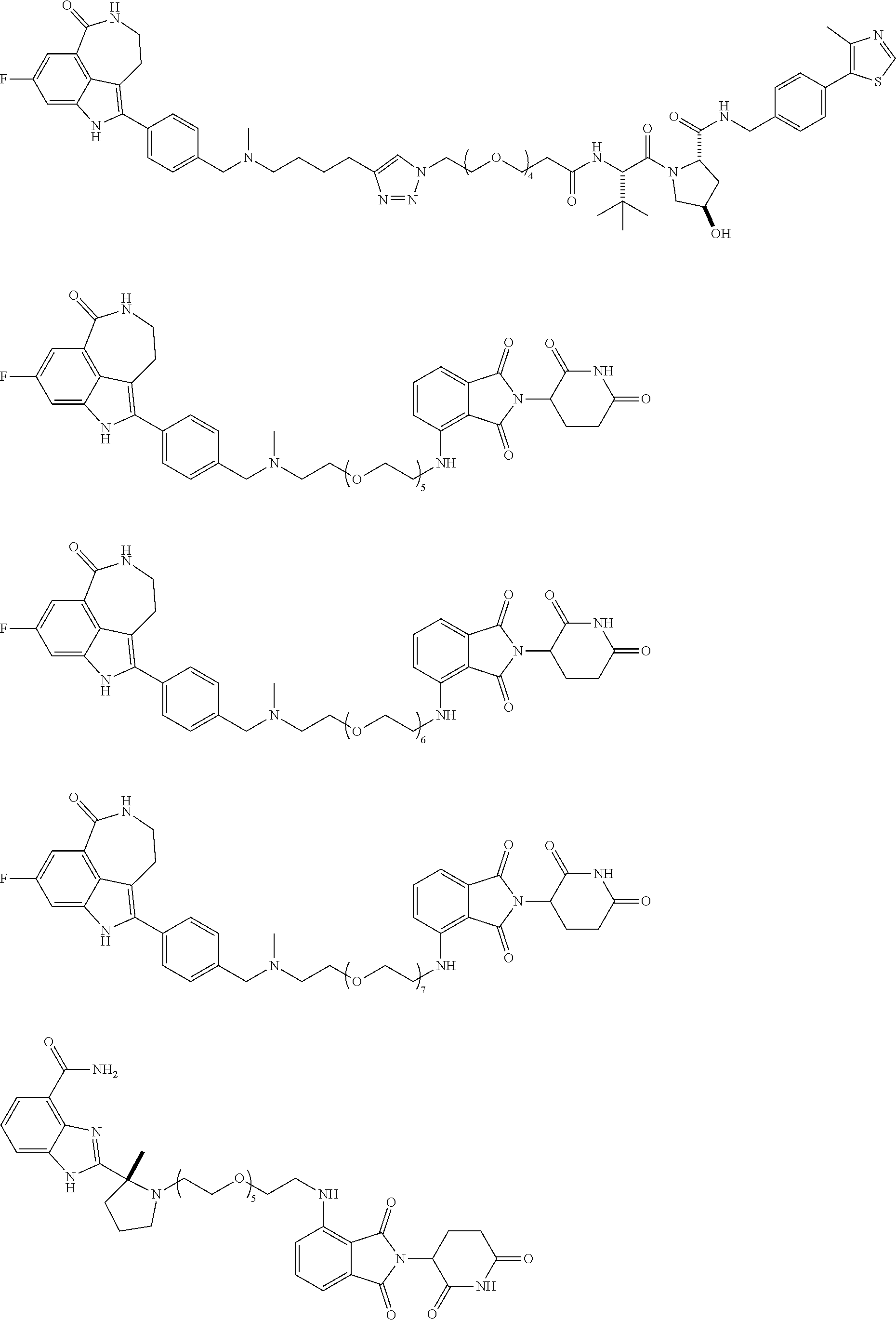

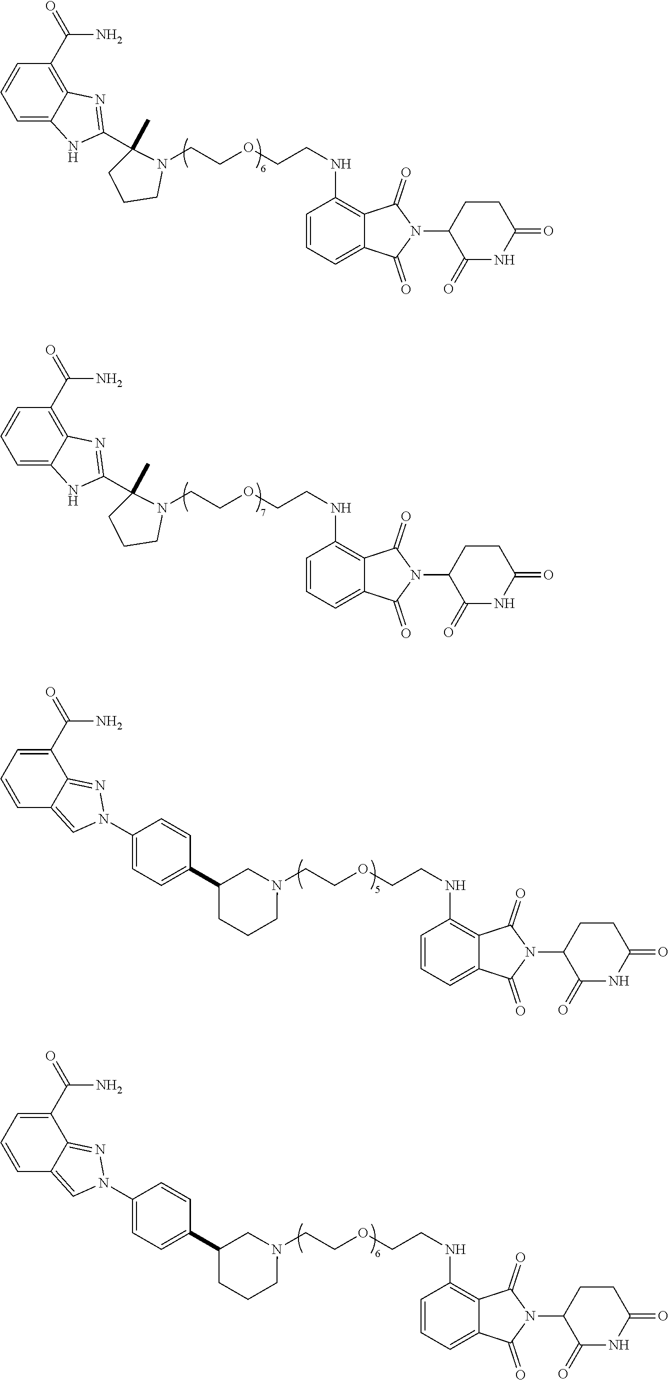

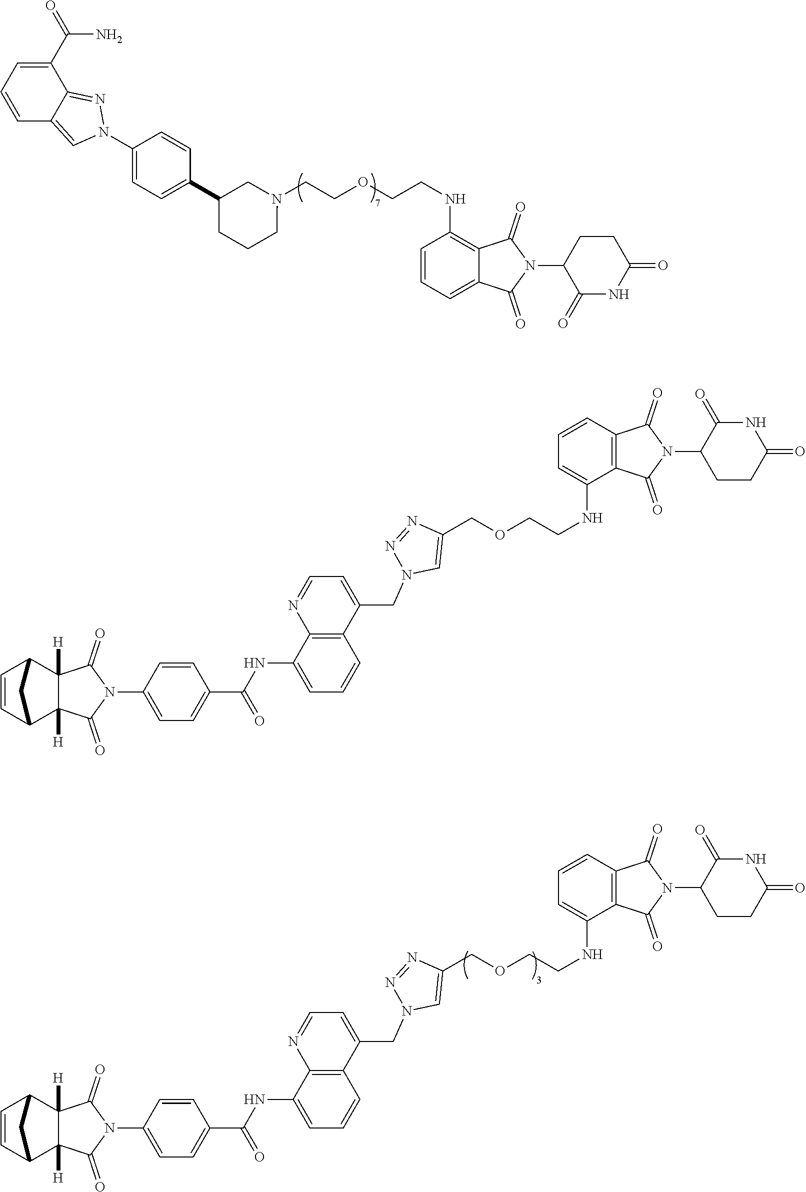

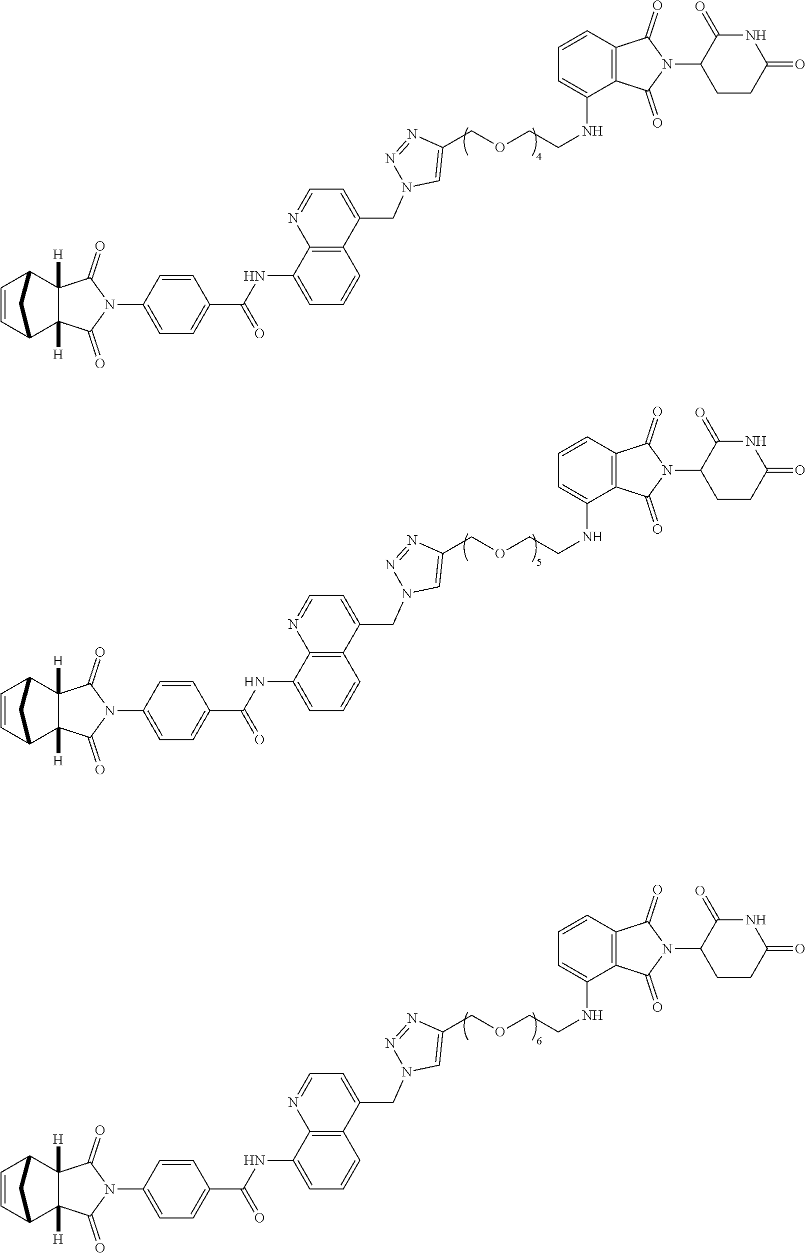

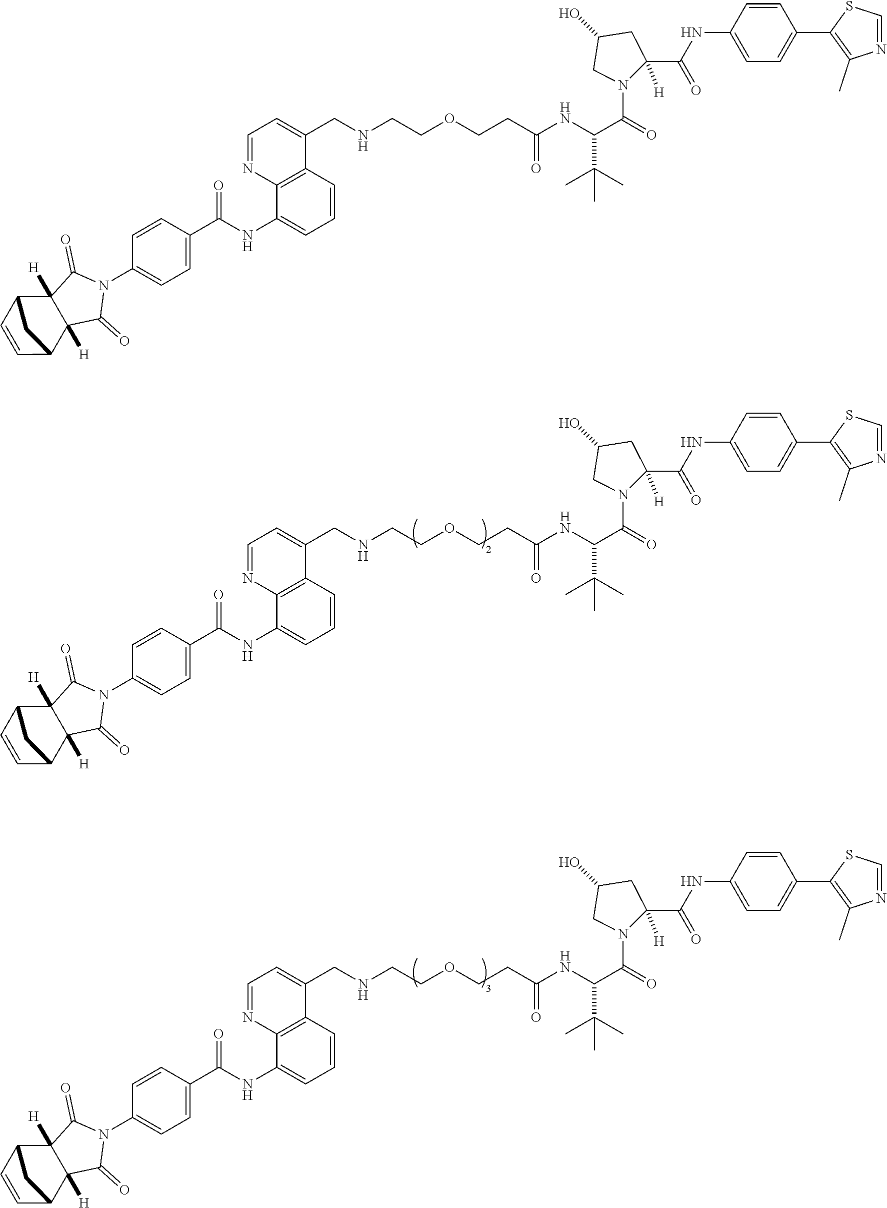

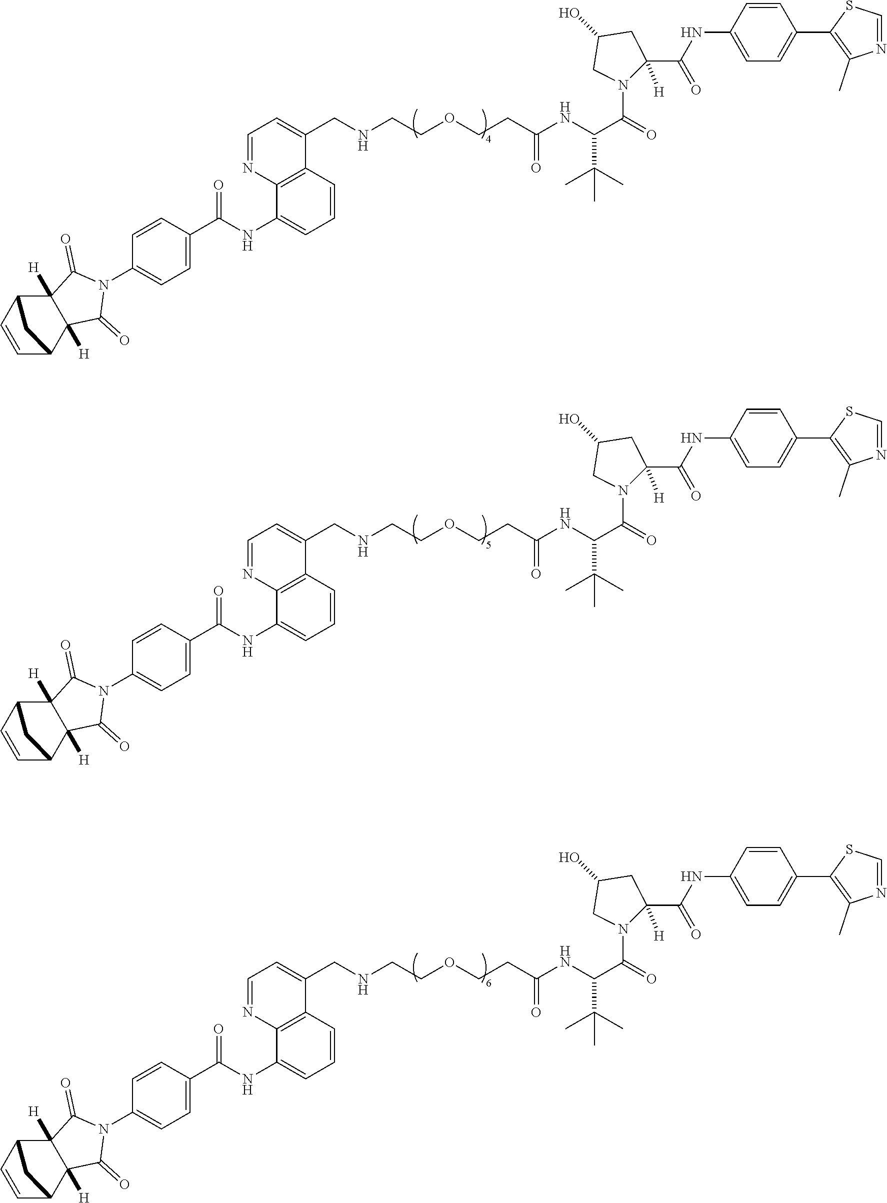

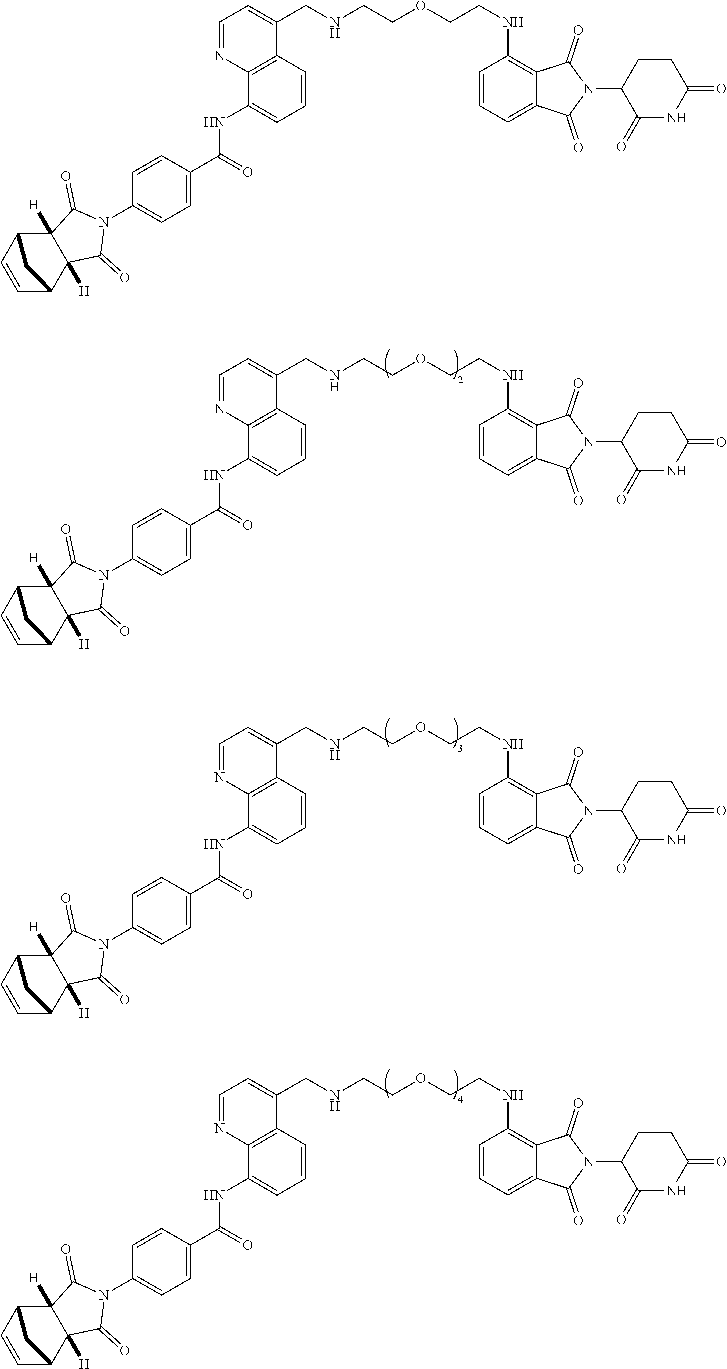

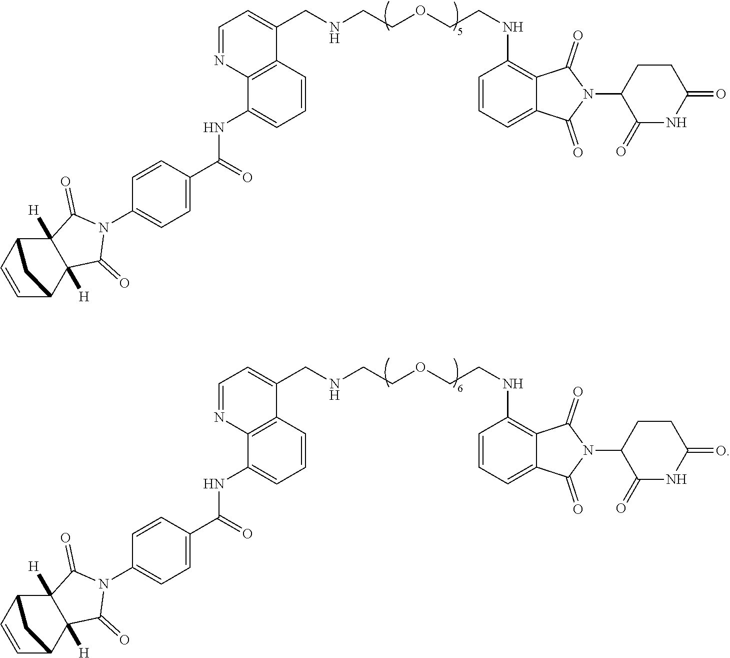

29. A compound of claim 1 selected from the group consisting of:

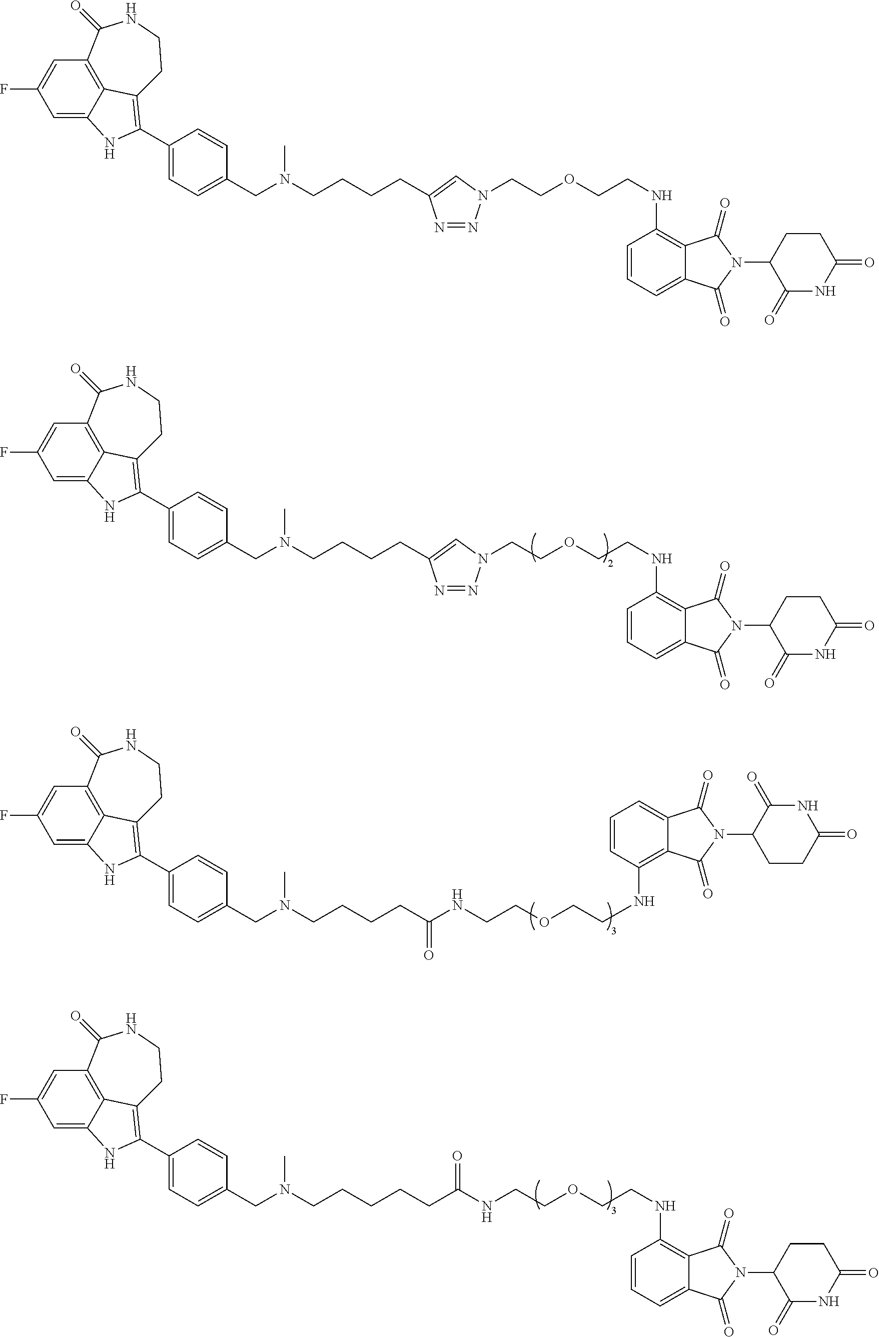

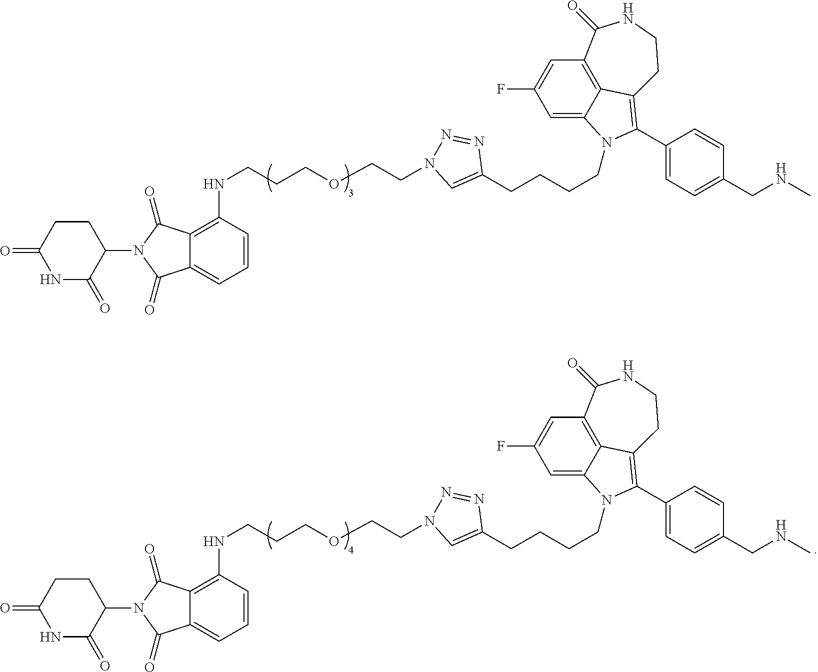

30. A compound of claim 1 selected from the group consisting of:

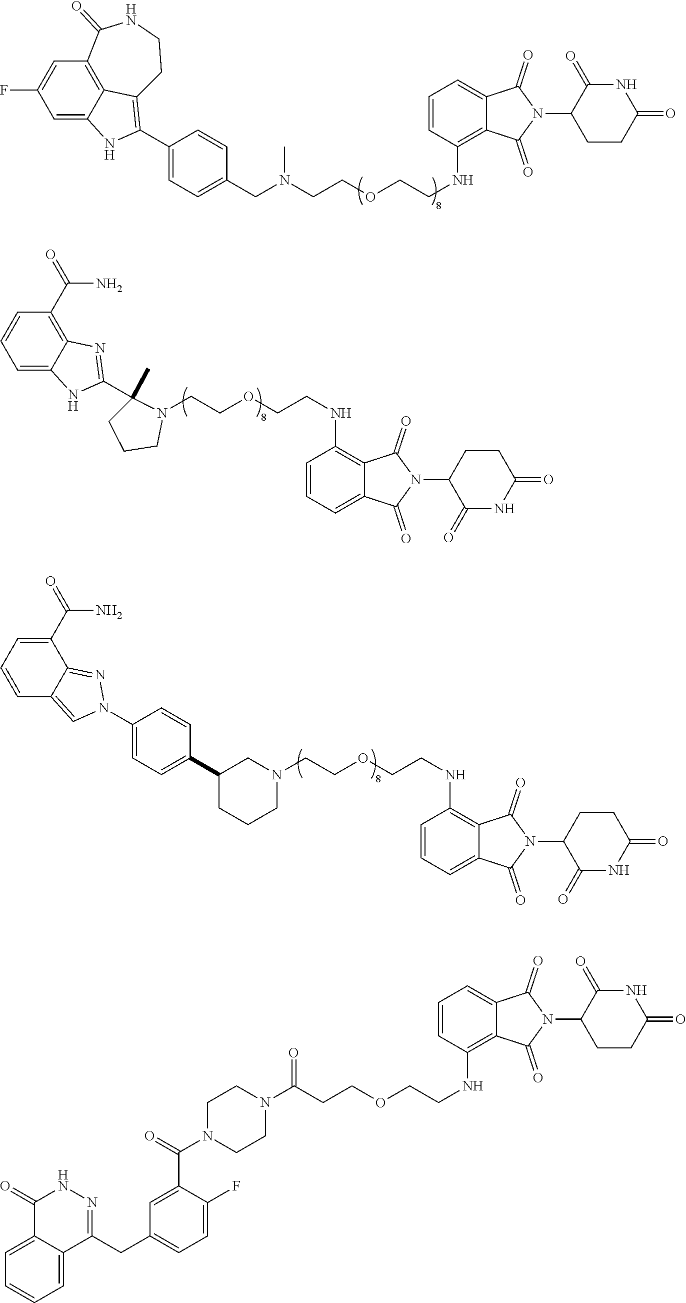

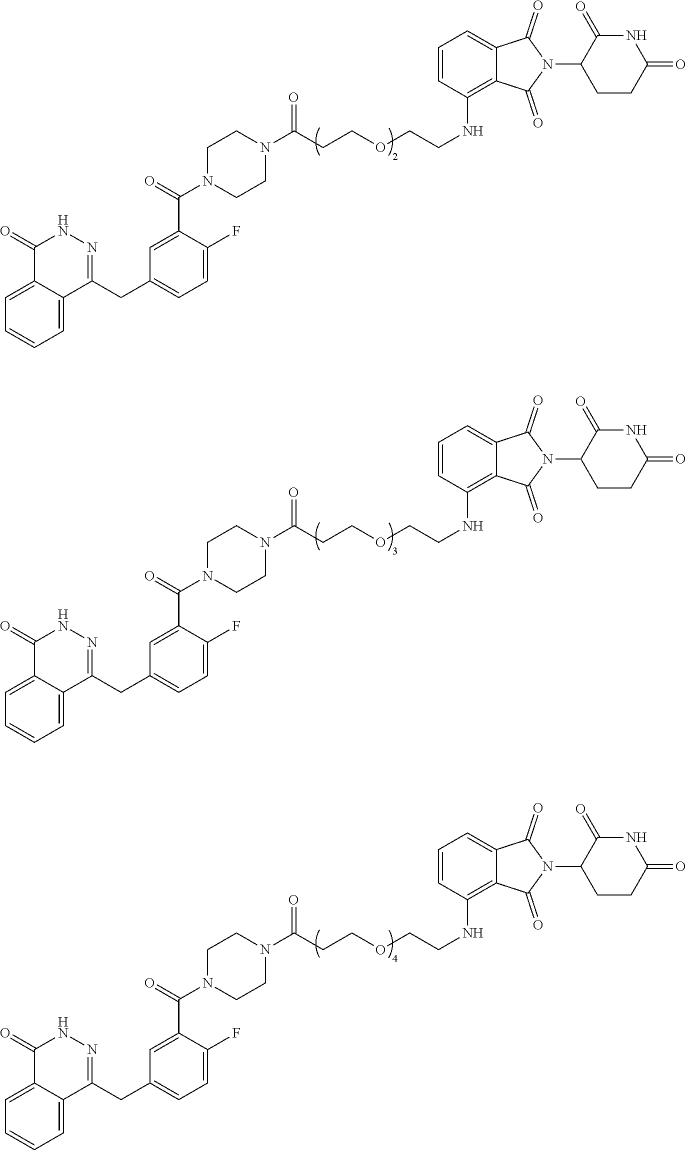

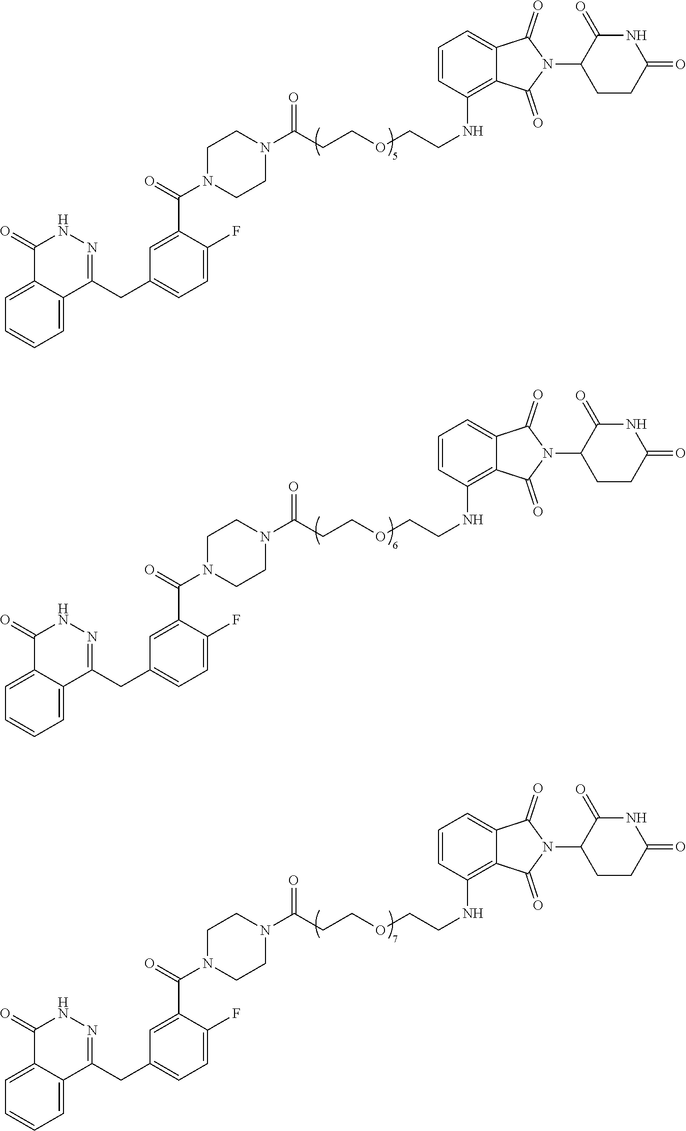

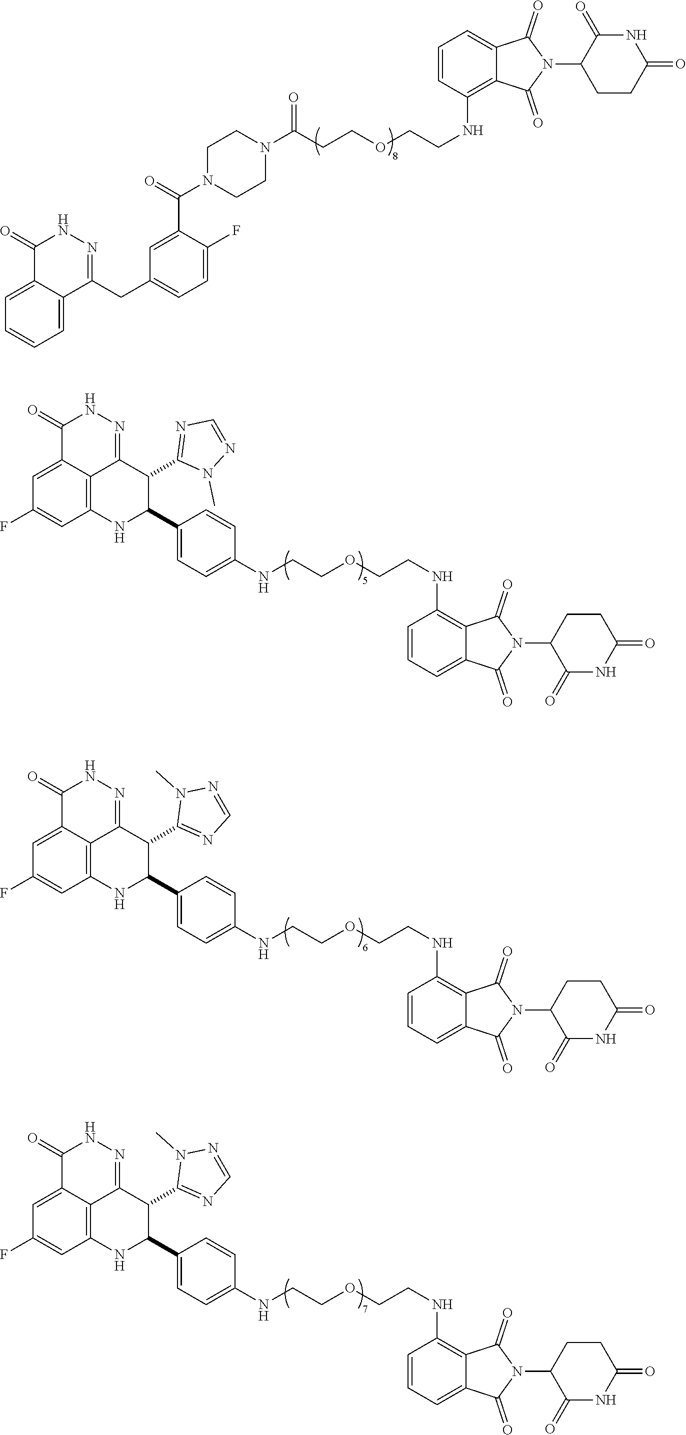

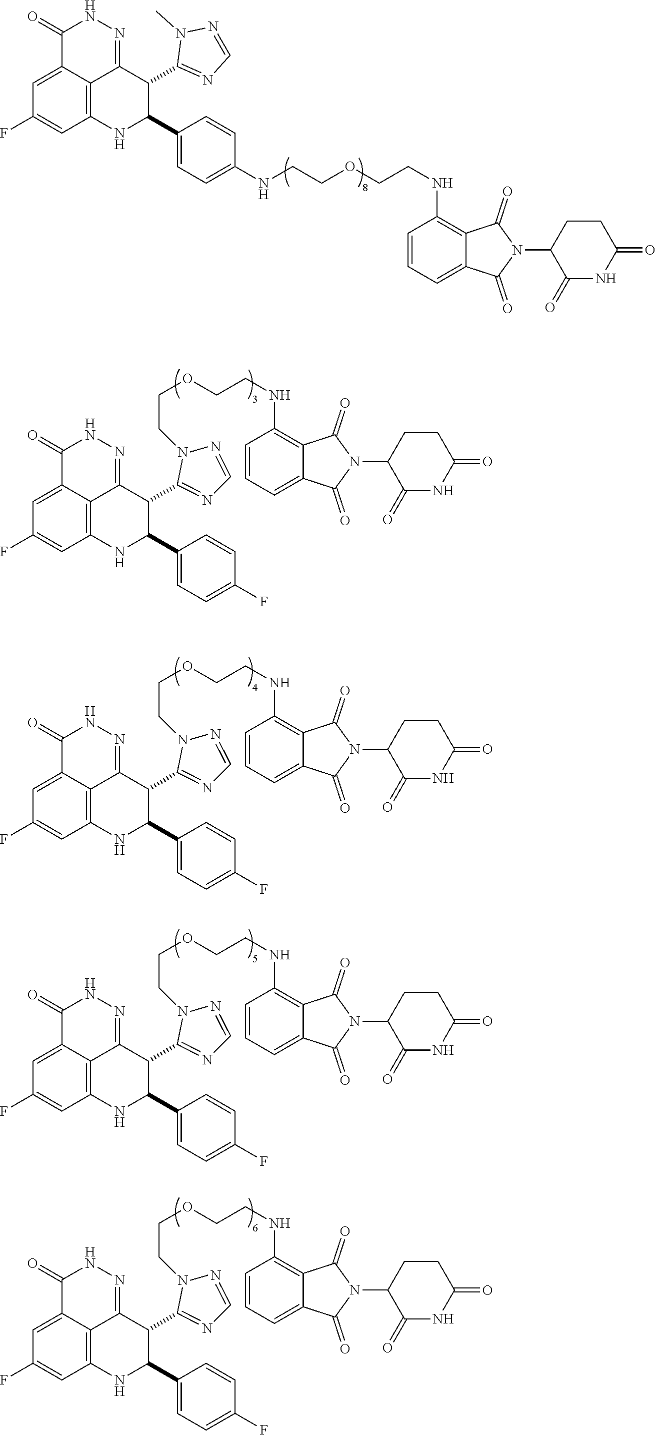

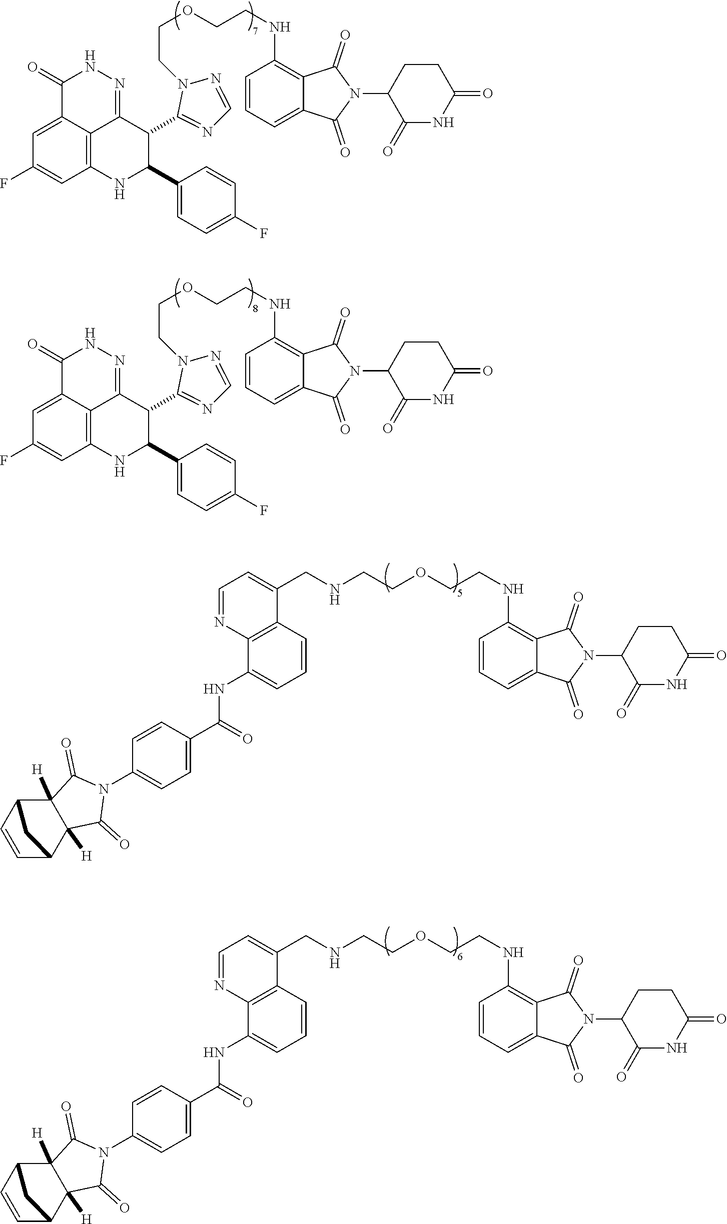

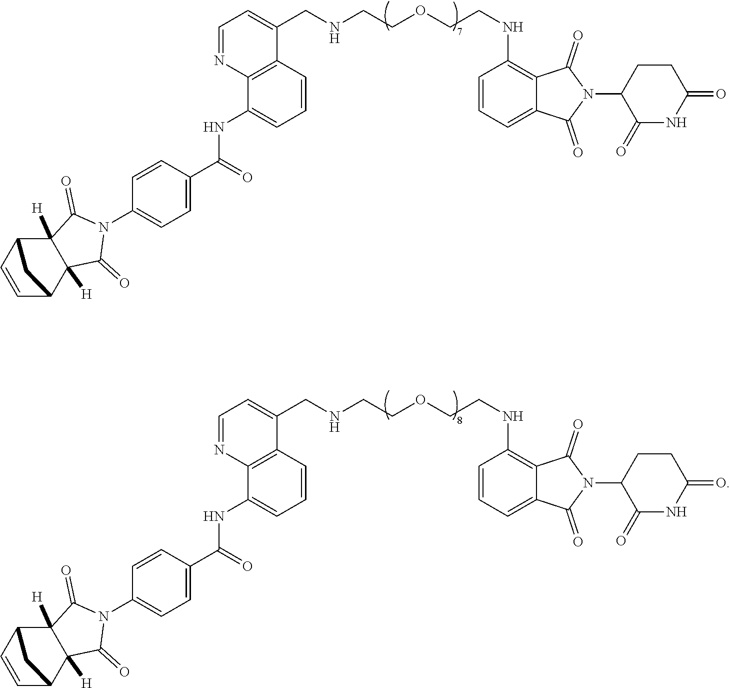

31. A pharmaceutical composition comprising a compound according to claim 1 and one or more pharmaceutically acceptable excipients.

32. A pharmaceutical composition comprising a compound according to claim 1 and at least one further therapeutic agent and one or more pharmaceutically acceptable excipients.

33. The method of inducing covalent modification of one or more surface-accessible lysine residues of a PARP protein comprising administering to a patient in need thereof, an effective amount of a compound according to claim 1, or a pharmaceutically acceptable salt thereof.

34. The method of claim 33, wherein the covalent modification is attachment of one or more ubiquitin molecules.

35. The method of claim 33, wherein the covalent modification is attachment of one or more SUMO molecules.

36. The method of claim 33, wherein the covalent modification is attachment of one or more ISG15 molecules.

37. The method of claim 33, wherein the covalent modification is attachment of one or more NEDD8 molecules.

38. The method of claim 33, wherein the covalent modification is attachment of one or more ATG8 molecules.

39. The method of claim 33, wherein the covalent modification is attachment of one or more ATG12 molecules.

40. The method of claim 33, wherein the covalent modification is attachment of one or more FAT10 molecules.

41. The method of claim 33, wherein the covalent modification is attachment of one or more HUB1 molecules.

42. The method of claim 33, wherein the covalent modification is attachment of one or more MNSFB molecules.

43. The method of claim 33, wherein the covalent modification is attachment of one or more UFM1 molecules.

44. The method of claim 33, wherein the covalent modification is attachment of one or more URM1 molecules.

45. A method of reducing the amount of one or more PARP proteins comprising administering to a patient in need thereof, an effective amount of a compound according to claim 1, or a pharmaceutically acceptable salt thereof.

46. A method of modulating protein PARylation comprising administering to a patient in need thereof, an effective amount of a compound according to claim 1, or a pharmaceutically acceptable salt thereof.

47. A method of treating a heart disease comprising administering to a patient in need thereof an effective amount of a compound according to claim 1, or a pharmaceutically acceptable salt thereof.

48. A method of treating ischemia-reperfusion injury comprising administering to a patient in need thereof an effective amount of a compound according to claim 1, or a pharmaceutically acceptable salt thereof.

49. The method of claim 48, wherein the ischemia-reperfusion injury is due myocardial infarction, stroke, heart surgery, or trauma.

50. The method of claim 48, wherein the ischemia-reperfusion injury is the brain, heart, muscle, lung, kidney, liver, pancreas, or intestine.

51. A method of treating cancer comprising administering to a patient in need thereof an effective amount of a compound according to claim 1, or a pharmaceutically acceptable salt thereof.

52. The method of claim 51, wherein the cancer is a solid cancer, such as lung cancer, prostate cancer, pancreatic cancer, liver cancer, brain cancer, ovarian cancer, uterine cancer, testicular cancer, breast cancer, endometrial cancer, skin cancer, head and neck cancer, stomach cancer, or colon cancer.

53. The method of claim 51, further comprising administering a second cancer therapy to said patient, such as a chemotherapy, a radiotherapy, an immunotherapy, a toxin therapy or surgery.

54. A method of treating fibrosis comprising administering to a patient in need thereof an effective amount of a compound according to claim 1, or a pharmaceutically acceptable salt thereof.

55. A method of treating metabolic syndrome comprising administering to a patient in need thereof an effective amount of a compound according to claim 1, or a pharmaceutically acceptable salt thereof.

56. A method of treating diabetes comprising administering to a patient in need thereof an effective amount of a compound according to claim 1, or a pharmaceutically acceptable salt thereof.

57. A method of treating a developmental disorder comprising administering to a patient in need thereof an effective amount of a compound according to claim 1, or a pharmaceutically acceptable salt thereof.

58. A method of treating an inflammatory disease comprising administering to a patient in need thereof an effective amount of a compound according to claim 1, or a pharmaceutically acceptable salt thereof.

59. A method of treating a neurological disorder comprising administering to a patient in need thereof an effective amount of a compound according to claim 1, or a pharmaceutically acceptable salt thereof.

60. A method of treating septic shock comprising administering to a patient in need thereof an effective amount of a compound according to claim 1, or a pharmaceutically acceptable salt thereof.

61. A method of treating acute pancreatitis comprising administering to a patient in need thereof an effective amount of a compound according to claim 1, or a pharmaceutically acceptable salt thereof.

62. A method of treating acute lung injury comprising administering to a patient in need thereof an effective amount of a compound according to claim 1, or a pharmaceutically acceptable salt thereof.

63. A method of treating non-alcoholic fatty liver disease (NASH) comprising administering to a patient in need thereof an effective amount of a compound according to claim 1, or a pharmaceutically acceptable salt thereof.

64. The method of claim 33, wherein compound is administered more than once, such as continuously over at least an hour or on a chronic basis.
